(19) United States
(12) Patent Application Publication
(10) Pub. No.: US 2023/0293504 A1
(43) Pub. Date: Sep. 21, 2023

(54) TARGETING SUCNR1 TO REDUCE NEUROINFLAMMATION

(71) Applicant: New York University, New York, NY (US)

(72) Inventors: Xin Li, New York, NY (US); Deepak Saxena, New York, NY (US); Yuqi Guo, New York, NY (US)

(21) Appl. No.: 18/186,698

(22) Filed: Mar. 20, 2023

Related U.S. Application Data
(60) Provisional application No. 63/321,752, filed on Mar. 20, 2022.

(51) Int. Cl. A61K 31/4375; A61P 29/00; A61K 47/34; A61P 25/00

(52) U.S. Cl. CPC A61K 31/4375; A61P 29/00; A61K 47/34; A61P 25/00

(57) ABSTRACT

The present disclose provides methods for treating neuroinflammation. Compositions having therapeutic agents targeted to interfering with succinate/succinate receptor signaling are administered to reduce or ameliorate neuroinflammation. The neuroinflammation may be associated with, caused by, or result from diseases/pathologies/disorders associated with the central nervous system.

### CROSS REFERENCE TO RELATED APPLICATIONS

This application claims priority to U.S. Provisional Application No. 63/321,752, filed on Mar. 20, 2022, the disclosure of which is incorporated by reference.

### BACKGROUND OF THE DISCLOSURE

Accumulated clinical and experimental studies support that chronic neuroinflammation is an important factor contributing to the development and progression of neurodegenerative diseases that are not hereditary. It has also been elucidated that neuroinflammation is associated with peripheral inflammation. The immune response within the Central Nervous System (CNS) involves local elements and elements that are transported from the periphery. The local CNS immune system is primarily composed of glial cells; microglia are the resident macrophages of the CNS. Immune disorders in the CNS often have their cause and origin outside the brain. Immune cells and cytokines are transported from the periphery, across the blood-brain barrier (BBB) and into the brain. This transport is strictly regulated but regulatory defects can lead to disease, and activation of brain-resident T cells and B cells as a result of influx from the periphery (or of primary brain processes) can lead to many neurodegenerative diseases. Oral-to-gut and gut-to-oral microbial transmissions can shape and/or reshape the microbial ecosystem in both habitats, eventually modulating local to systemic inflammation. Gastrointestinal tract (GI tract, digestive tract, alimentary canal) is the tract or passageway of the digestive system that leads from the mouth to the anus and importantly, GI pathogens can activate host immune responses. The GI microbiome and chronic inflammation during oral disease can induced cytokines which can also be disseminated via the bloodstream to trigger inflammatory responses in distant organs such as brain.

Succinate, an intermediate in the tricarboxylic acid (TCA) cycle is known to play an essential role in adenosine triphosphate (ATP) generation in mitochondria. As an important metabolite in both host and microbial metabolic processes, succinate could affect the host-bacteria interactions to promote pathogenesis. There are significant associations between succinate and certain aspects of the gut microbiome, such as the balance between succinate-consuming and succinate-producing bacteria. Moreover, succinate can penetrate BBB. Involvement of succinate signaling through SUCNR1 in various diseases have highlighted a pro-inflammatory role of succinate/SUCNR1 signaling. GI bacteria and their products such as succinate can affect systemic health through SUCNR1 signaling. Moreover, GI microbiome and chronic inflammation (such as during oral diseases or diabetes for example) can induce cytokines that can then be disseminated via the bloodstream to trigger inflammatory responses in brain. Therefore, the succinate elevation in GI tract and blood can eventually reach CNS and mediate the impact of peripheral inflammation on CNS.

### BRIEF SUMMARY OF THE DISCLOSURE

SUCNR1, and SUCNR1 antagonists in particular, can be an important therapeutic target for the treatment of many inflammatory human diseases.

The present disclosure provides methods and compositions/formulations comprising therapeutic agents targeted to interfering with succinate/succinate receptor signaling. These methods and compositions can be used for treatment or prevention of neurological disease and neuroinflammation. The methods and compositions can also be used for veterinary applications.

In an aspect, this disclosure provides a method for treating neurological disease and neuroinflammation by suppressing the activation of SUCNR1.

In one aspect, this disclosure provides a method of treatment of neuroinflammation. The method may comprise administering to an individual in need of treatment a composition comprising a succinate/succinate receptor 1 inhibitor. Any succinate/succinate receptor 1 inhibitor known in the art may be used. The composition is administered to a location other than the brain of the individual. The composition may be administered systemically or via topical application.

It is considered that the composition/formulations of the present disclosure can act to suppress succinate/SUCNR1 activation to alleviate and/or treat neurological disease and neuroinflammation. In an aspect, this disclosure provides a composition (e.g., an oral topical gel or composition suitable for systemic administration) comprising an inhibitor of the succinate/SUCNR1 pathway.

In various embodiments, the present disclosure provides formulations (e.g., formulations, such as, for example, compositions suitable for systemic administration or oral formulations, which may be gels) comprising therapeutic agents targeted to interfere with succinate/succinate receptor signaling. For example, the present disclosure provides compositions (e.g., systemic compositions or oral formulations, such as, for example, gels) comprising compounds having the following formula:

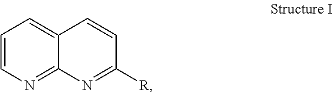

where R is chosen from a hydrogen atom, an alkyl group (e.g., methyl, ethyl, propyl, isopropyl, and the like), a COOR′ group, and

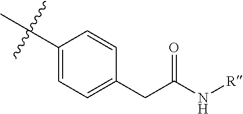

where R′ is an alkyl group (e.g., methyl, ethyl, propyl, isopropyl, and the like), R″ is selected from the group consisting of a hydrogen atom, an alkyl group (e.g., methyl, ethyl, propyl, isopropyl, and the like), and

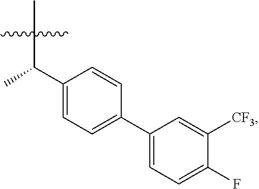 [SMILES: *[C@@H](C)c1ccc(-c2ccc(F)c(C(F)(F)F)c2)cc1]

The compound may be a salt, such as, for example, a succinate salt.

In an embodiment, the disclosure provides compositions (e.g., gels, such as, for example, oral gel formulations) comprising inhibitors of the succinate/SUCNR1 pathway. In various examples, the inhibitors may be compounds 7a (may be referred to herein as cpd 7a or cpd-7a) and/or 7b (may be referred to herein as cpd7a or cpd-7b).

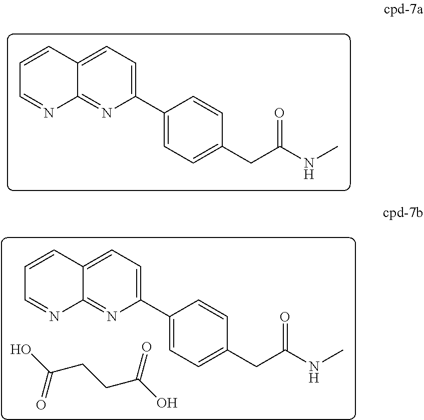

The compositions may comprise various polymers or gel precursors. Examples of such polymers include, but are not limited to poly lactic glycolic acid (PLGA) (e.g., having an M_{w} of 50,000 to 75,000 Da), polycaprolactone (PCL) (e.g., poly-ε-caprolactone ester terminated) (e.g., having an M_{w} of 14,000 Da), poly(D,L-lactide-co-glycolide) (which may be ester terminated), poly(D,L-lactide) (which may be ester terminated) (e.g., having an M_{w} of 50,000-75,000 Da), chitosan, starch, polylactic acid, alginate, and the like, and combinations thereof.

### DESCRIPTION OF THE DRAWINGS

For a fuller understanding of the nature and objects of the disclosure, reference should be made to the following detailed description taken in conjunction with the accompanying figures.

FIG. 1. Periodontitis induced neuroinflammation in mice. A. A schematic model shown control mice (healthy) versus mice with periodontitis (Perio). B. Representative μCT images and C. quantitative results of maxillae. (**** p<0.0001); D. Iba1 immunostaining of brain sections. IL1β protein levels in E. hippocampus; F. cortex; and G. cerebellum of mice. (** p<0.01, * p<0.05).

FIG. 2. Succinate elevation in periodontitis. Succinate levels A. in the sub-gingival plaques of patients. B. in the serum of mice. (****p<0.0001, **p<0.01).

FIG. 3. Inflammation and dysbiosis induced by succinate. Mice received daily intraperitoneal (IP) injection with PBS or Succinate (4 mM/kg) for 4 weeks before sample collection. A. Serum IL1β levels (**p<0.01). B. Alpha Diversity measured by the Observed number of ASVs, and Phylogenetic diversity index for oral swab samples. C. PCoA plots for Beta diversity based on the Weighted UniFrac distance matrix (p=0.04) and Bray-Curtis dissimilarity matrix (p=0.01). D. Network plot generated in Cytoscape 3.8.2. The small gray dots represent ASVs, the white circles represent WT samples, the yellow triangles represent WT_Suc samples. Edges represent the interaction of sample and ASV are colored based on sample type.

FIG. 4. Succinate elevation correlates with periodontitis pathogens in patients. Correlation-based network plot of the top 40 genera and succinate level in patient subgingival plaque samples.

FIG. 5. Fusobacterium nucleatum growth and virulence gene expression. A. F. nucleatum growth in media supplemented with indicated succinate concentrations shown as optical density measures over time (n=3, SEM). Different letters indicate statistically different from one another (ANOVA, p<0.05 by post-hoc Tukey's). B Relative expressions of virulence genes of F. nuleactum in 100 uM of succinate as determine by RT-PCR. (mean±SEM. ** p<0.01, * p<0.05).

FIG. 6. Enriched genera of periodontal pathogens in subjects with positive CSF β-amyloid. Mean-relative-abundance patterns for genera distinguish. Heatmap of the top 20 genera hierarchically clustered based on relative abundance normalized by row z-score in AbN and old AbP groups (N=21/group). The color patterns vary according to the groups indicating different bacterial compositions at genus level between the groups. Differential mean relative abundance at genus level between groups was assessed using Mann-Whitney U test.

FIG. 7. Significant difference between the succinate and gut microbiome of young and old mice. A. Succinate levels in young and old mice. B Boxplots of alpha diversity measured by the number of observed Amplicon Sequence Variants (ASVs), Shannon, and Faith's phylogenetic diversity (PD). Differences between groups were assessed by t test with Bonferroni corrected p-values reported. Significance is shown with asterisks: **** p<0.0001. C. PCoA plots for Beta diversity measured by Weighted Unifrac distance indicate significant differences in their microbial compositions between the young and old mice. Differences between clusters were calculated with permutational multivariate analysis of variance (Adonis, vegan package v2.5-7).

FIG. 8. Mean-relative-abundance patterns for genera distinguish young and old mice. A hierarchical-clustering relative-abundance heat map of the 20 (A) and 40 (B) most relatively abundant genera in the two cohorts with row z-score is displayed. Differential mean relative abundance at genus level between groups was assessed using Mann-Whitney U test.

FIG. 9. SUCNR1 expression in microglial cells. Single-molecule RNA in situ hybridization (RNA_ISH) was performed in accordance with RNAscope LS multiplex Fluorescent assay protocols USM #322800 (Advanced Cell Diagnostics, Hayward, CA, USA) on Leica Bond Rx Automated System. The sample slide was stained with RNAscope 2.5 LS Probe-Mm-Sucnr1-C3 (green), Fc receptor-like S, scavenger receptor (Mm-Fcrls-C4) and DAPI (Advanced Cell Diagnostics, Hayward, CA, USA) in microglia. (A) the cortex of a 12-week-old C57/B6 mouse brain; (B) Cultured primary microglia cells (C) Immunofluorescence staining of SUCNR1 protein (red) in Iba1+primary microglial cells derived from neonatal C57/B6 mouse pups and seeded into 8-well glass chamber slide.

FIG. 10. SUCNR1 regulates the stimulation of IL-1β in microglia cells. The levels of IL-1β were determined by ELISA (R&D Systems Cat #MLB00C). Primary cultured microglia cells were derived from postnatal wild type and SucnR1 knock out mouse brain. Microglia cells were seeded onto 6 cm dish at 2.6*106/well in 3 ml growth medium supplemented with 10% FBS. After stimulating with F. nucleatum (F.n) lysate 2 ul for 24 hours, the supernatant and cell cytosol were collected. The ratio of microglia to F.n is 2:1. All data are mean±SEM. * p<0.05, **: P<0.01, ****: P<0.0001.

FIG. 11. Targeting SUCNR1 suppressed periodontitis and neuroinflammation. A. Study design. Periodontitis was induced in 14-week-old mice with a 6-0 silk ligature (Roboz Surgical Instrument Co., MD, USA) around the maxillary right side second molar. The ligature were kept in place for 5 days before removal. Sterile swabs soaked in 100 μl of 2.5×10⁹/ml F. nucleatum in PBS and inoculated into mice around the ligature affected gingival tissues for 30 seconds. Inoculations were performed twice a week for four weeks. Mice were randomly assigned to receive 3 μl vehicle or 7a gel formulation every other day by topical application. B. Representative μCT images; C. The distance between CEJ to alveolar bone crest. D. IL-1β mRNA levels in the gingivae around the ligature site. E. Serum IL-1β levels. *p<0.05, ** p<0.01, *** p<0.001).

FIG. 12. Inhibitory effects of 10-week once weekly treatment of 7a gel formulation in vivo. A) Experimental design for panel B and C. B) Representative μCT images and C) quantitative results of maxillae from each group. (N=15, **** p<0.0001). D) TNFα and E) IL1β mRNA levels in the gingival tissues. F) TNFα and G) IL1β protein levels in the serum of each group. (N=8-9, *p<0.05, ** p<0.005, **** p<0.0001).

FIG. 13. Inhibitory effects of 7-day daily treatment of 7a gel formulation in vivo. A) The diagram of the experimental periodontitis model and treatment regime. B) Representative ρCT images of maxillae. Scale bar, 1 mm. and C) Quantitative results of maxillae alveolar bone recession from each group. D) IL-1β protein levels in the indicated brain regions detected using MSD multiplex assay. One-way ANOVA was used for statistical analysis. * P<0.05, ** P<0.01, *** P<0.001.

FIG. 14. The structure of antagonist 4C and other compounds of the present disclosure.

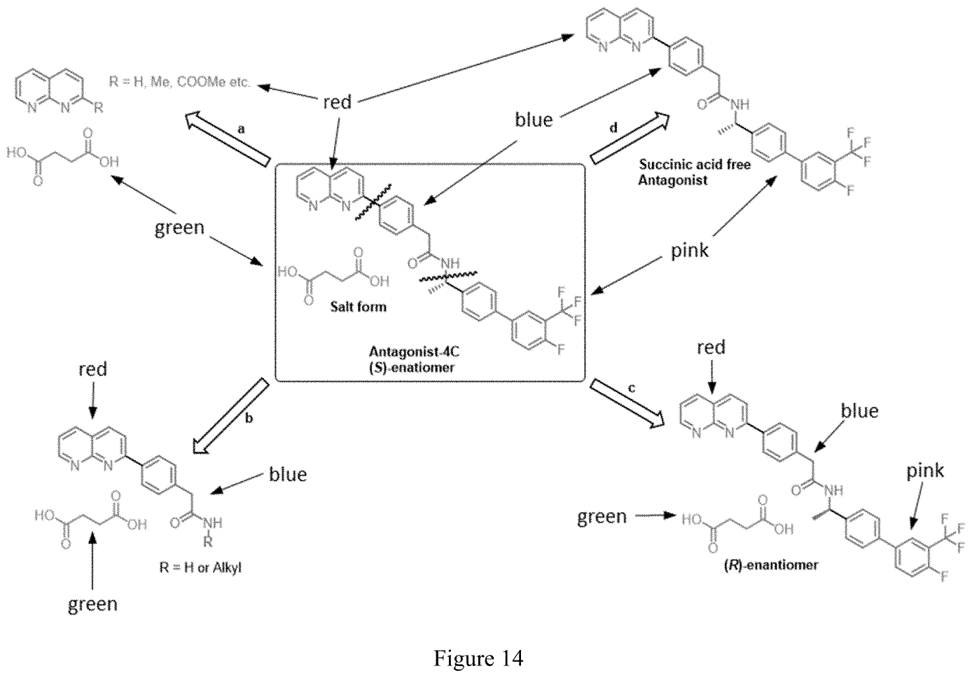

FIG. 15. The structure of compounds 7a, 7b, and 4c free base.

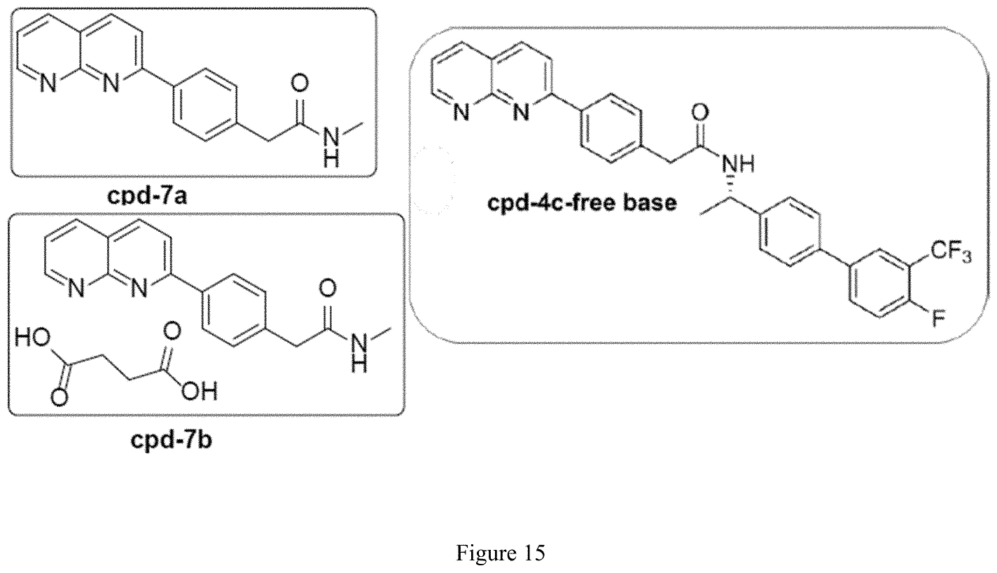

FIG. 16. Oral gavage of SUCNR1 antagonist 7a reduced neuroinflammation in mice. A. Experimental design and treatment regime. 12-week-old male mice were randomly divided into 3 groups to receive inflammation induction (LPS) and treatment (7a) at indicated timepoints. PBS was used as the control solution to LPS and 20% DMSO in PBS (vehicle) was used as the control solution to 7a. The RNA samples were collected in TRIZol and extracted after perfusion. TNFα and IL1β mRNA levels were measured by Realtime PCR using specific primers and normalized to β-actin. B. Cerebellum, C. Hippocampus and D. Cortex. (* p<0.05. ** p<0.005, *** p<0.0005, **** p<0.0001 One-way ANOVA with post-hoc Tukey test).

### DETAILED DESCRIPTION OF THE DISCLOSURE

The present disclose provides methods and compositions/formulations comprising therapeutic agents targeted to interfering with succinate/succinate receptor signaling. These compositions can be used for treatment or prevention of neuroinflammation. The compositions can also be used for veterinary applications.

Whenever a singular term is used in this disclosure, a plural term is also included. For example, “a”, or “an” also includes a plurality of the referenced items, unless otherwise indicated.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the composition used, its mode of administration, patient specifics and the like. Appropriate effective amount can be determined by one of ordinary skill in the art informed by the instant disclosure using only routine experimentation. Treatment can be orientated symptomatically, for example, to suppress symptoms. It can be effected over a short period, over a medium term, or can be a long-term treatment, such as, for example within the context of a maintenance therapy. Treatment can be continuous or intermittent.

Where a range of values is provided in this disclosure, it should be understood that each intervening value, to the tenth of the unit of the lower limit between the upper and lower limit of that range, and any other intervening value in that stated range is encompassed within the invention, unless clearly indicated otherwise. The upper and lower limits of these smaller ranges may independently be included in the smaller ranges encompassed within the disclosure.

It is considered that the formulations of the present disclosure can act to suppress succinate/SUCNR1 activation to alleviate and/or treat neuroinflammation, resulting in amelioration or reduction of neuroinflammation.

Based on one or more of the findings described herein, in one aspect, this disclosure provides a composition (e.g., an oral topical gel or a composition suitable for systemic administration) comprising an inhibitor of the succinate/SUCNR1 pathway.

In an aspect, the present disclosure provides formulations (e.g., oral formulations, such as, for example, gels or compositions suitable for systemic administration) comprising therapeutic agents targeted to interfering with succinate/succinate receptor signaling. For example, the present disclosure provides compositions/formulations (e.g., compositions suitable for systemic administration or oral formulations, such as, for example, gels) comprising succinate/succinate receptor 1 inhibitors. For example, an succinate/succinate receptor 1 inhibitor may be a compound having the following formula:

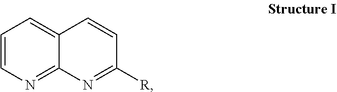

where R is chosen from a hydrogen atom, an alkyl group (e.g., methyl, ethyl, propyl, isopropyl, and the like), a COOR′ group, and

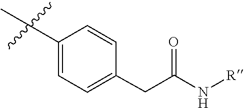

where R′ is an alkyl group (e.g., methyl, ethyl, propyl, isopropyl, and the like), R″ is selected from the group consisting of a hydrogen atom, an alkyl group (e.g., methyl, ethyl, propyl, isopropyl, and the like), and

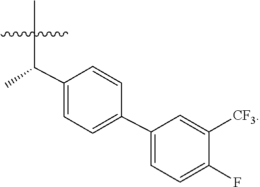 [SMILES: *[C@@H](C)c1ccc(-c2ccc(F)c(C(F)(F)F)c2)cc1]

In various embodiments, the compound is a salt (e.g., a succinate salt).

In an embodiment, the agent interfering with succinate/succinate receptor signaling is a succinate salt of Structure I and may be represented as follows:

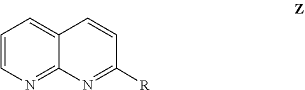

wherein Z is a succinate group.

In an embodiment, the compound interfering with succinate/succinate receptor signaling has the following structure:

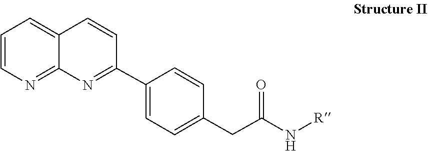

where R″ is selected from the group consisting of a hydrogen atom, an alkyl group (e.g., methyl, ethyl, propyl, isopropyl, and the like), and

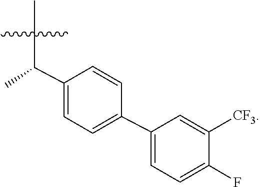 [SMILES: *[C@@H](C)c1ccc(-c2ccc(F)c(C(F)(F)F)c2)cc1]

In an embodiment, the agent interfering with succinate/succinate 1 receptor signaling is a succinate salt of Structure II and may be represented as follows:

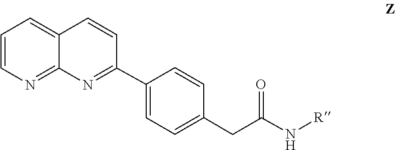

where Z is a succinate group.

In an embodiment, the disclosure provides formulations (e.g., oral gels) comprising compounds 7a (may be referred to herein as cpd 7a or cpd-7a) and/or 7b (may be referred to herein as cpd7a or cpd-7b).

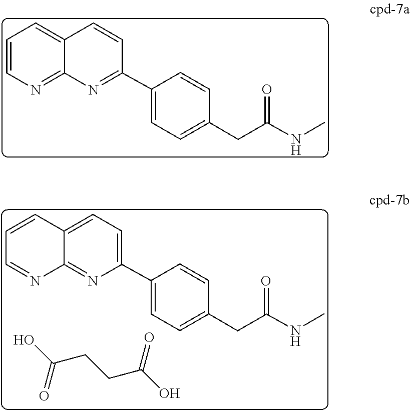

In an embodiment, the compositions may comprise 7a and/or 7b.

In various embodiments, the compositions may comprise any known succinate/succinate receptor 1 inhibitor. For example, the inhibitor may be any compound described herein (e.g., 7a or 7b). In various other examples, the inhibitor is chosen from:

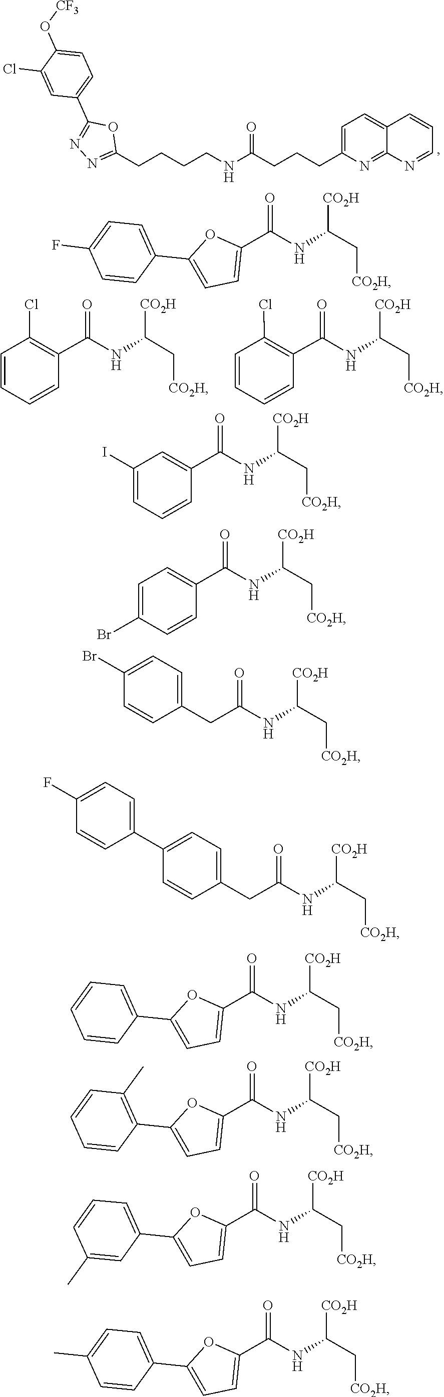

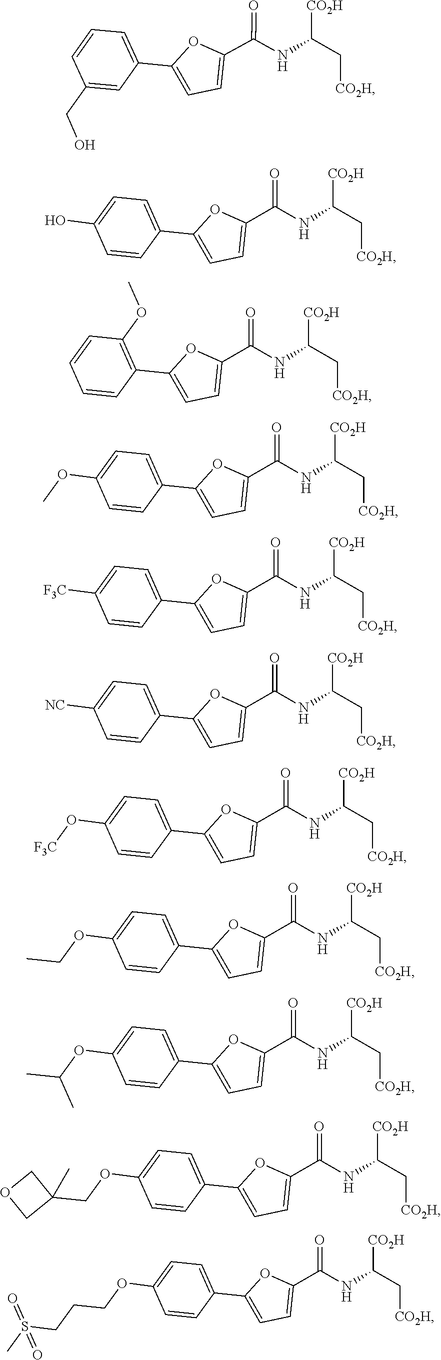

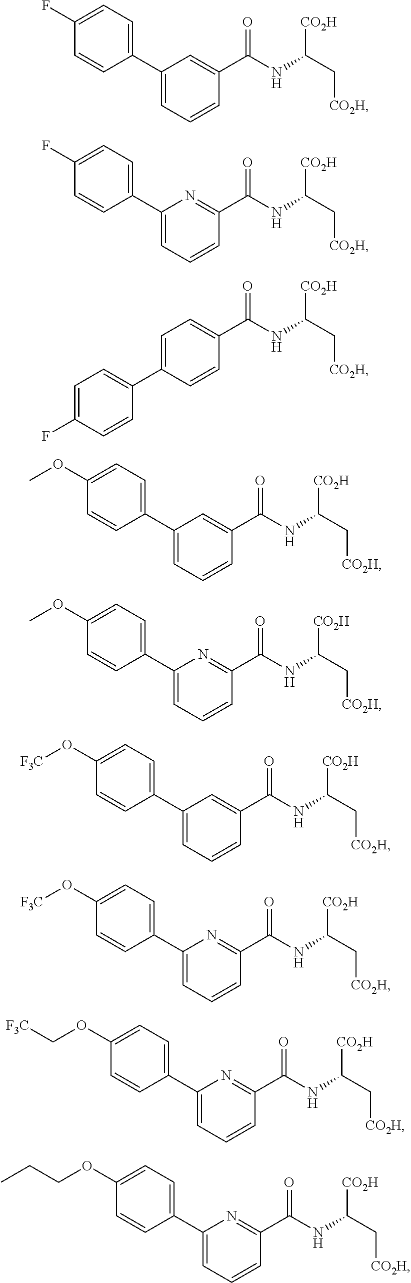

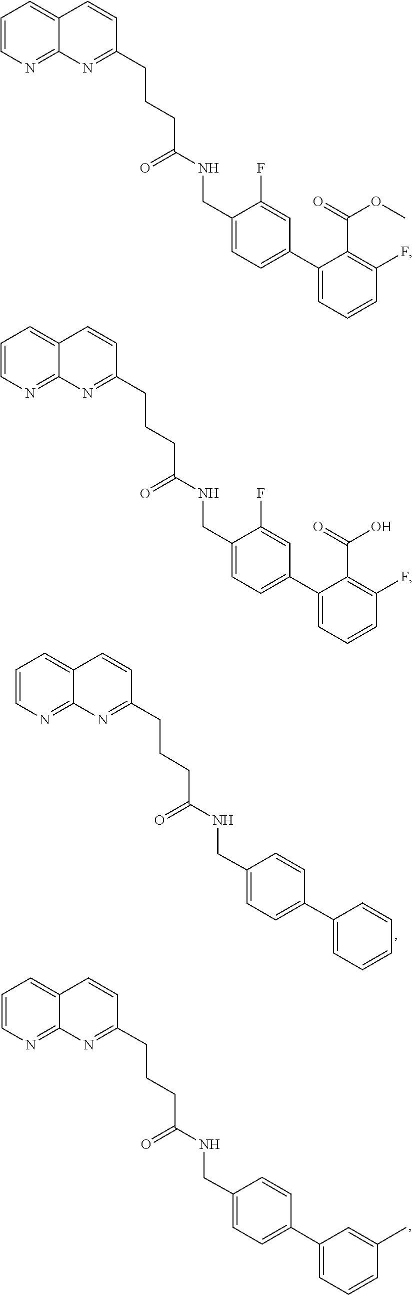 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1]

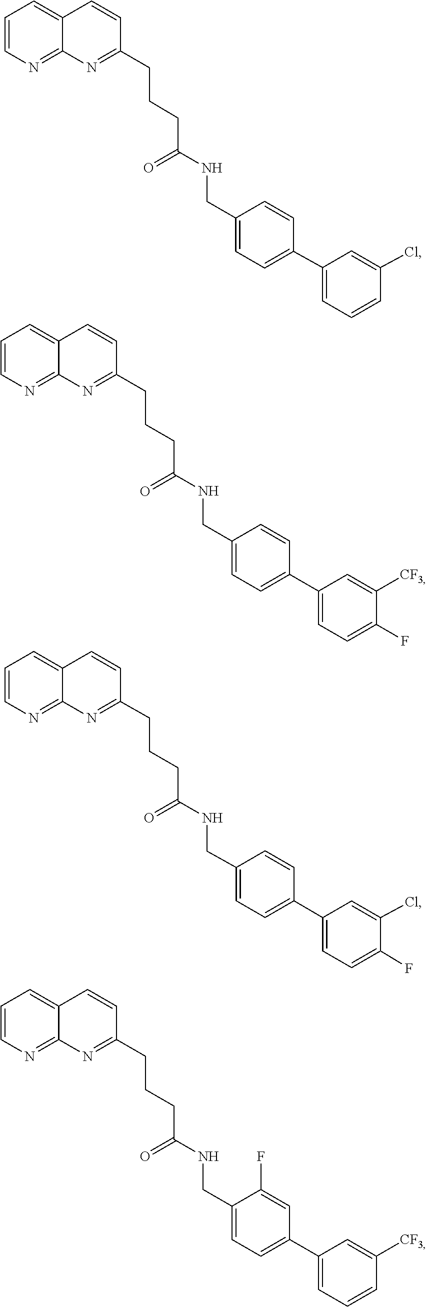 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1 ; O=C(CCCc1ccc2cccnc2n1)NCc1ccc(-c2cccc(C(F)(F)F)c2)cc1F]

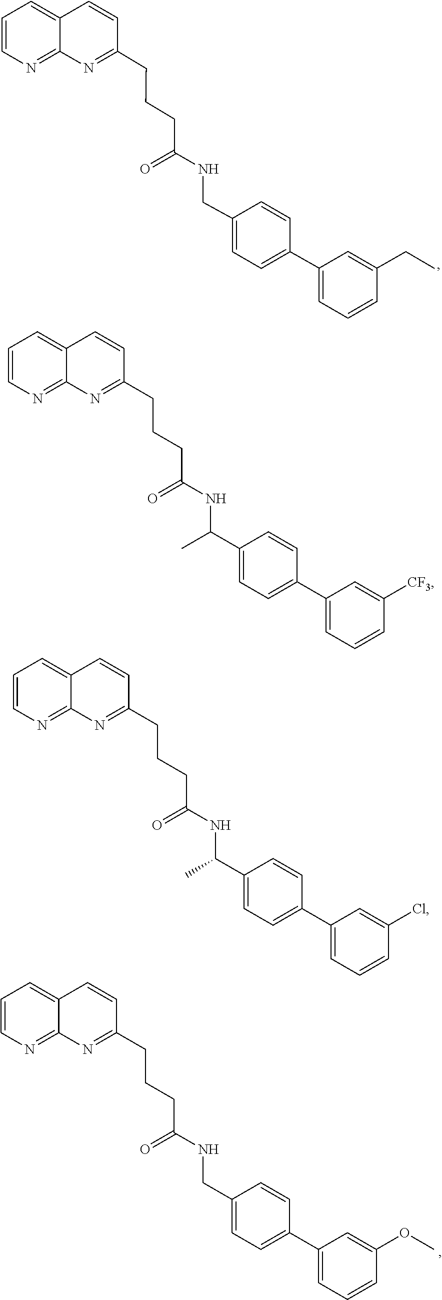 [SMILES: CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1 ; COc1cccc(-c2ccc(CNC(=O)CCCc3ccc4cccnc4n3)cc2)c1]

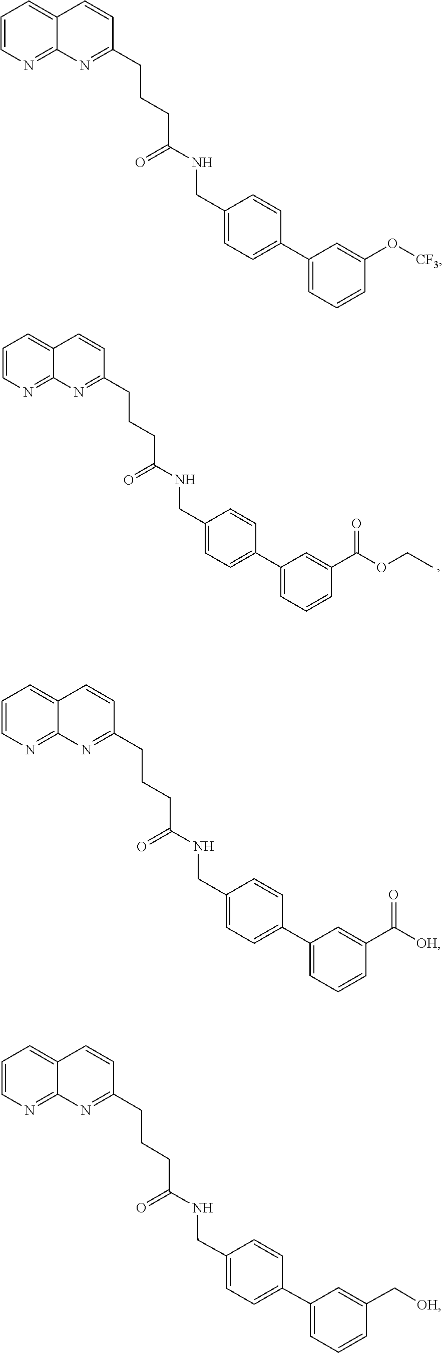 [SMILES: O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1 ; O=C(CCCc1ccc2cccnc2n1)NCc1ccc(-c2cccc(CO)c2)cc1]

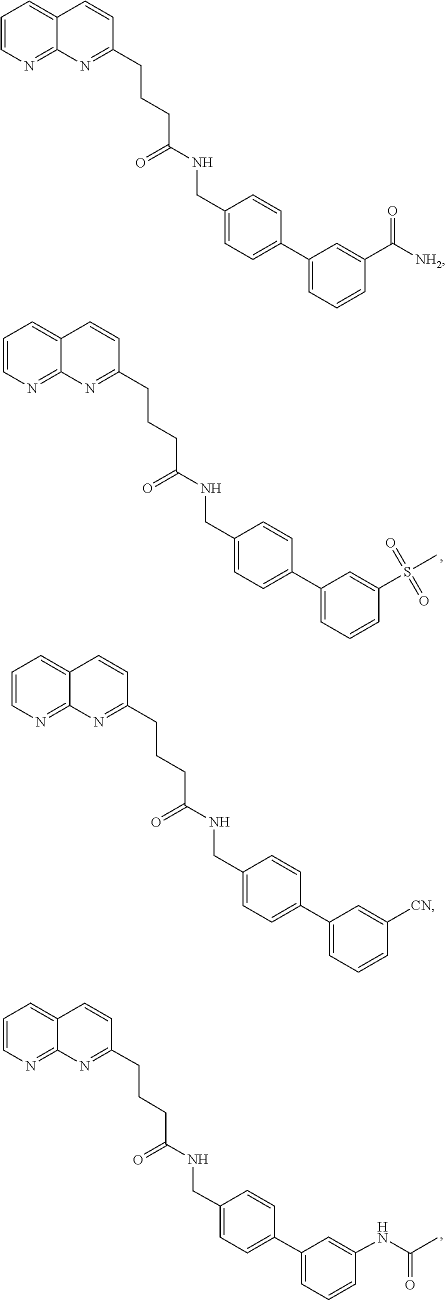 [SMILES: NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1 ; CC(=O)Nc1cccc(-c2ccc(CNC(=O)CCCc3ccc4cccnc4n3)cc2)c1]

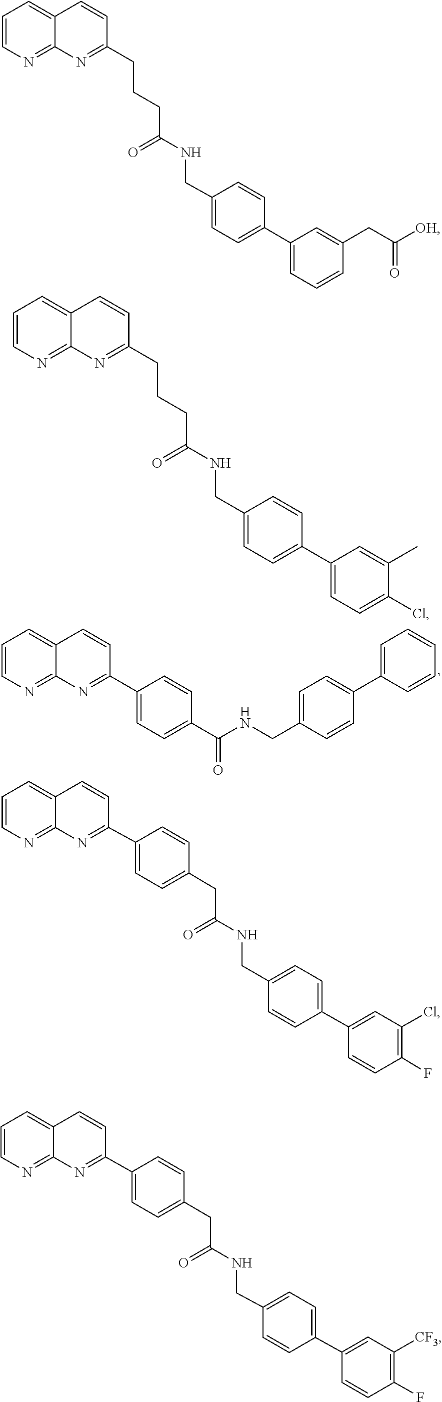

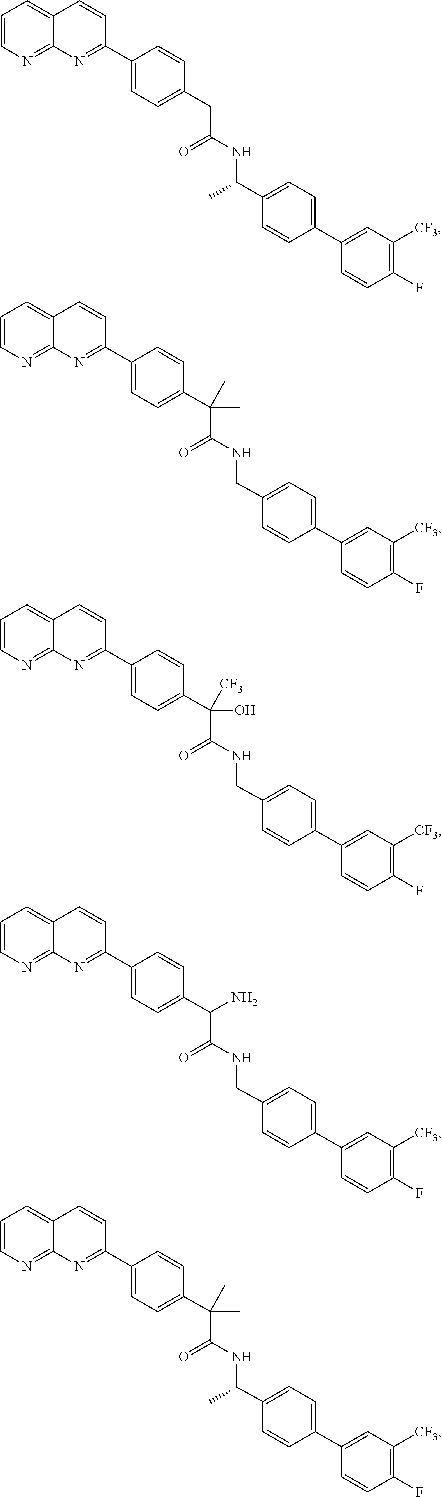 [SMILES: C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1 ; O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1]

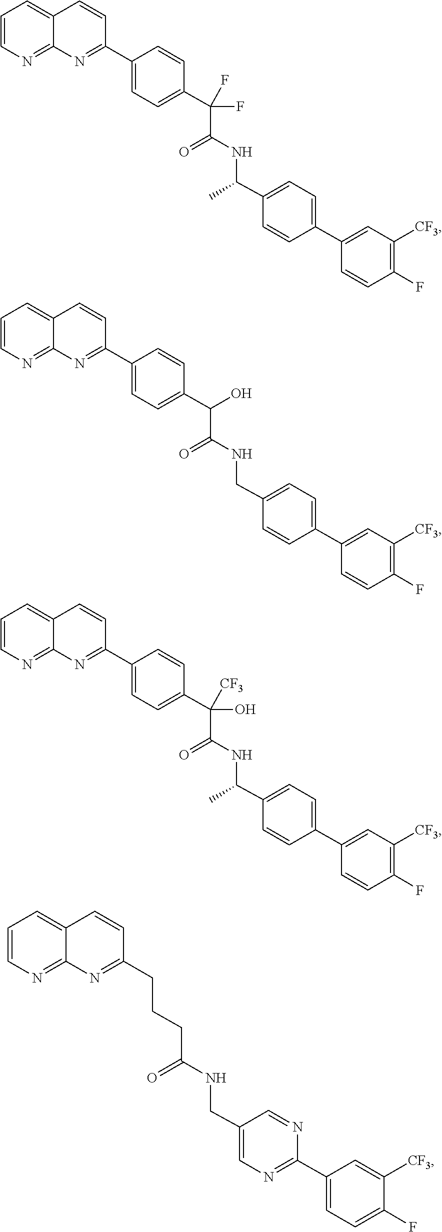 [SMILES: C[C@H](NC(=O)C(F)(F)c1ccc(-c2ccc3cccnc3n2)cc1)c1ccc(-c2ccc(F)c(C(F)(F)F)c2)cc1 ; O=C(NCc1ccc(-c2ccc(F)c(C(F)(F)F)c2)cc1)C(O)c1ccc(-c2ccc3cccnc3n2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1]

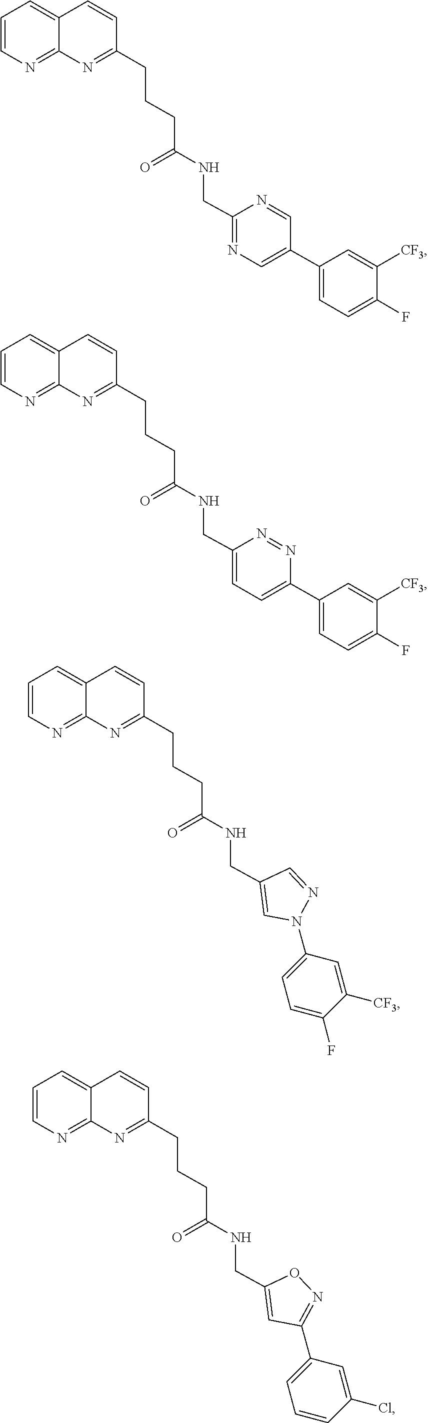 [SMILES: O=C(CCCc1ccc2cccnc2n1)NCc1ncc(-c2ccc(F)c(C(F)(F)F)c2)cn1 ; O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1]

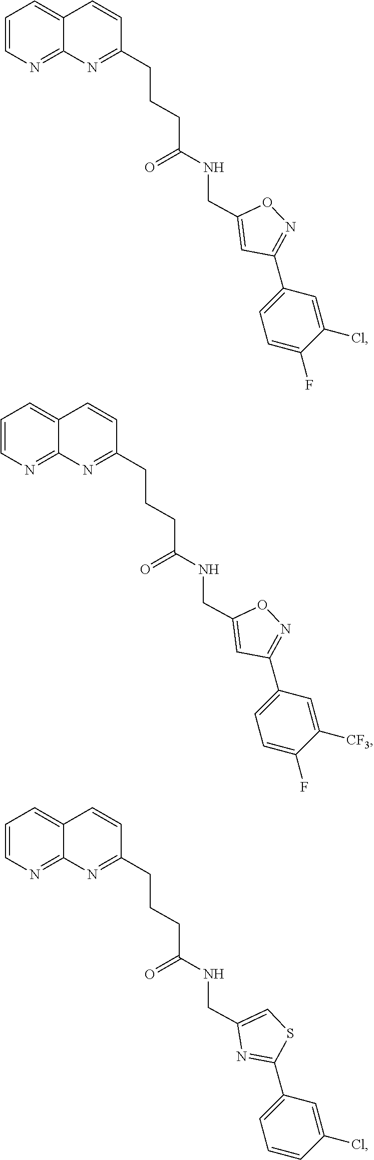 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1]

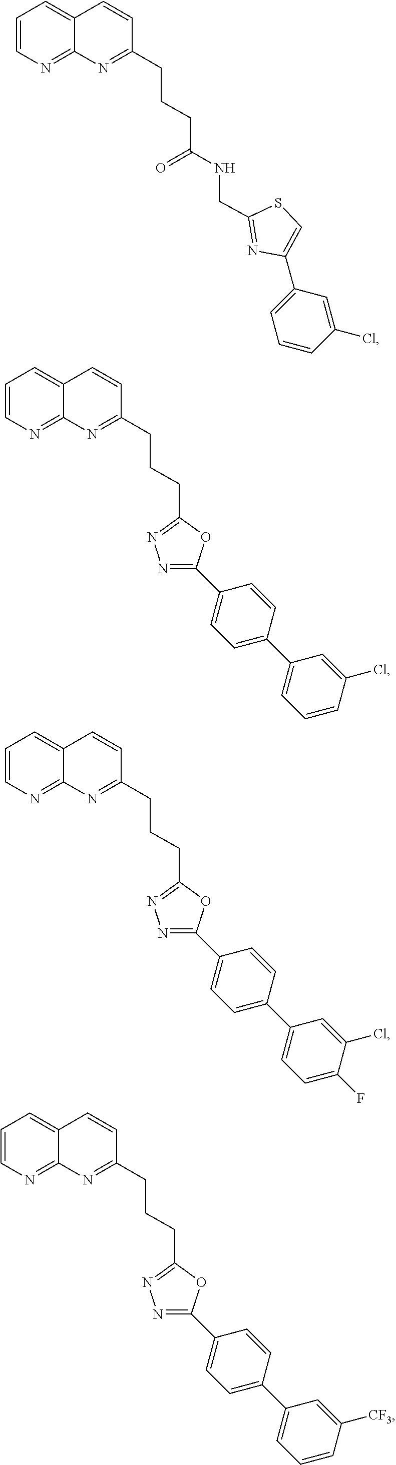 [SMILES: O=C(CCCc1ccc2cccnc2n1)NCc1nc(-c2cccc(Cl)c2)cs1 ; Clc1cccc(-c2ccc(-c3nnc(CCCc4ccc5cccnc5n4)o3)cc2)c1 ; Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1]

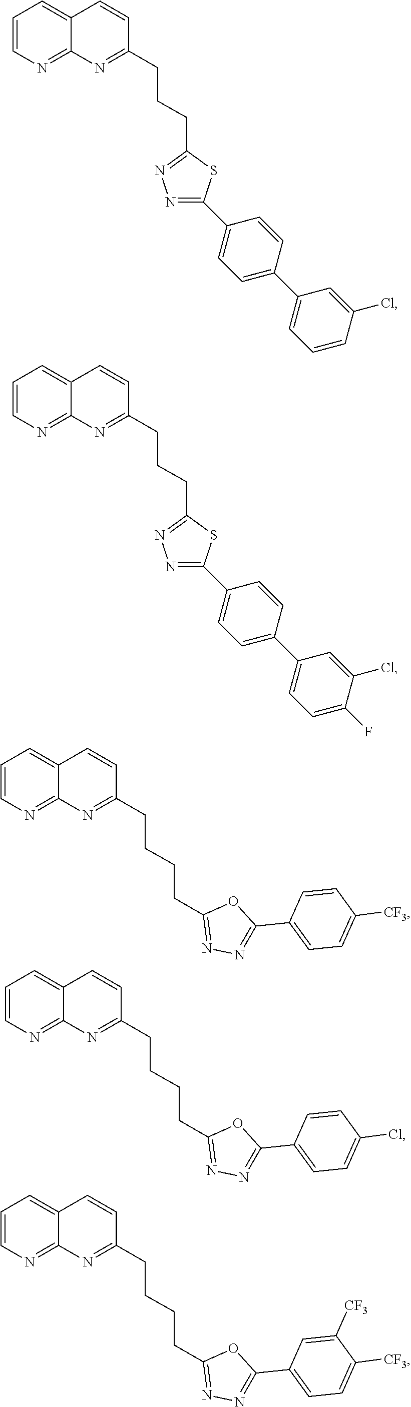 [SMILES: Clc1cccc(-c2ccc(-c3nnc(CCCc4ccc5cccnc5n4)s3)cc2)c1 ; Fc1ccc(-c2ccc(-c3nnc(CCCc4ccc5cccnc5n4)s3)cc2)cc1Cl ; FC(F)(F)c1ccc(-c2nnc(CCCCc3ccc4cccnc4n3)o2)cc1 ; Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F]

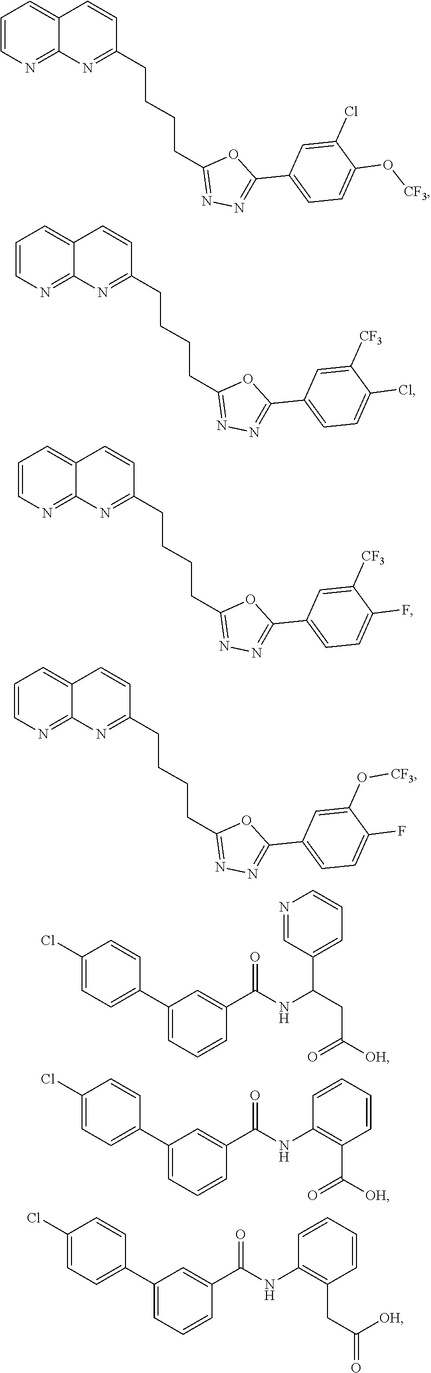

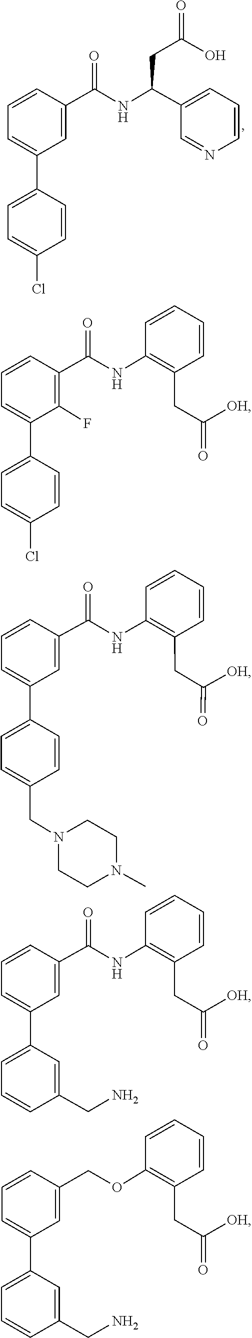

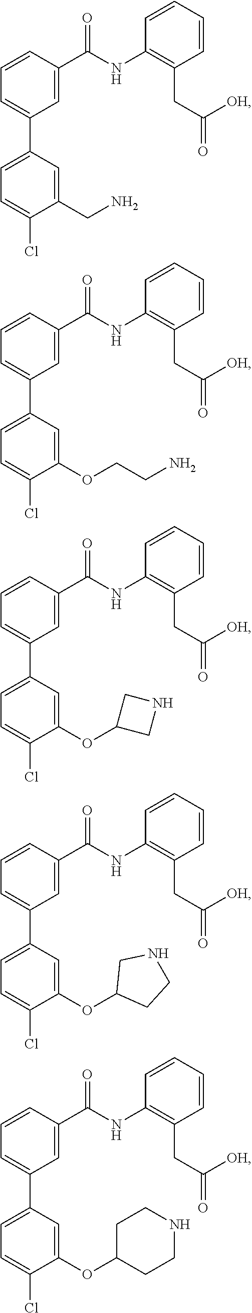 [SMILES: NCc1cc(-c2cccc(C(=O)Nc3ccccc3CC(=O)O)c2)ccc1Cl ; NCCOc1cc(-c2cccc(C(=O)Nc3ccccc3CC(=O)O)c2)ccc1Cl ; O=C(O)Cc1ccccc1NC(=O)c1cccc(-c2ccc(Cl)c(OC3CNC3)c2)c1 ; O=C(O)Cc1ccccc1NC(=O)c1cccc(-c2ccc(Cl)c(OC3CCNC3)c2)c1 ; O=C(O)Cc1ccccc1NC(=O)c1cccc(-c2ccc(Cl)c(OC3CCNCC3)c2)c1]

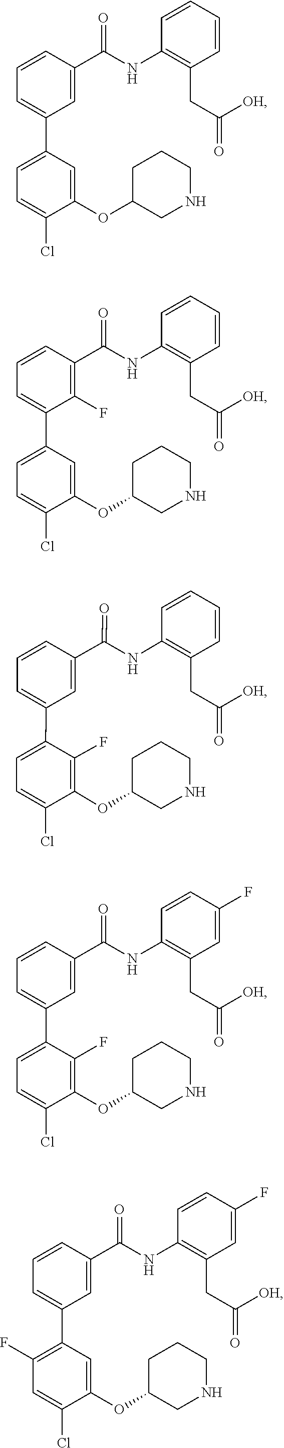 [SMILES: O=C(O)Cc1ccccc1NC(=O)c1cccc(-c2ccc(Cl)c(OC3CCCNC3)c2)c1 ; O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1]

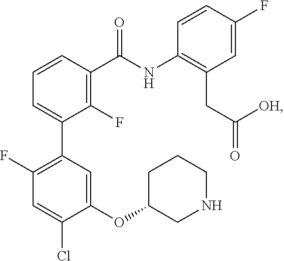 [SMILES: O=C(O)Cc1cc(F)ccc1NC(=O)c1cccc(-c2cc(O[C@@H]3CCCNC3)c(Cl)cc2F)c1F]

and combinations thereof.

In one embodiment, this disclosure provides compositions (e.g., oral gel formulations or compositions suitable for systemic administration) comprising one or more compounds of Structures I, II, 7a, and/or 7b, or any known succinate/succinate 1 receptor inhibitors (including, but not limited to, the inhibitors described here. In an embodiment, the compound is 7a, an inhibitor of SUCNR1.

In an embodiment, the disclosure provides formulations (e.g., compositions suitable for systemic administration or oral gels) comprising compounds 7a (may be referred to herein as cpd 7a or cpd-7a) and/or 7b (may be referred to herein as cpd7a or cpd-7b).

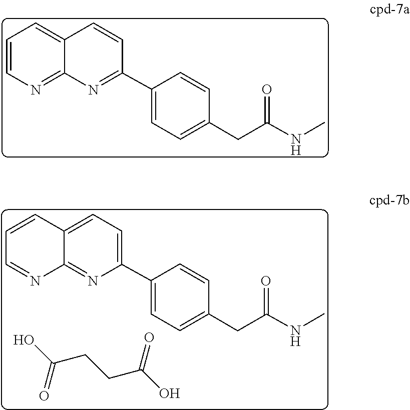

In an embodiment, the compositions may comprise 7a and/or 7b.

The compositions may include one or more pharmaceutically acceptable carrier(s). Non-limiting examples of compositions include solutions, suspensions, emulsions, solid injectable compositions that are dissolved or suspended in a solvent before use, and the like. Injections may be prepared by dissolving, suspending, or emulsifying one or more of the active ingredient(s) in a diluent. Non-limiting examples of diluents include distilled water (e.g., for injection), physiological saline, vegetable oil, alcohol, and the like, and combinations thereof. Injections may contain, for example, stabilizers, solubilizers, suspending agents, emulsifiers, soothing agents, buffers, preservatives, and the like, and combinations thereof. Injections may be sterilized in the final formulation step or prepared by sterile procedure. A pharmaceutical composition of the disclosure may also be formulated into a sterile solid preparation, for example, by freeze-drying, and may be used after sterilized or dissolved in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, such as, for example, sodium carboxymethyl cellulose, ethyl cellulose, and cellulose acetate; powdered tragacanth; malt; gelatin; talc; excipients, such as, for example, cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example, glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; other non-toxic compatible substances employed in pharmaceutical formulations, and the like, and combinations thereof. Non-limiting examples of pharmaceutically acceptable carriers are found in: Remington: The Science and Practice of Pharmacy (2012) 22nd Edition, Philadelphia, PA. Lippincott Williams & Wilkins.

Compositions of the disclosure can comprise more than one pharmaceutical agent. For example, a first composition comprising a compound of the disclosure and a first pharmaceutical agent can be separately prepared from a composition which comprises the same compound of the disclosure and a second pharmaceutical agent, and such preparations can be mixed to provide a two-pronged (or more) approach to achieving the desired prophylaxis or therapy in an individual. Further, compositions of the disclosure can be prepared using mixed preparations of any of the compounds disclosed herein.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In various embodiments, the composition is a film, a gel, a capsule, a tablet, a plurality of nanoparticles, or liquid composition. When the inhibitor has the following structure:

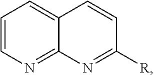

where R is selected from the group consisting of a hydrogen atom, an alkyl group, COOR′ group, and

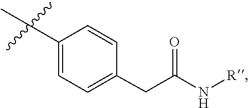

where R′ is an alkyl group and R″ is selected from the group consisting of a hydrogen atom, an alkyl group, and

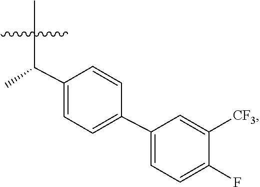 [SMILES: *[C@@H](C)c1ccc(-c2ccc(F)c(C(F)(F)F)c2)cc1]

the composition is not a thin film having a thickness of from 0.05 mm to 0.4 mm.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a compound of the present disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to a compound of the disclosure, the composition may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The composition may be for administration to an individual in need of treatment.

When the composition comprises a gel, the gel may comprise various polymers or gel precursors. For example, the polymers may be polyesters or polyamides. In various embodiments, the polymers are biodegradable polymers. Examples of such polymers include, but are not limited to poly lactic glycolic acid (PLGA) (e.g., having an M_{w} of 50,000 to 75,000 Da), polycaprolactone (PCL) (e.g., poly-ε-caprolactone ester terminated) (e.g., having an M_{w} of 14,000 Da), poly(D,L-lactide-co-glycolide) (which may be ester terminated), poly(D,L-lactide) (which may be ester terminated) (e.g., having an M_{w} of 50,000-75,000 Da), chitosan, starch, polylactic acid, alginate, and the like, and combinations thereof. Additional examples of polymers include, but are not limited to, polylactides (PLA), polyglycolides (PGA), polyanhydrides, polyorthoesters, and the like.

In various examples, the polymers may have a desirable intrinsic viscosity. For example, PLGA has an intrinsic viscosity of 0.55-0.75 dL/g in hexafluoroisopropanol (HFIP) or an intrinsic viscosity of 0.26-0.54 dL/g, poly(D,L-lactide-co-glycolide) has an intrinsic viscosity of 0.55-0.75 dL/g in CHCCl₃, and poly-ε-caprolactone ester terminated has an intrinsic viscosity of 0.65-0.85 dL/g in CHCl₃. In various examples, one or more polymers may be used to prepared the gel.

Various concentrations of the polymers and/or gel precursors may be used. For example, the concentration of polymer and/or gel precursor is such that a gel of desirable rigidity is produced. For example, the concentration of polymer and/or gel precursor (e.g., total concentration of polymer and/or gel precursor) may be 1-50% w/v, including all 0.1 values and range therebetween (e.g., 5%, 10%, 15%, 20%, or 25% w/v).

Various solvents may be used to solvate the polymers or gel precursors. In various examples, the one solvent or a mixture of solvents is used. In various examples, the solvent is an organic solvent. The solvent may be a solvent suitable to solvate the polymers or gel precursors described herein. Non-limiting examples of solvents include, benzyl benzoate (BB), N-methyl-2-pyrrolidone (NMP), and the like, and combinations thereof.

Various ratios of solvents may be used. For example, various ratios of BB to NMP may be used. For example, the volume ratio of BB to NMP is 10:1 to 1:10, including all ratio values therebetween (e.g., 7:3, 1:1, 1:4).

In an embodiment, the only compounds in the composition interfering with succinate/succinate receptor signaling are the compounds of Structure I, Structure II, 7a, and/or 7b. In an embodiment, the compounds interfering with succinate/succinate receptor signaling (such as compounds of Structure I, Structure II, succinate salts thereof, 7a, and/or 7b) are used in the present gels at a desirable purity, such as being at least 80%, 85%, 90%, 95%, 96%, 97%, 98%, 99% or 100% pure—as verified by analytical methods, such as HPLC. In other embodiments, the compound is a known succinate/succinate receptor inhibitor other than 7a and/or 7b or a compound of Structure I or Structure II.

The concentration of compounds of Structure I, Structure II, 7a, and/or 7b in the composition may be enough to reduce neuroinflammation. This amount may be referred to as “therapeutically effective amount.” For example, the concentration of 7a and/or 7b in a gel composition is 1-10 mg/mL, including all 0.01 mg/mL values and ranges therebetween (e.g., 5.0 mg/mL) (e.g., 1, 2, 3, 4, 5, 6, 7, 8, 9, or 10 mg/mL). In various embodiments, the therapeutically effective amount is 1 μg/10 g body weight of 7a or 7b.

The compositions/formulations (e.g., gels or compositions suitable for systemic administration) may additionally comprise one or more of the following optional components: taste modifiers, bioadhesive agents, buffering agents, coloring agents, stabilizers, inert fillers, emulsifiers, pH adjusting agents, plasticizers, preservatives, and any other agent useful for release or stability of the active agent(s). Examples of plasticizers include, but are not limited to PEG (e.g., PEG200). Various concentrations of plasticizers may be used. For example, the concentration of the plasticizer is 1-10%, including all values and ranges therebetween (e.g., 3.5% PEG200).

Suitable taste modifiers include flavorants, sweeteners, and taste-masking agents. Examples of taste modifying agents include, but are not limited to, the essential oils or water soluble extracts of menthol, wintergreen, peppermint, sweet mint, spearmint, vanilla, cherry, butterscotch, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, green tea, grapefruit, banana, butter and the like. Sweeteners (including artificial sweeteners) include sugar, honey, dextrose, lactose, aspartame, saccharin, sodium cyclamate, and acesulfame K.

Suitable colorants include, but are not limited to, pigments, dyes, natural food colors that are suitable for food and drug applications, such as any colorants approved by the FDA for food products and oral composition products, including dental products.

The compositions/formulations (e.g., gels) may also comprise inert fillers such as mannitol, xylitol, glucose, fructose, sucrose, sucralose, lactose, trehalose, maltodextrin, dextran, dextrin, modified starches, dextrose, sorbitol, dextrates, and mixtures thereof.

Suitable emulsifiers include castor oil derivatives, cetyl alcohol, ethanol, hydrogenated vegetable oils, polyvinyl alcohol, simethicone, sorbitan ester, glyceryl monostearate, polyoxyethylene alkyl ethers, polyoxyethylene stearates, poloxamer, polysorbates, and mixtures thereof. Other suitable additives include plasticizers, such as, alkylene glycols, polyalkylene glycols, glycerol, triacetin, deacetylated monoglyceride, diethyl salate, triethyl citrate, dibutyl sebacate, polyethylene glycols, and the like, and mixtures thereof.

The compositions/formulations (e.g., gels or compositions suitable for systemic administration) may also include one or more preservatives, such as, butylated hydroxyanisole (BHA), butylate hydroxytoluene (BHT), ascorbic acid, tocopherol derivatives, citric acid, parabens, derivatives of parabens, sorbic acid, salts of sorbic acid, sodium benzoate, propionic acid, salts of propionic acid, acetic acid, salts of acetic acid and the like.

Gel compositions can generally be prepared by dissolving the active agent in a suitable solvent, and mixing together with a gel forming polymer, a compatible solvent, and optionally any one or more of the optional additives to form a mixture, which may be homogenous. The mixtures can then be optionally heated to facilitate solvation of polymers and/or gel precursors, and allowed to stand and rigidify. The gel can be made into desired shapes and sizes. As an example, the compounds of the present disclosure (such as 7a and/or 7b) may be used at a concentration of from 5 to 50 μM.

The viscosity of the in situ gel formulations can be from 2200 to 3200 cps. For example, in an embodiment, the viscosity of an in situ gel formulation measured at 37° C. was found to be 2304-3030.53±1.92 cps. Upon placement into the periodontal pockets or on gums, it is in contact with physiological pH and temperature 37° C. resulting in the formation of stiff gel with high strength and mucoadhesion properties. In an embodiment, the gels can further comprise Poloxamer 407 (5-20% w/v) and Carbopol 934P (5-25%).

In various examples, the composition may be suitable for injection. Parenteral administration includes infusions and injections, such as, for example, intramuscular, intravenous, intraarterial, intraperitoneal, subcutaneous administration, and the like.

The compositions may be administered systemically. The term “systemic” as used herein includes parenteral, topical, oral, spray inhalation, rectal, nasal, and buccal administration. The term “parenteral” as used herein includes subcutaneous, intravenous, intramuscular, intra-articular, intra-synovial, intrasternal, intrathecal, intrahepatic, intralesional, and intracranial administration. Compositions may be administered orally, may be administered parenterally, and/or intravenously. Compositions suitable for parenteral, administration may include aqueous and/or non-aqueous carriers and diluents, such as, for example, sterile injection solutions. Sterile injection solutions may contain anti-oxidants, buffers, bacteriostatic agents and solutes, which render the composition isotonic with the blood of the intended recipient. Aqueous and/or non-aqueous sterile suspensions may include suspending agents and thickening agents.

Nasal aerosol and inhalation compositions of the present disclosure may be prepared by any method in the art. Such compositions may include dosing vehicles, such as, for example, saline; preservatives, such as, for example, benzyl alcohol; absorption promoters to enhance bioavailability; fluorocarbons used in the delivery systems (e.g., nebulizers and the like; solubilizing agents; dispersing agents; or a combination thereof).

Compositions suitable for systemic administration are also disclosed. For example, an inhibitor as described herein may be used to prepare a stock solution (e.g., a 100 mM stock solution in DMSO). The stock solution may be further diluted with PBS to a concentration suitable for administration (e.g., 1-20 mM of inhibitor). Various solvents may be used to prepare the stock solution. Such solvents are described herein and are known in the art.

In an aspect, this disclosure provides a method of treating of inflammation in an individual, whereby the inflammation is reduced. The inflammation may be neuroinflammation. The method may comprise administering to an individual in need of treatment a composition comprising a succinate/succinate receptor 1 inhibitor. The composition is administered to a location other than the brain of the individual. In various embodiments, when the inhibitor has the following structure:

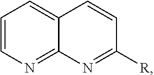

where R is selected from the group consisting of a hydrogen atom, an alkyl group, COOR′ group, and

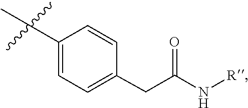

where R′ is an alkyl group and R″ is selected from the group consisting of a hydrogen atom, an alkyl group, and

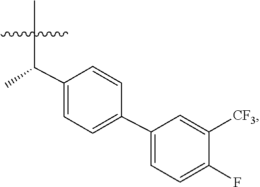 [SMILES: *[C@@H](C)c1ccc(-c2ccc(F)c(C(F)(F)F)c2)cc1]

the composition is not a thin film having a thickness of from 0.05 mm to 0.4 mm. The present disclosure also provides a method to reduce cytokine production, thereby preventing cytokine induced inflammation (e.g., neuroinflammation). The method reduces the expression of pro-inflammatory cytokines (e.g., TNFα, IL1β, or a combination thereof). The method may comprise administering to an individual in need of treatment a composition comprising a succinate/succinate receptor 1 inhibitor. The composition is administered to a location other than the brain of the individual. The composition may be administered topically. The composition may be administered systemically. The method may further comprise identifying an individual with neuroinflammation or identifying an individual with a disease that may result in or cause neuroinflammation.

A method of the present disclosure results in reduction of inflammation. For example, the method results in reduction of local inflammation and/or peripheral inflammation. In various other examples, the method results in reducing of systemic or chronic inflammation.

Administration of the composition may occur by any route. In various embodiments, the administration is via topical application. The topical application may be topical application in the oral cavity of an individual, such as, for example, on the periodontal tissue of the individual. In other embodiments, the administration is systemic. Systemic administration is described herein.

The neuroinflammation may be caused by a disease, a disorder, an infection, an injury, an immune response, or by aging. A method of the present disclosure may reduce neuroinflammation caused by any of the preceding. In various examples, the neuroinflammation is caused by a central nervous system disease or disorder. A method of the present disclosure may be used to reduce or ameliorate such neuroinflammation.

Various infections may result in neuroinflammation. For example, the infection could be caused by or associated with a virus, bacterium, fungus, protozoa, parasite, the like, or combinations thereof. In various embodiments, the infection may result in encephalitis, meningitis or the like, which may cause neuroinflammation. A method of the present disclosure may be used to reduce or ameliorate such neuroinflammation.

Various diseases may result in neuroinflammation or may cause neuroinflammation. Examples of diseases include, but are not limited to, acute disseminated encephalomyelitis, acute optic neuritis, transverse myelitis, neuromyelitis optica, the like, and combinations thereof. In various examples, the disease is a neurodegenerative disorder. Examples of neurodegenerative disorders include, but are not limited to, amyotrophic lateral sclerosis (ALS), Alzheimer's Disease (AD), Parkinson's Disease (PD), multiple sclerosis (MS), the like, and combinations thereof. A method of the present disclosure may be used to reduce or ameliorate such neuroinflammation.

Various psychiatric disorders may result in neuroinflammation and vice versa. Examples of psychiatric disorders include, but are not limited to, schizophrenia, autism, depression, mood disorders, the like, and combinations thereof. A method of the present disclosure may be used to reduce or ameliorate such neuroinflammation.

Additional examples of conditions and pathologies that result in or are associated with neuroinflammation, include, but are not limited to, Adrenal Leukodystrophy (ALD), Alcoholism, Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia, Spinal muscular atrophy, Steele-Richardson-Olszewski disease, Tabes dorsalis, Toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, Hereditary Spastic Paraparesis, Mitochondrial myopathy, periodontal disease, Friedreich Ataxia, the like, and combinations thereof. In various other embodiments the disease/disorder/pathology may be the result of a mutation Succinate dehydrogenase (SDHx) genes (SDHA, SDHB. SDHC. and SDHD),

In an example, an individual can be a human or a non-human subject. Non-limiting examples of non-human subjects includes domestic animals (e.g., dogs, cats, and the like), farm animals (e.g., horses, pigs, and the like), dairy animals (e.g., cows and the like), and the like.

The present compositions can be controlled and sustained drug release formulations for neuroinflammation including dental gels, pills, and capsules. The formulations can comprise biodegradable polymers, which may be natural or synthetic. For example, the polymers may be one or more of chitosan, gellan gum, pectin (natural polymers) and Carbopol 974P, Hydroxy propyl methyl cellulose (HPMC), Poloxamers, methyl cellulose (synthetic/semi-synthetic gelling polymers). Gel formulations of the present disclosure can be prepared by standard methods, which are described herein.

The amount of the antagonist (also referred to herein as the inhibitor) and the vehicle (e.g., polymer) can be varied to provide desired activity, release, and other characteristics. For example, the antagonist to polymer ratio (w/w) can be 0.01:100 to 1:100.

In an example, the antagonist to polymer ratio is 1:50. In one example, polymers, such as gelatin, chitosan or cellulose based biodegradable polymer vehicles are present at from 4-16% concentration.

The concentration of therapeutic agent (e.g., 7a, 7b, or combination thereof) can be from 1-200 μM, including all 0.1 μM values and ranges therebetween. In embodiments, the concentration of the antagonist is 5 μM, 10 μM, 20 μM, 50 μM, 100 μM, 150 μM or 160 μM. Other therapeutic agents (e.g., SUCNR/SUCNR1 receptor inhibitors) may be used in a method of the present disclosure at the aforementioned concentrations. Examples of such inhibitors include, but are not limited to,

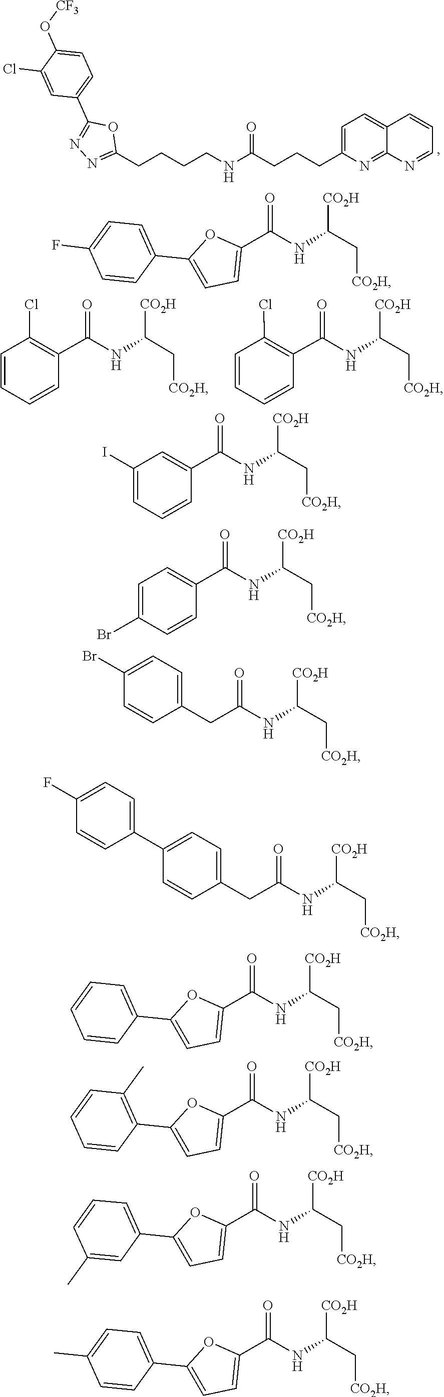

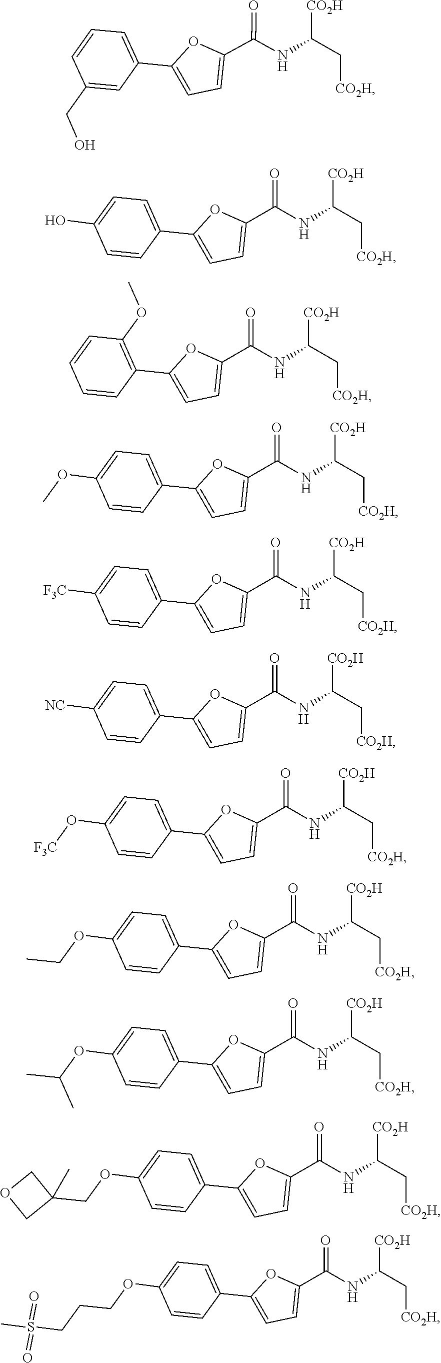

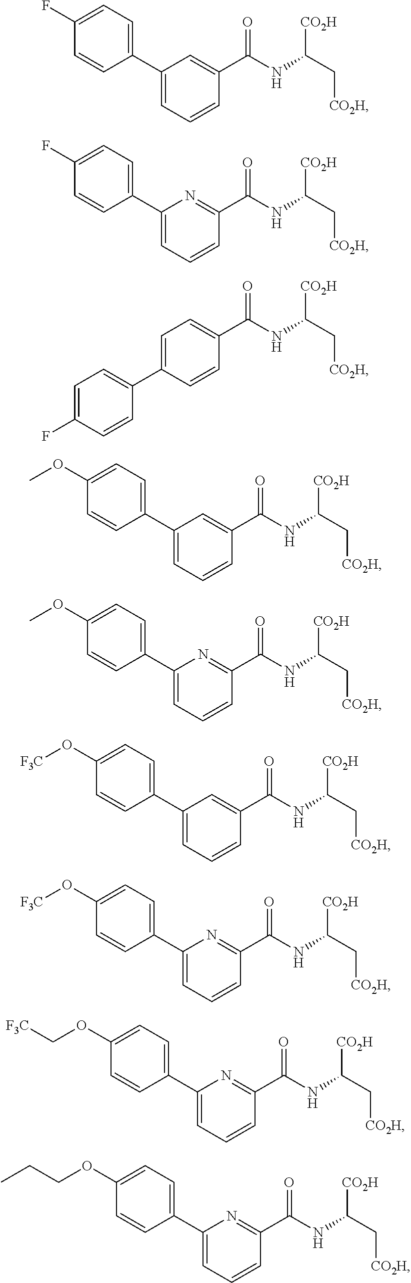

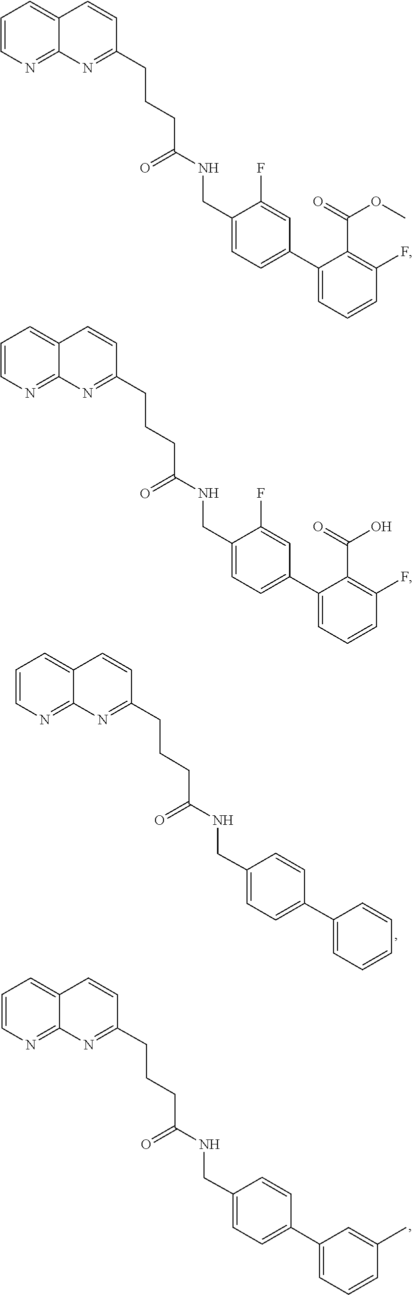 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1]

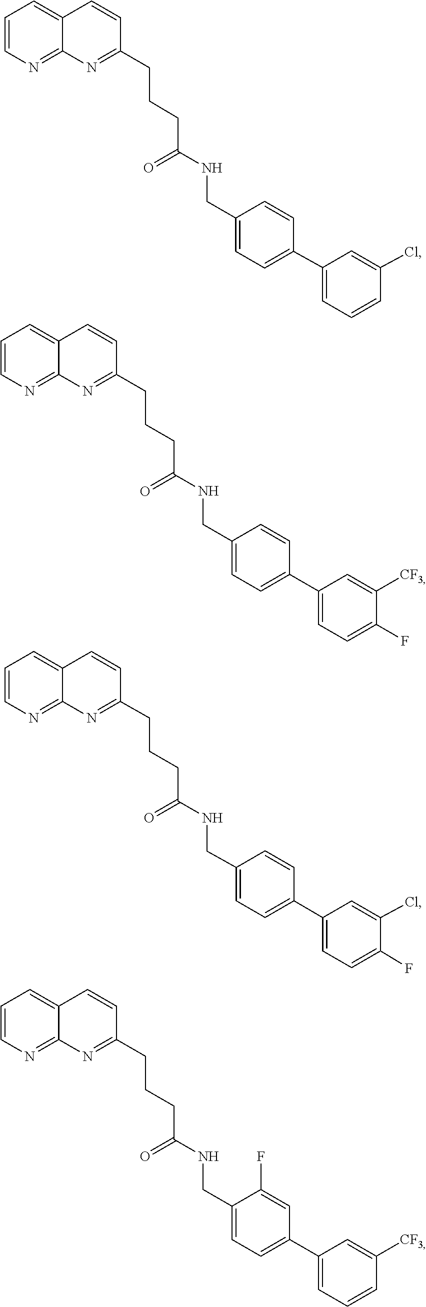 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1 ; O=C(CCCc1ccc2cccnc2n1)NCc1ccc(-c2cccc(C(F)(F)F)c2)cc1F]

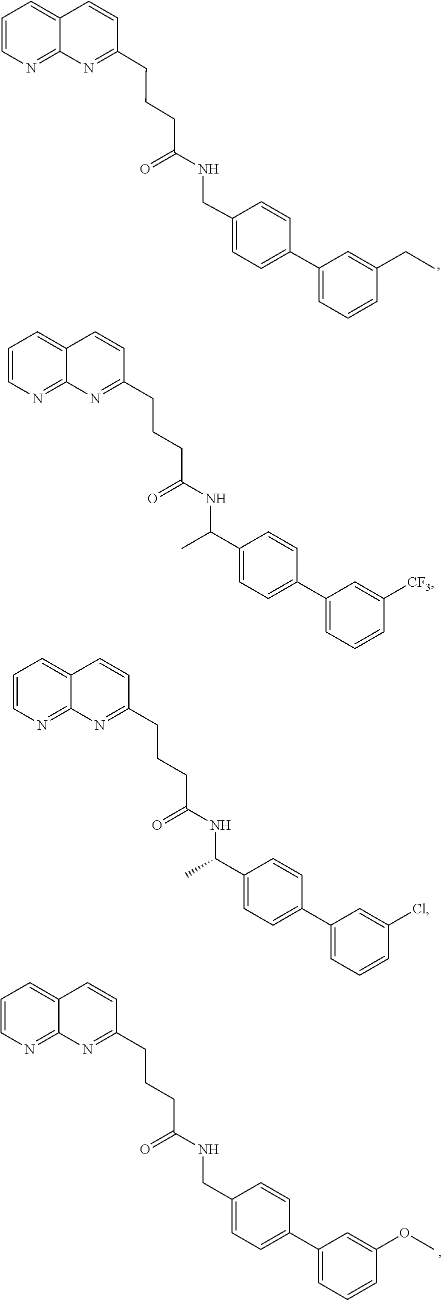 [SMILES: CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1 ; COc1cccc(-c2ccc(CNC(=O)CCCc3ccc4cccnc4n3)cc2)c1]

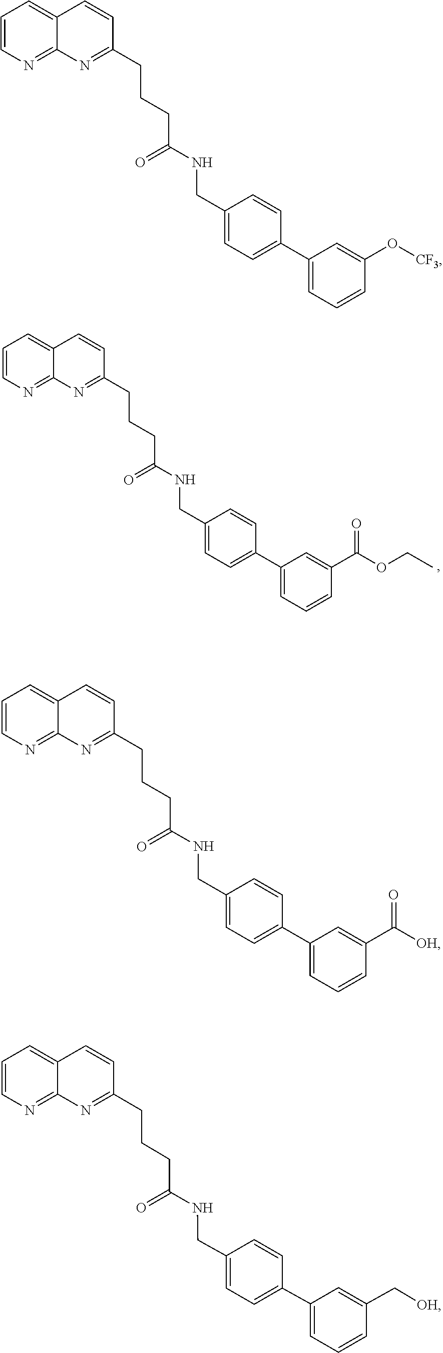 [SMILES: O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1 ; O=C(CCCc1ccc2cccnc2n1)NCc1ccc(-c2cccc(CO)c2)cc1]

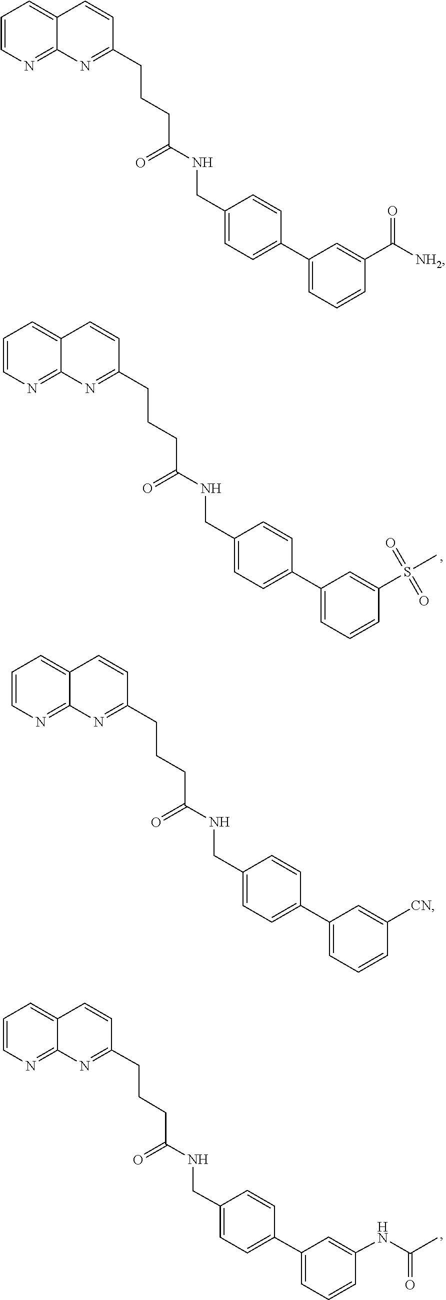 [SMILES: NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1 ; CC(=O)Nc1cccc(-c2ccc(CNC(=O)CCCc3ccc4cccnc4n3)cc2)c1]

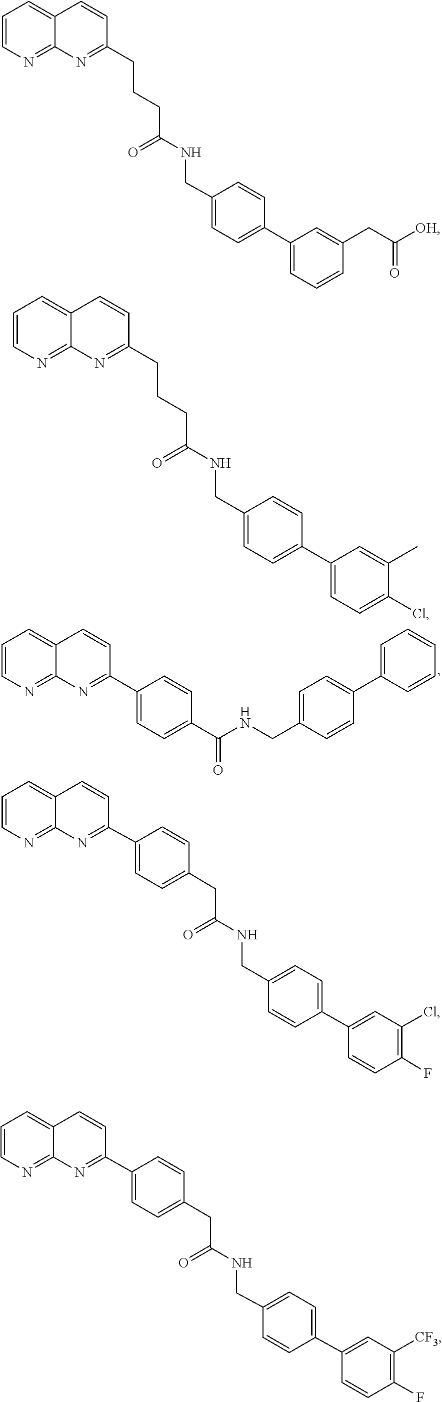

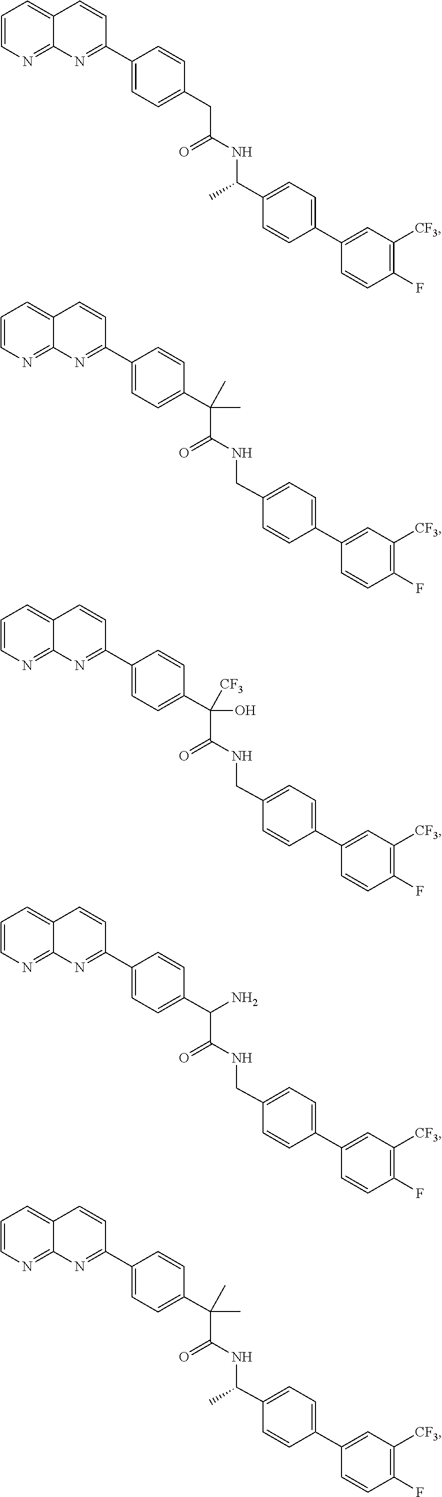 [SMILES: C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1 ; O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1]

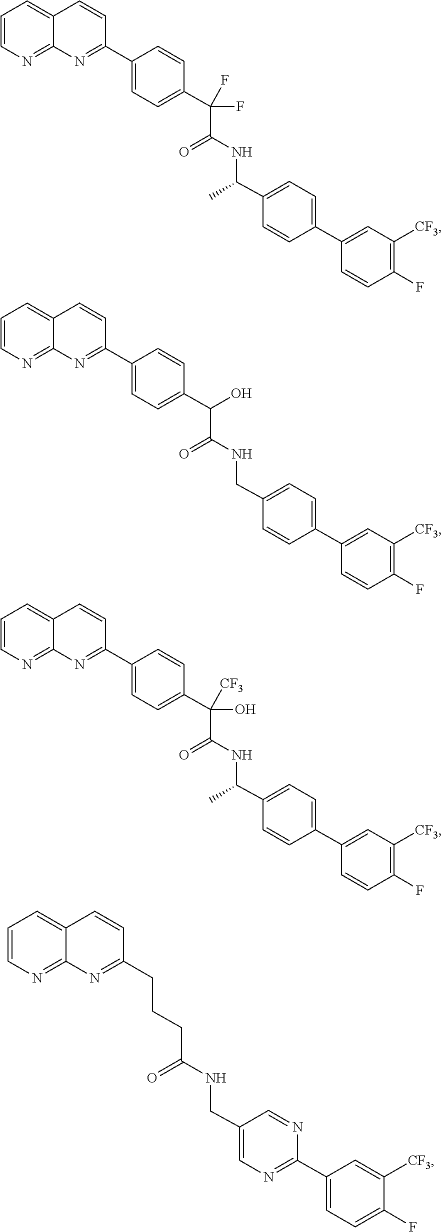 [SMILES: C[C@H](NC(=O)C(F)(F)c1ccc(-c2ccc3cccnc3n2)cc1)c1ccc(-c2ccc(F)c(C(F)(F)F)c2)cc1 ; O=C(NCc1ccc(-c2ccc(F)c(C(F)(F)F)c2)cc1)C(O)c1ccc(-c2ccc3cccnc3n2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1]

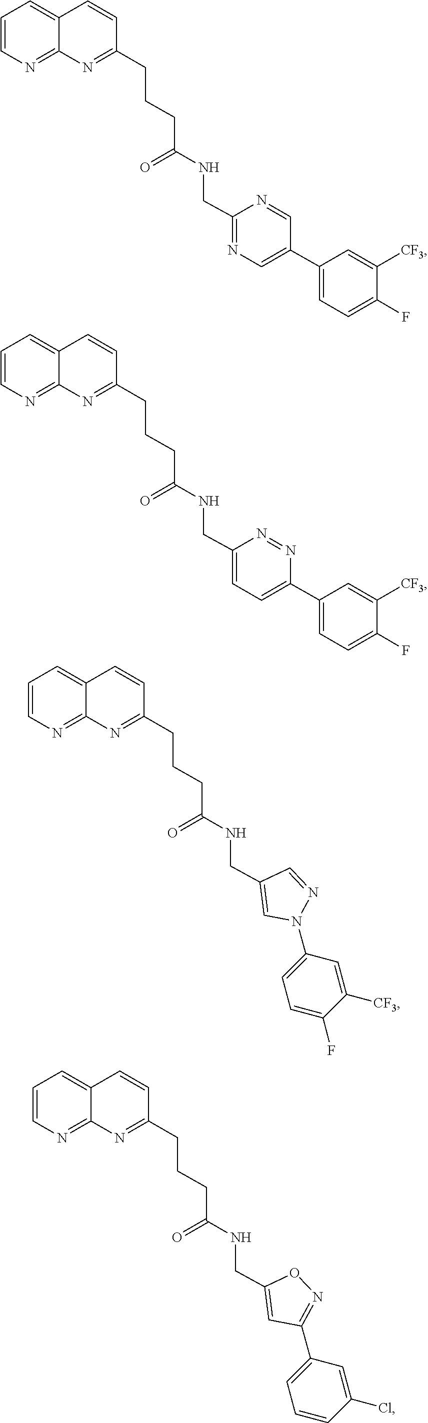 [SMILES: O=C(CCCc1ccc2cccnc2n1)NCc1ncc(-c2ccc(F)c(C(F)(F)F)c2)cn1 ; O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1]

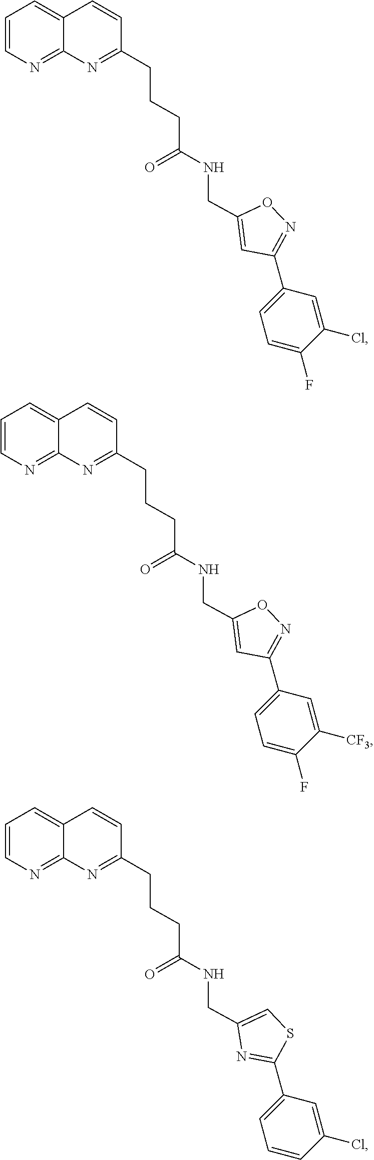 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1]

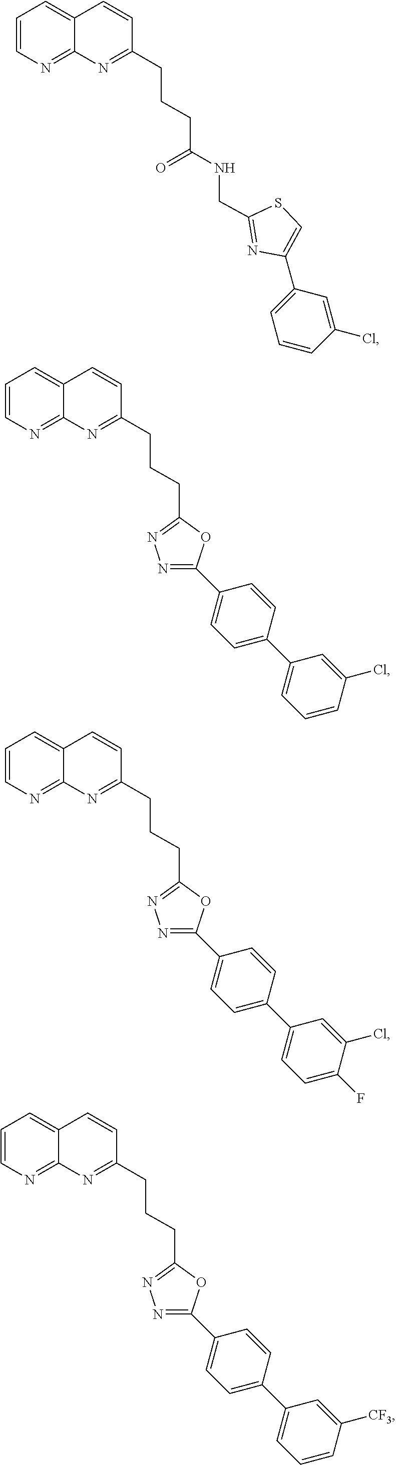 [SMILES: O=C(CCCc1ccc2cccnc2n1)NCc1nc(-c2cccc(Cl)c2)cs1 ; Clc1cccc(-c2ccc(-c3nnc(CCCc4ccc5cccnc5n4)o3)cc2)c1 ; Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1]

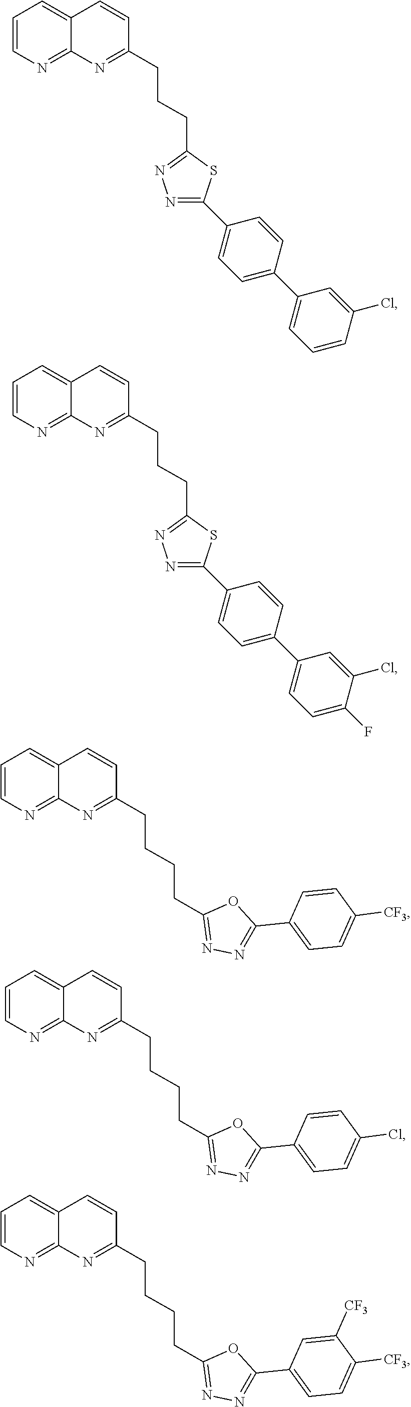 [SMILES: Clc1cccc(-c2ccc(-c3nnc(CCCc4ccc5cccnc5n4)s3)cc2)c1 ; Fc1ccc(-c2ccc(-c3nnc(CCCc4ccc5cccnc5n4)s3)cc2)cc1Cl ; FC(F)(F)c1ccc(-c2nnc(CCCCc3ccc4cccnc4n3)o2)cc1 ; Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F]

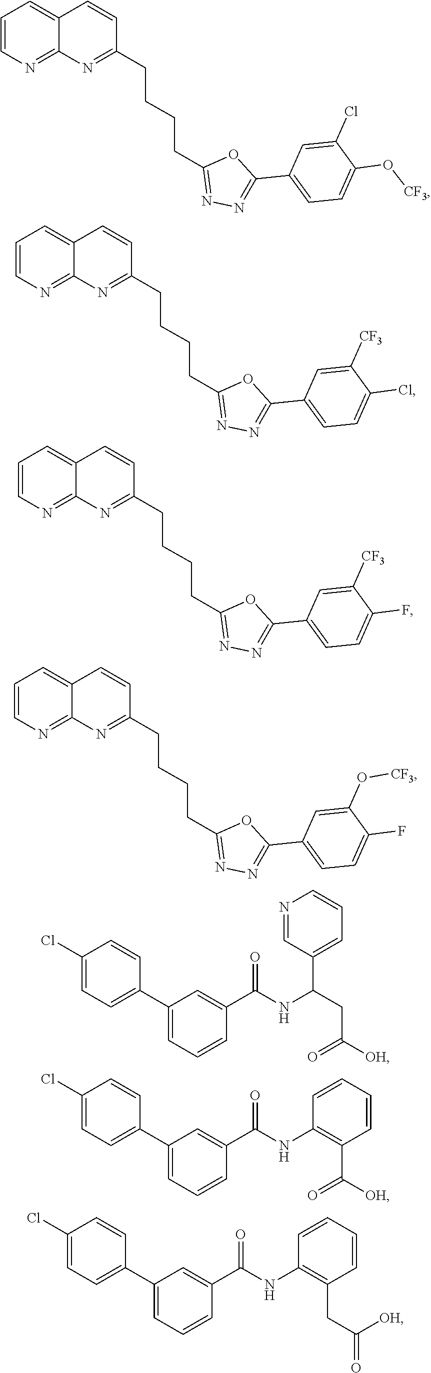

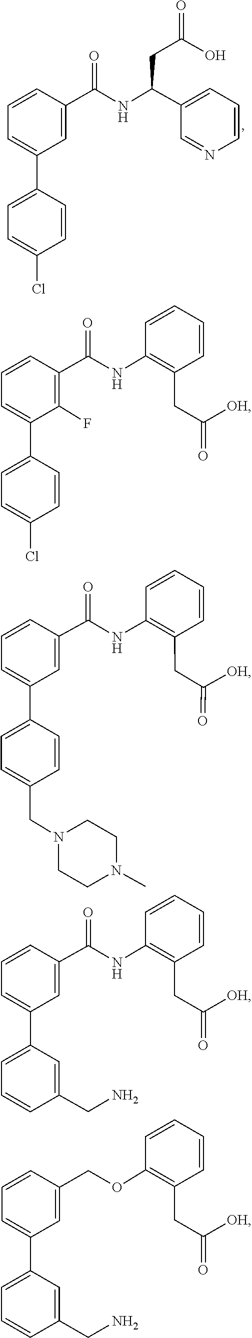

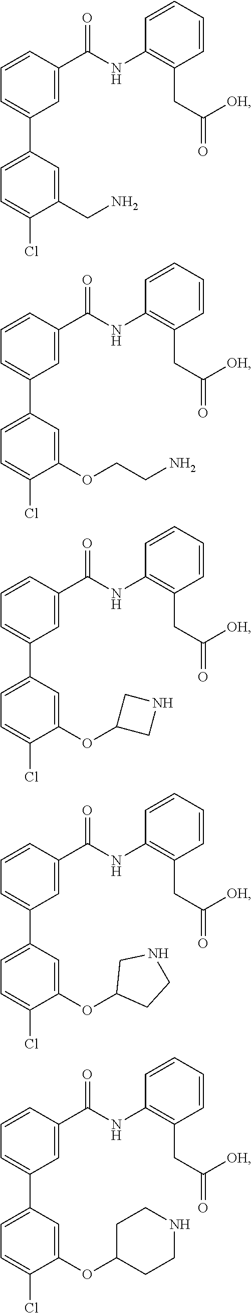 [SMILES: NCc1cc(-c2cccc(C(=O)Nc3ccccc3CC(=O)O)c2)ccc1Cl ; NCCOc1cc(-c2cccc(C(=O)Nc3ccccc3CC(=O)O)c2)ccc1Cl ; O=C(O)Cc1ccccc1NC(=O)c1cccc(-c2ccc(Cl)c(OC3CNC3)c2)c1 ; O=C(O)Cc1ccccc1NC(=O)c1cccc(-c2ccc(Cl)c(OC3CCNC3)c2)c1 ; O=C(O)Cc1ccccc1NC(=O)c1cccc(-c2ccc(Cl)c(OC3CCNCC3)c2)c1]

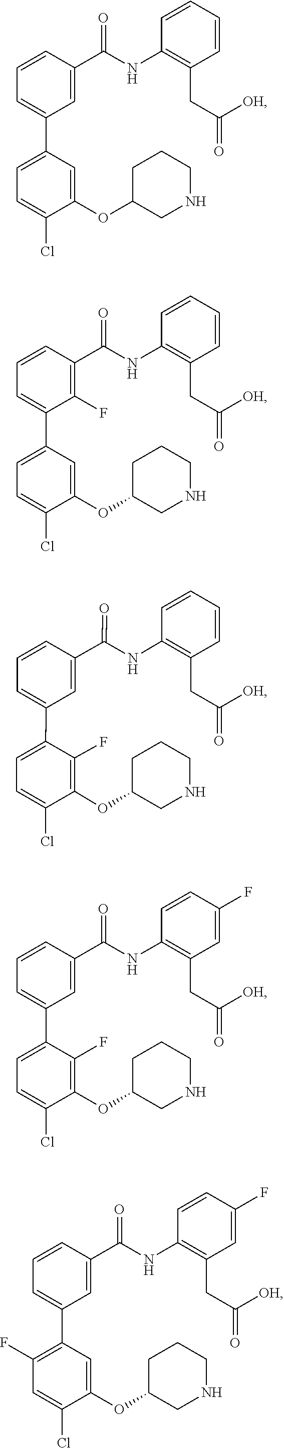 [SMILES: O=C(O)Cc1ccccc1NC(=O)c1cccc(-c2ccc(Cl)c(OC3CCCNC3)c2)c1 ; O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1]

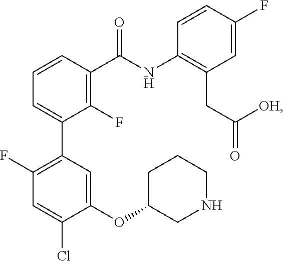 [SMILES: O=C(O)Cc1cc(F)ccc1NC(=O)c1cccc(-c2cc(O[C@@H]3CCCNC3)c(Cl)cc2F)c1F]

and combinations thereof.

In an embodiment, the inhibitor of the succinate/SUCNR1 signaling is provided as a topical formulation for use in the oral cavity. For example, 7a and/or 7b can be provided in the form of gel and applied topically onto the subgingival area by brushing daily (for gel formulation). Treatment can be carried out for the any length of time as needed, such as over a period of days, weeks or months or longer and can be done as frequently as desired. For example, it can be done daily, or weekly or more or less frequently. In various other embodiments, succinate/SUCNR1 inhibitors other than 7a and/or 7b may be used in the topical formulation.

In an embodiment, the dosage of a compound of the present disclosure can be from 1.0 mg/kg to 500 mg/kg. For example, the dosage can be used from 10 mg/kg to 100 mg/kg. For example, the dosage can be 10, 20, 30, 40, 50, 60, 70, 80, 90 and 100 mg/kg. In an embodiment, the dose is 50 mg/kg per application.

A method of treating neuroinflammation in an individual (human or non-human animal) comprising administering to the individual in need of treatment an oral gel of the present disclosure into the oral cavity of said individual, wherein the oral gel comprises an inhibitor of the succinate/succinate receptor 1 signaling pathway (such as compounds 7a and/or 7b) in the oral cavity, and a polymer (such as the polymers disclosed herein). In other embodiments, the method comprises systemically administering to the individual in need of treatment a composition comprising an inhibitor of the succinate/succinate receptor 1 signaling pathway.

The following Statements provide various embodiments of the present disclosure:
Statement 1. An oral gel comprising an inhibitor of the succinate/succinate receptor 1 signaling pathway in the oral cavity, and one or more polymers, wherein the inhibitor of succinate/succinate receptor has the following structure or a succinate salt thereof:

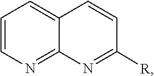

where R is selected from the group consisting of a hydrogen atom, an alkyl group, COOR′ group, and

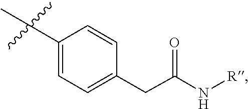

where R′ is an alkyl group and R″ is selected from the group consisting of a hydrogen atom, an alkyl group, and

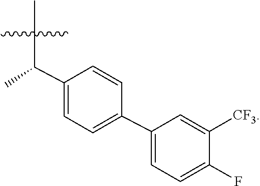 [SMILES: *[C@@H](C)c1ccc(-c2ccc(F)c(C(F)(F)F)c2)cc1]

Statement 2. An oral gel of Statement 1, wherein the succinate/succinate receptor 1 inhibitor has the following structure:

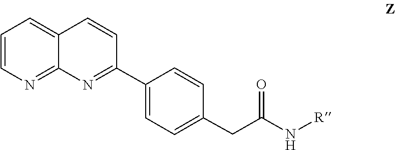

where R″ is selected from the group consisting of a hydrogen atom, an alkyl group, and

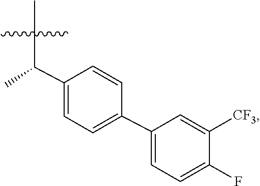 [SMILES: *[C@@H](C)c1ccc(-c2ccc(F)c(C(F)(F)F)c2)cc1]

and where Z is optional and when present, is a succinate group.
Statement 3. An oral gel of Statement 1, wherein the succinate/succinate receptor 1 inhibitor is selected from the group consisting of

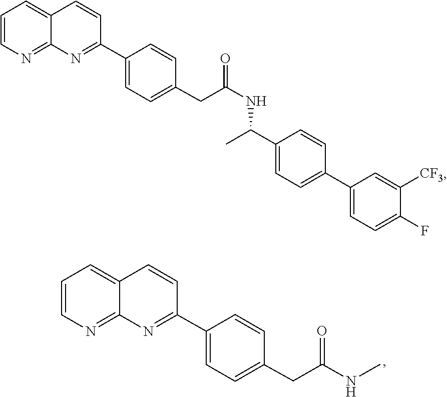 [SMILES: C[C@H](NC(=O)Cc1ccc(-c2ccc3cccnc3n2)cc1)c1ccc(-c2ccc(F)c(C(F)(F)F)c2)cc1 ; CNC(=O)Cc1ccc(-c2ccc3cccnc3n2)cc1]

and succinate salts thereof.
Statement 4. An oral gel of Statement 3, wherein the succinate/succinate receptor 1 inhibitor is

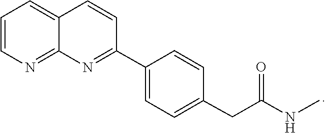 [SMILES: CNC(=O)Cc1ccc(-c2ccc3cccnc3n2)cc1]

Statement 5. An oral gel of Statement 4, wherein the concentration of

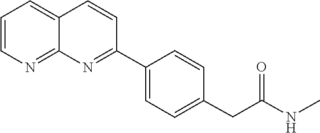 [SMILES: CNC(=O)Cc1ccc(-c2ccc3cccnc3n2)cc1]

is 1-10 mg/mL.
Statement 6. An oral gel of Statement 5, wherein the concentration is 3-7 mg/mL.
Statement 7. An oral gel of Statements 5 or 6, wherein the concentration is 4-6 mg/mL.
Statement 8. An oral gel of any one of Statements 5-7, wherein the concentration is around 5 mg/mL.
Statement 9. An oral gel of any one of Statements 5-8, wherein the concentration is 5 mg/mL.
Statement 10. An oral gel of any one of the preceding Statements, wherein the one or more polymers are chosen from poly lactic glycolic acid (PLGA), polycaprolactone (PCL) (e.g., poly-ε-caprolactone ester terminated), poly(D,L-lactide-co-glycolide) (which may be ester terminated), poly(D,L-lactide) (which may be ester-terminated), chitosan, starch, polylactic acid, alginate, and the like, and combinations thereof.
Statement 11. An oral gel of Statement 10, wherein the one or more polymers are PLGA, poly-ε-caprolactone ester terminated, poly(D,L-lactide) ester-terminated, and poly(D,L-lactide-co-glycolide) ester-terminated.
Statement 12. An oral gel of any one of the preceding Statements, wherein the oral gel further comprises a solvent.
Statement 13. An oral gel of Statement 12, wherein the solvent is an organic solvent.
Statement 14. An oral gel of Statements 12 or 13, wherein the solvent is benzyl benzoate, N-methyl-2-pyrrolidinone, or a combination thereof.
Statement 15. An oral gel of any one of the preceding Statements, wherein the succinate/succinate receptor 1 inhibitor is

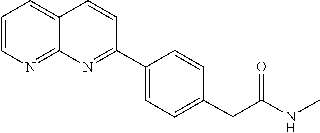 [SMILES: CNC(=O)Cc1ccc(-c2ccc3cccnc3n2)cc1]

(e.g., the succinate/succinate receptor 1 inhibitor having a concentration of about 5.0 mg/mL (e.g., 5.0 mg/mL)), the polymer is PLGA (e.g., PLGA having a concentration of about 10% w/v (e.g., 10% w/v)), and the solvent is a 30% solution (by volume) of benzyl benzoate in N-methyl-2-pyrrolidinone.
Statement 16. A method of treating neuroinflammation in an individual in need thereof, the method comprising administering to the oral cavity of the individual the oral gel of any one of the preceding Statements.
Statement 17. An method of Statement 16, wherein the succinate/succinate receptor 1 inhibitor in the oral gel has the following structure:

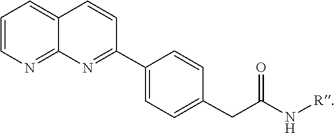

Statement 18. An method of Statement 17, wherein the succinate/succinate receptor 1 inhibitor in the oral gel is selected from the group consisting of:

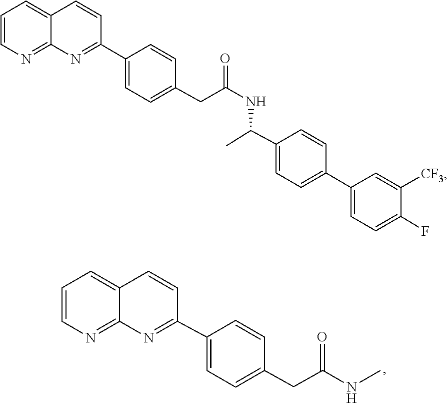 [SMILES: C[C@H](NC(=O)Cc1ccc(-c2ccc3cccnc3n2)cc1)c1ccc(-c2ccc(F)c(C(F)(F)F)c2)cc1 ; CNC(=O)Cc1ccc(-c2ccc3cccnc3n2)cc1]

succinate salts thereof, and combinations thereof.
Statement 19. An method of any one of Statements 16-18, wherein the individual is afflicted with periodontal bone loss.
Statement 20. An method any one of Statements 16-19, wherein the individual is a human.
Statement 21. An method of any one of Statements 16-19, wherein the individual is a non-human animal.
Statement 22. An method of Statement 21, wherein the non-human animal is a cat or a dog.
Statement 23. An method of Statement 21, wherein the non-human animal is an agricultural animal.
Statement 24. An method of any one of Statements 16-23, wherein the succinate/succinate receptor 1 inhibitor is

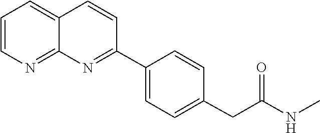 [SMILES: CNC(=O)Cc1ccc(-c2ccc3cccnc3n2)cc1]

(e.g., the succinate/succinate receptor 1 inhibitor having a concentration of about 5.0 mg/mL (e.g., 5.0 mg/mL)), the polymer is PLGA (e.g., PLGA having a concentration of about 10% w/v (e.g., 10% w/v)), and the solvent is a 30% solution (by volume) of benzyl benzoate in N-methyl-2-pyrrolidinone.
Statement 25. A method for treating neuroinflammation in an individual, comprising: administering to the individual a composition comprising a succinate/succinate receptor 1 inhibitor, wherein the neuroinflammation in the individual is ameliorated or reduced, with that proviso that when the succinate/succinate receptor 1 inhibitor has the following structure:

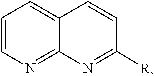

where R is selected from the group consisting of a hydrogen atom, an alkyl group, COOR′ group, and

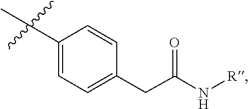

where R′ is an alkyl group and R″ is selected from the group consisting of a hydrogen atom, an alkyl group, and

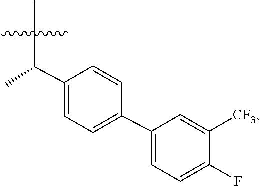 [SMILES: *[C@@H](C)c1ccc(-c2ccc(F)c(C(F)(F)F)c2)cc1]

the composition is not a thin film having a thickness of from 0.05 mm to 0.4 mm. The administration may be a location other than the brain of the individual. In various other embodiments, the administration is directly into the brain.
Statement 26. A method of Statement 25, wherein the neuroinflammation is associated with a disease, a disorder, an infection, an injury, an immune response, or aging.
Statement 27. A method of claim 26, wherein the infection is caused by a virus, bacterium, fungus, protozoa, or parasite.
Statement 28. A method according to Statement 26 or Statement 27, wherein the infection results in encephalitis or meningitis.
Statement 29. A method according to Statement 26, wherein the disease is chosen from acute disseminated encephalomyelitis, acute optic neuritis, transverse myelitis, and neuromyelitis optica.
Statement 30. A method of Statement 26, wherein the disease is a neurodegenerative disease.
Statement 31. A method of Statement 30, wherein neurodegenerative disease is chosen from amyotrophic lateral sclerosis (ALS), Alzheimer's Disease, Parkinson's Disease, and multiple sclerosis.
Statement 32. A method of Statement 26, wherein the disorder is a psychiatric disorder.
Statement 33. A method of Statement 32, wherein the psychiatric disorder is schizophrenia, autism, depression, or a mood disorder.
Statement 34. A method of Statement 25, wherein the neuroinflammation is associated with Adrenal Leukodystrophy (ALD), Alcoholism, Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia, Spinal muscular atrophy, Steele-Richardson-Olszewski disease, Tabes dorsalis, Toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, Hereditary Spastic Paraparesis, Mitochondrial myopathy, periodontal disease, Friedreich Ataxia, or the like, or combinations thereof.
Statement 35. A method according to any one of Statements 25-34, wherein the succinate/succinate receptor 1 inhibitor is chosen from:

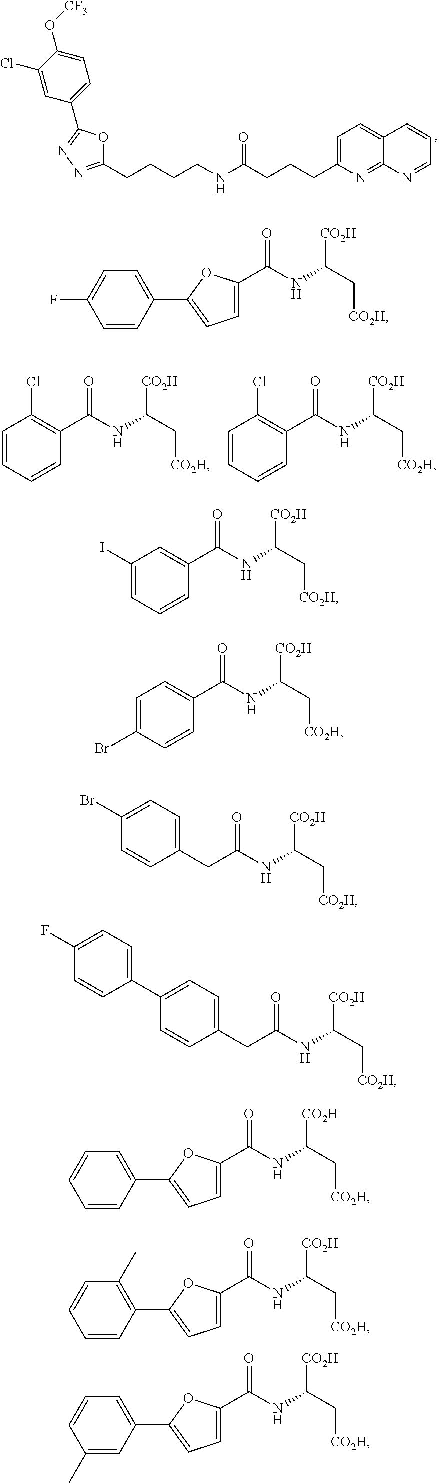

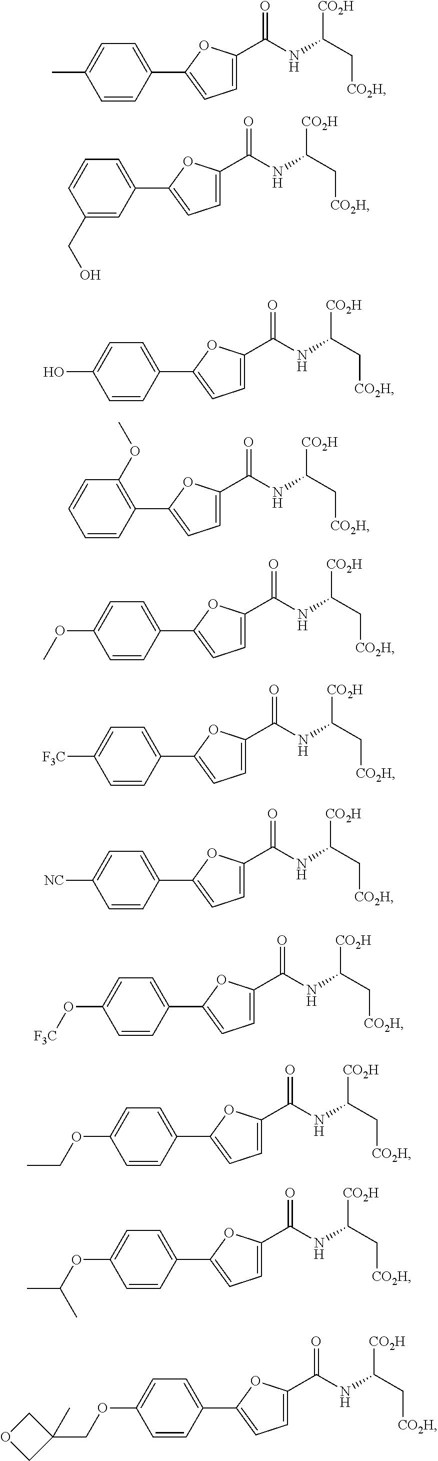

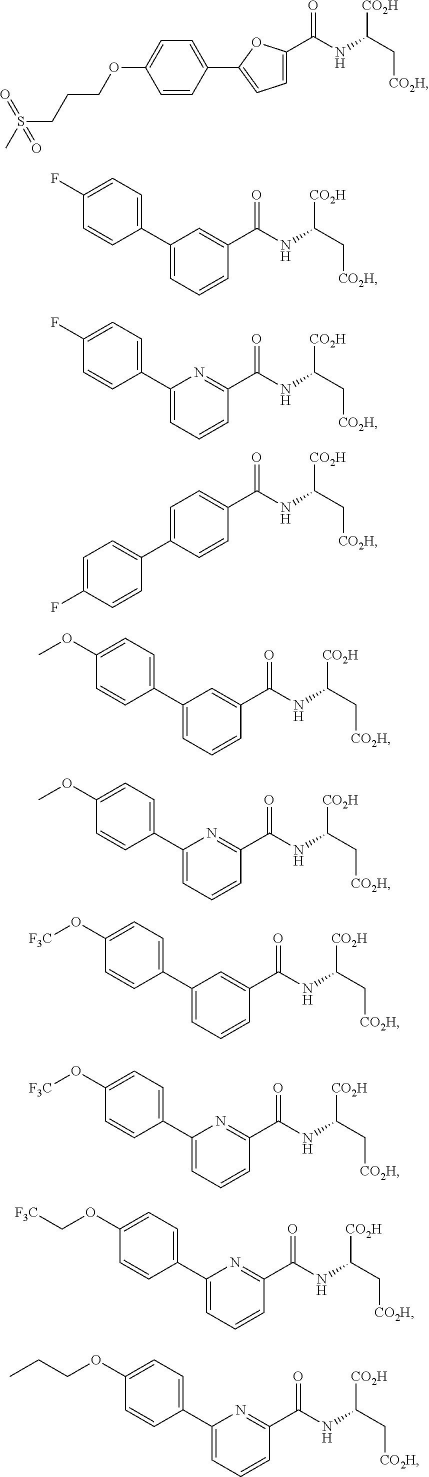

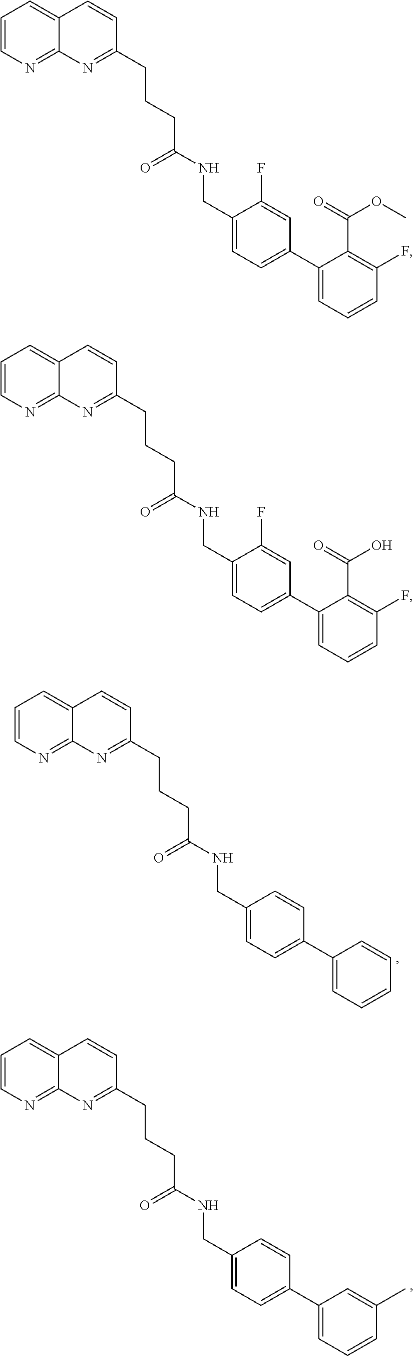 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1]

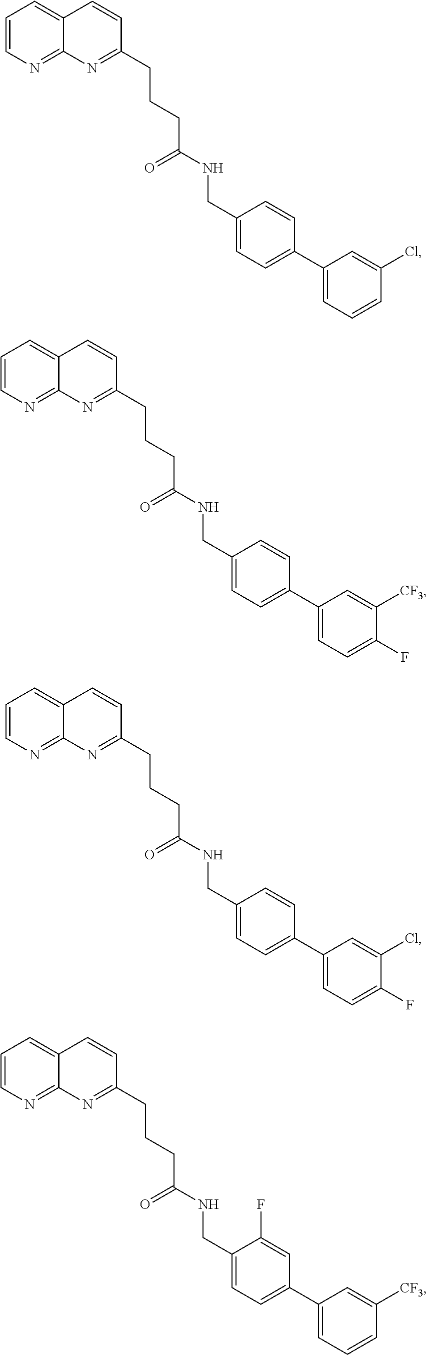 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1 ; O=C(CCCc1ccc2cccnc2n1)NCc1ccc(-c2cccc(C(F)(F)F)c2)cc1F]

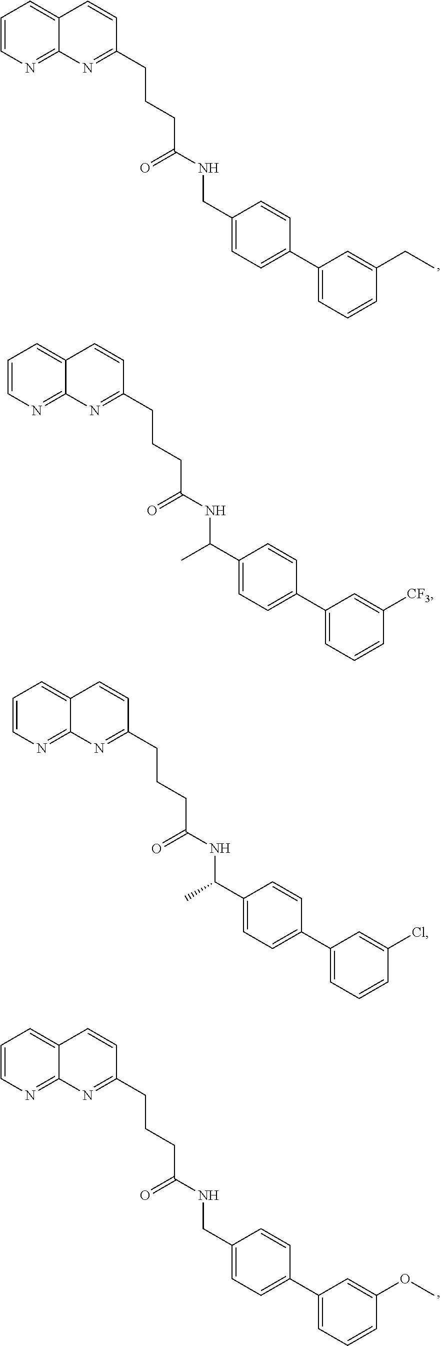 [SMILES: CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1 ; COc1cccc(-c2ccc(CNC(=O)CCCc3ccc4cccnc4n3)cc2)c1]

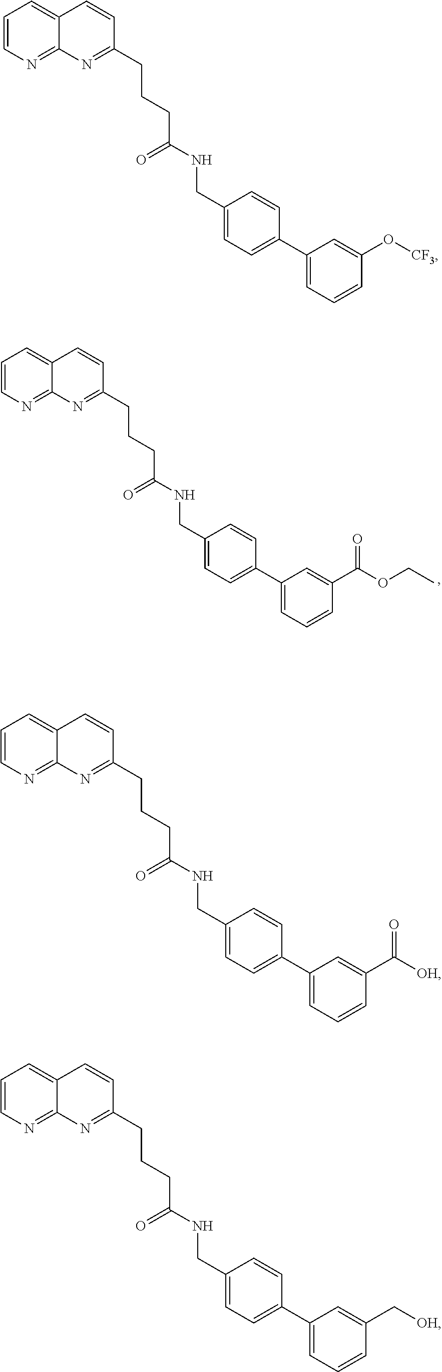 [SMILES: O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1 ; O=C(CCCc1ccc2cccnc2n1)NCc1ccc(-c2cccc(CO)c2)cc1]

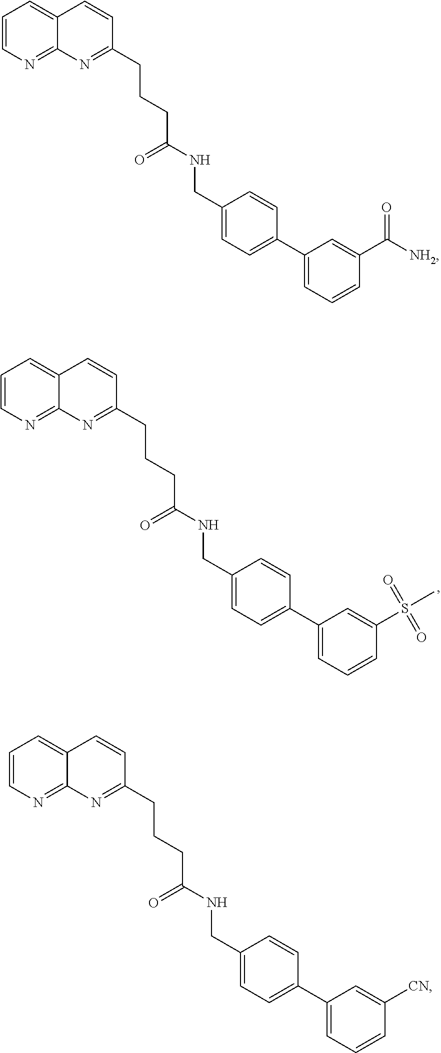 [SMILES: NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1]

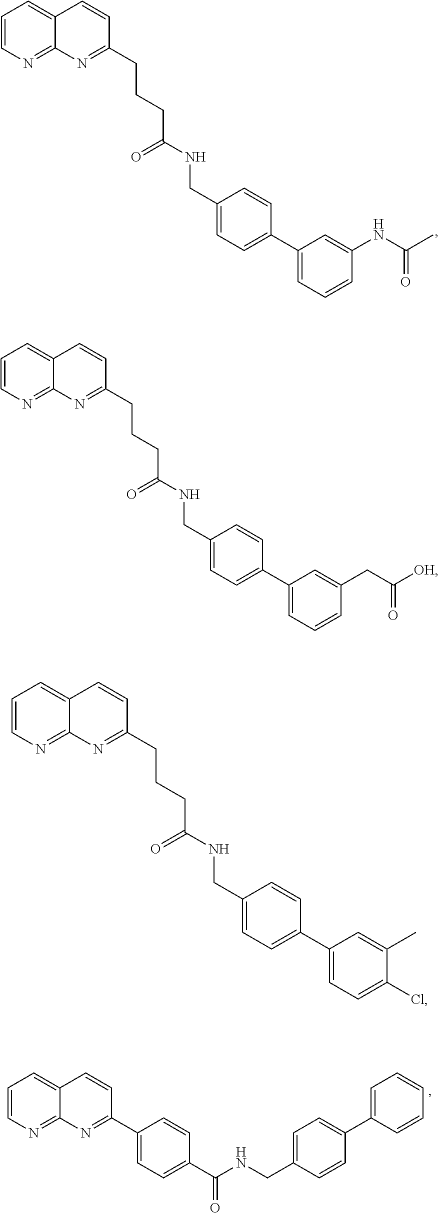 [SMILES: CC(=O)Nc1cccc(-c2ccc(CNC(=O)CCCc3ccc4cccnc4n3)cc2)c1 ; O=C(O)Cc1cccc(-c2ccc(CNC(=O)CCCc3ccc4cccnc4n3)cc2)c1 ; Cc1cc(-c2ccc(CNC(=O)CCCc3ccc4cccnc4n3)cc2)ccc1Cl ; O=C(NCc1ccc(-c2ccccc2)cc1)c1ccc(-c2ccc3cccnc3n2)cc1]

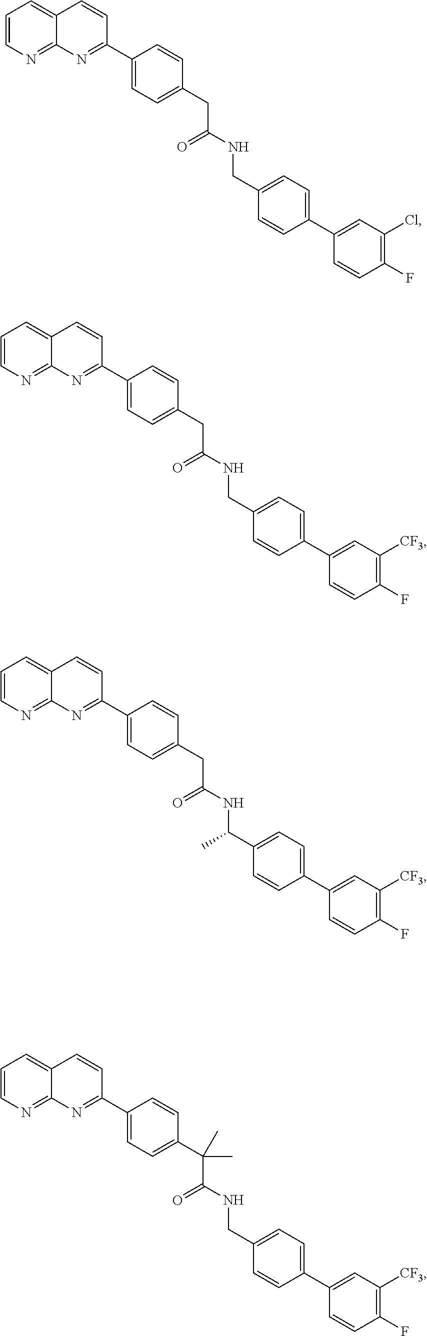 [SMILES: O=C(Cc1ccc(-c2ccc3cccnc3n2)cc1)NCc1ccc(-c2ccc(F)c(Cl)c2)cc1 ; O=C(Cc1ccc(-c2ccc3cccnc3n2)cc1)NCc1ccc(-c2ccc(F)c(C(F)(F)F)c2)cc1 ; C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1]

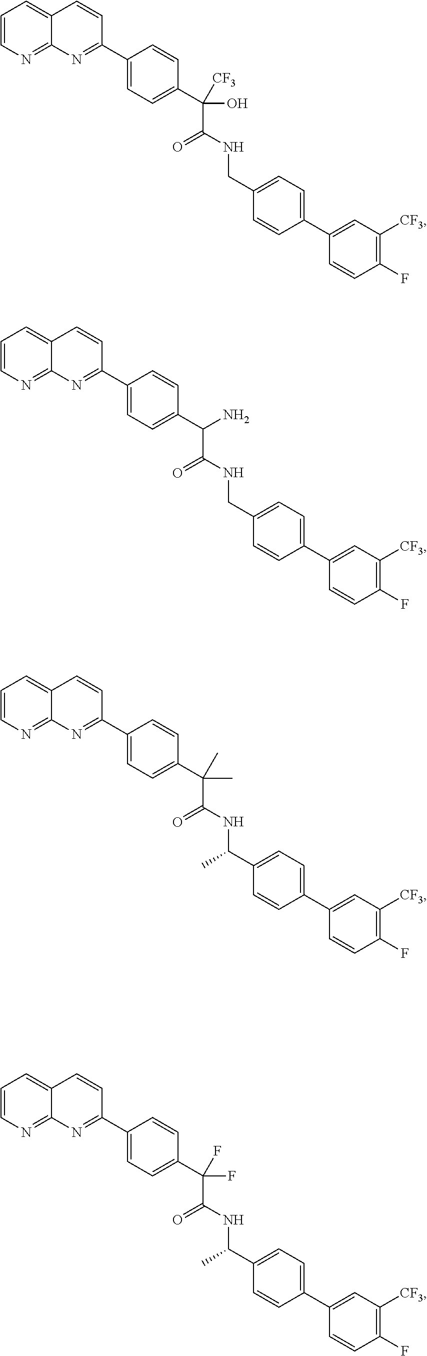 [SMILES: O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1 ; C[C@H](NC(=O)C(F)(F)c1ccc(-c2ccc3cccnc3n2)cc1)c1ccc(-c2ccc(F)c(C(F)(F)F)c2)cc1]

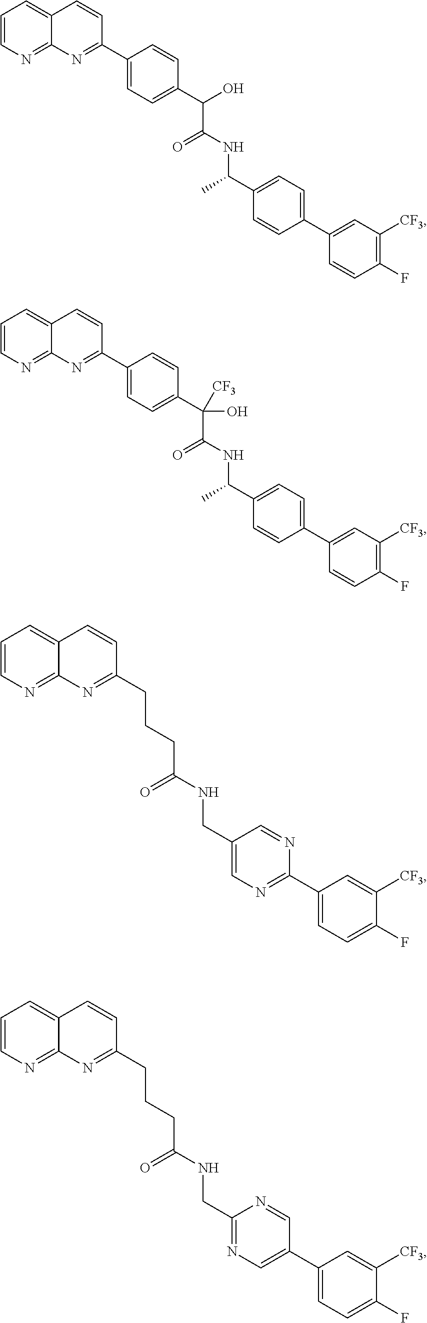 [SMILES: C[C@H](NC(=O)C(O)c1ccc(-c2ccc3cccnc3n2)cc1)c1ccc(-c2ccc(F)c(C(F)(F)F)c2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1 ; O=C(CCCc1ccc2cccnc2n1)NCc1ncc(-c2ccc(F)c(C(F)(F)F)c2)cn1]

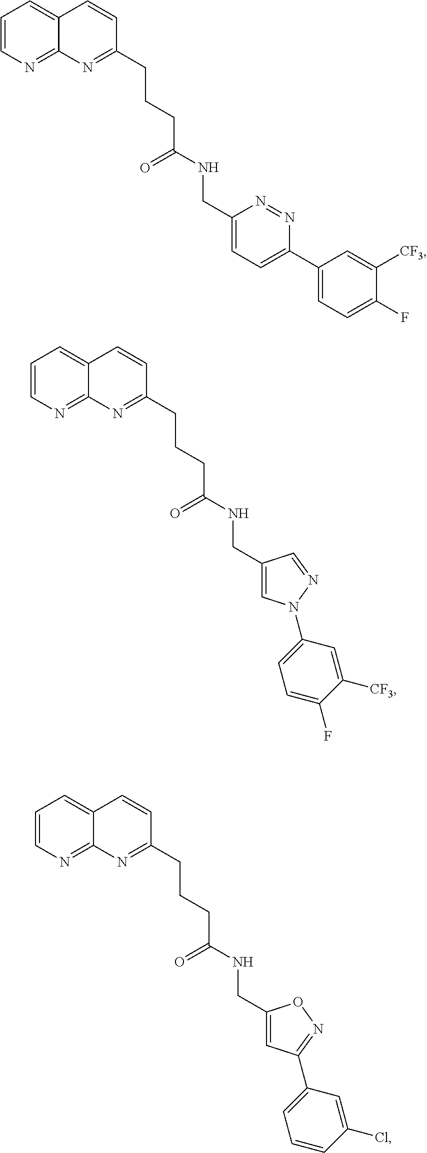 [SMILES: O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1]

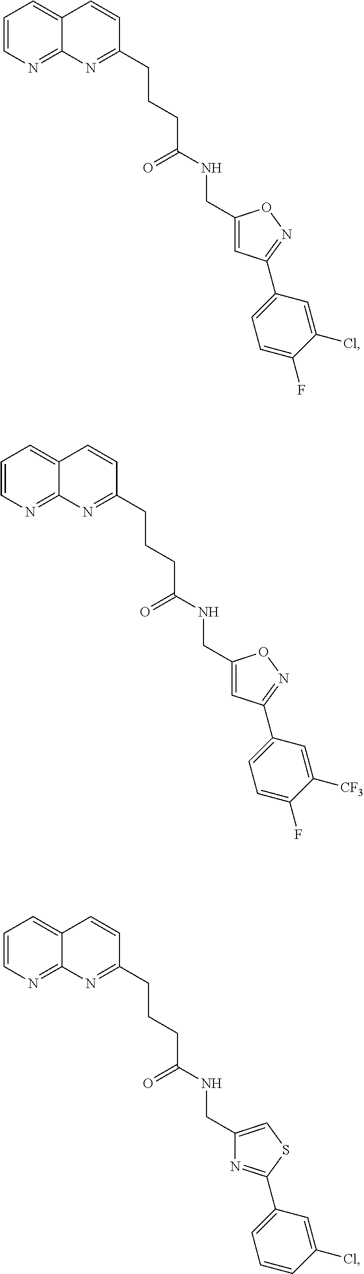 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1]

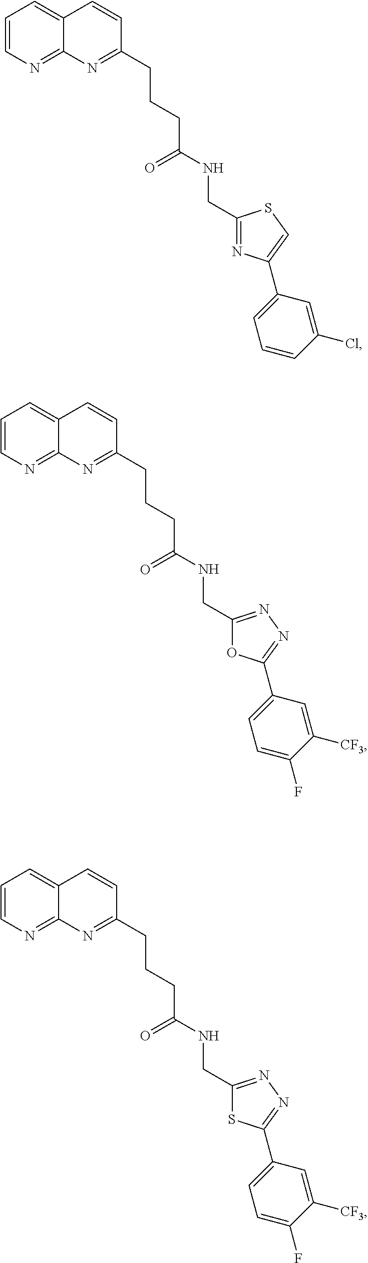 [SMILES: O=C(CCCc1ccc2cccnc2n1)NCc1nc(-c2cccc(Cl)c2)cs1 ; O=C(CCCc1ccc2cccnc2n1)NCc1nnc(-c2ccc(F)c(C(F)(F)F)c2)o1 ; O=C(CCCc1ccc2cccnc2n1)NCc1nnc(-c2ccc(F)c(C(F)(F)F)c2)s1]

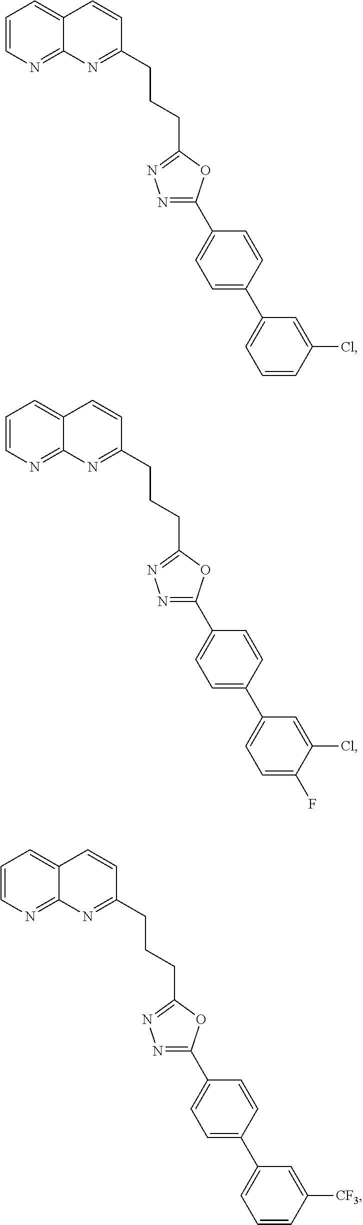 [SMILES: Clc1cccc(-c2ccc(-c3nnc(CCCc4ccc5cccnc5n4)o3)cc2)c1 ; Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1]

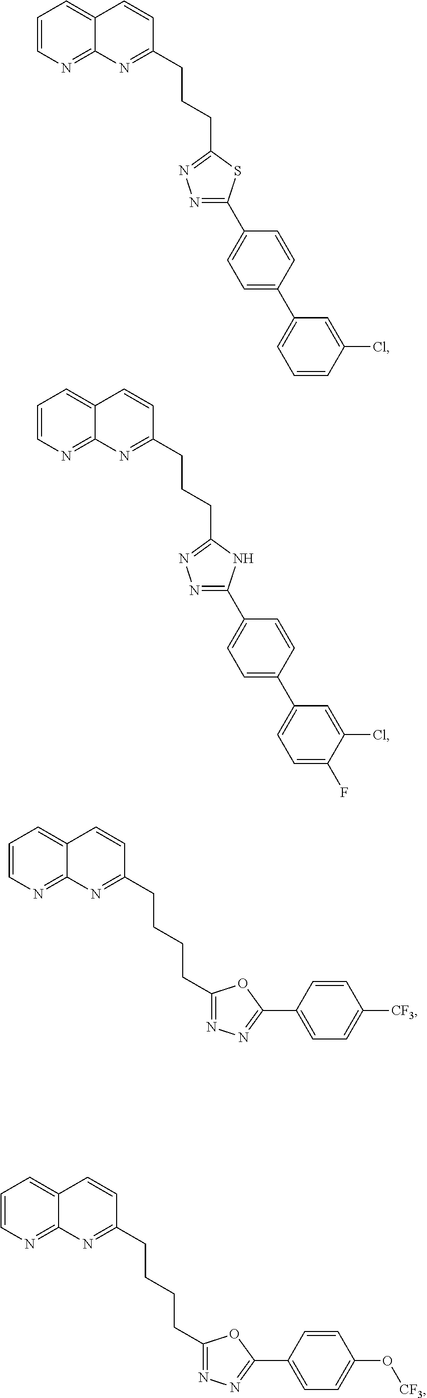 [SMILES: Clc1cccc(-c2ccc(-c3nnc(CCCc4ccc5cccnc5n4)s3)cc2)c1 ; Fc1ccc(-c2ccc(-c3nnc(CCCc4ccc5cccnc5n4)[nH]3)cc2)cc1Cl ; FC(F)(F)c1ccc(-c2nnc(CCCCc3ccc4cccnc4n3)o2)cc1 ; FC(F)(F)Oc1ccc(-c2nnc(CCCCc3ccc4cccnc4n3)o2)cc1]

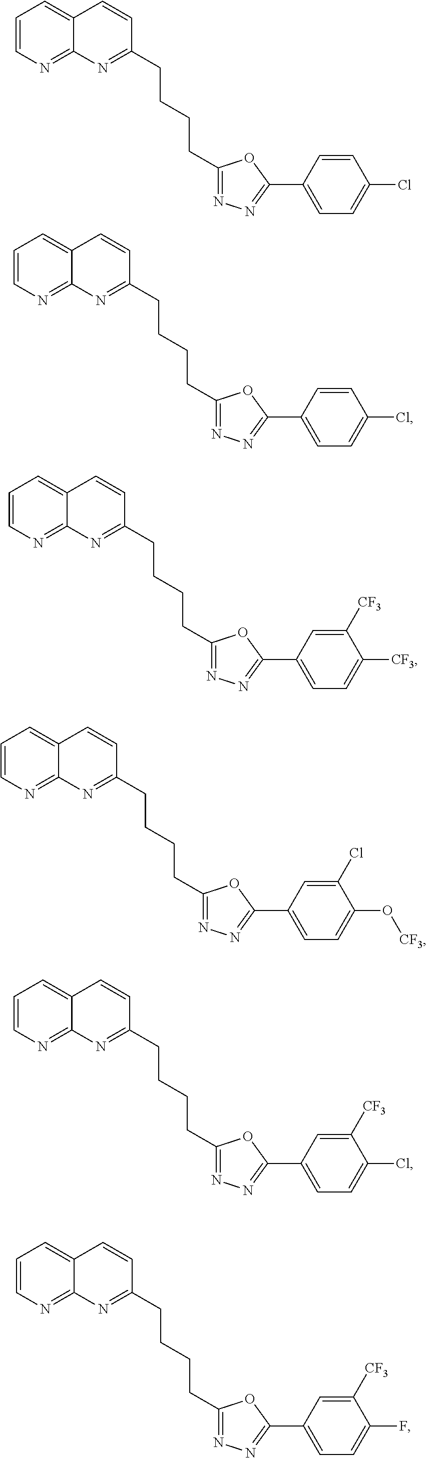 [SMILES: Clc1ccc(-c2nnc(CCCCc3ccc4cccnc4n3)o2)cc1 ; Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F ; FC(F)(F)Oc1ccc(-c2nnc(CCCCc3ccc4cccnc4n3)o2)cc1Cl ; FC(F)(F)c1cc(-c2nnc(CCCCc3ccc4cccnc4n3)o2)ccc1Cl ; Fc1ccc(-c2nnc(CCCCc3ccc4cccnc4n3)o2)cc1C(F)(F)F]

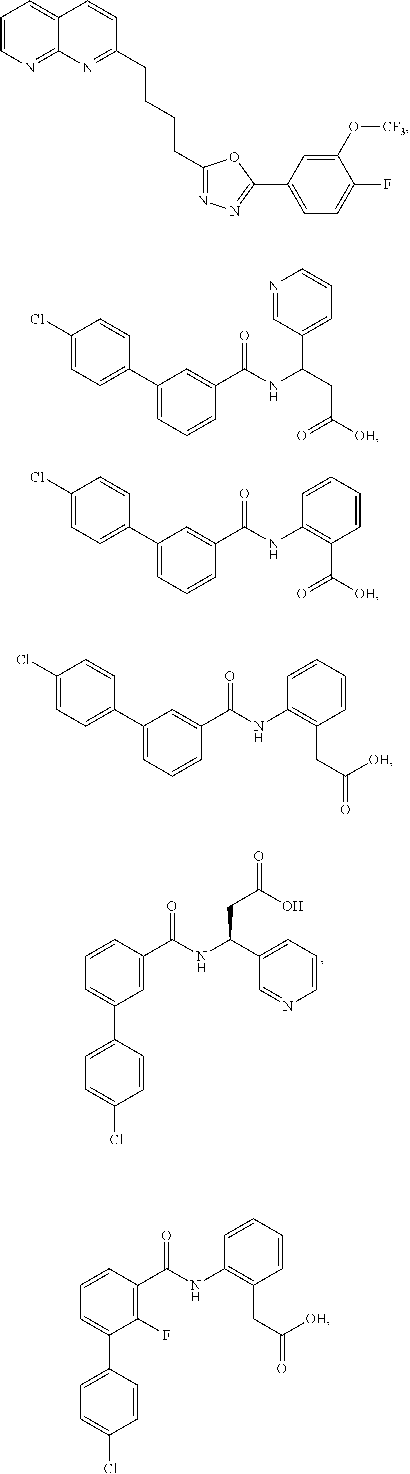 [SMILES: Fc1ccc(-c2nnc(CCCCc3ccc4cccnc4n3)o2)cc1OC(F)(F)F ; O=C(O)CC(NC(=O)c1cccc(-c2ccc(Cl)cc2)c1)c1cccnc1 ; O=C(Nc1ccccc1C(=O)O)c1cccc(-c2ccc(Cl)cc2)c1 ; O=C(O)Cc1ccccc1NC(=O)c1cccc(-c2ccc(Cl)cc2)c1 ; O=C(O)C[C@H](NC(=O)c1cccc(-c2ccc(Cl)cc2)c1)c1cccnc1 ; O=C(O)Cc1ccccc1NC(=O)c1cccc(-c2ccc(Cl)cc2)c1F]

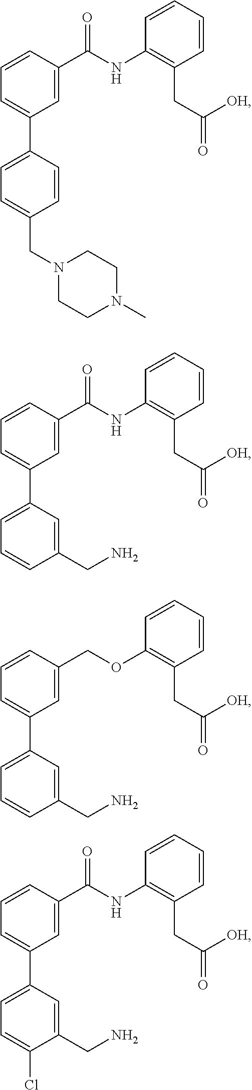

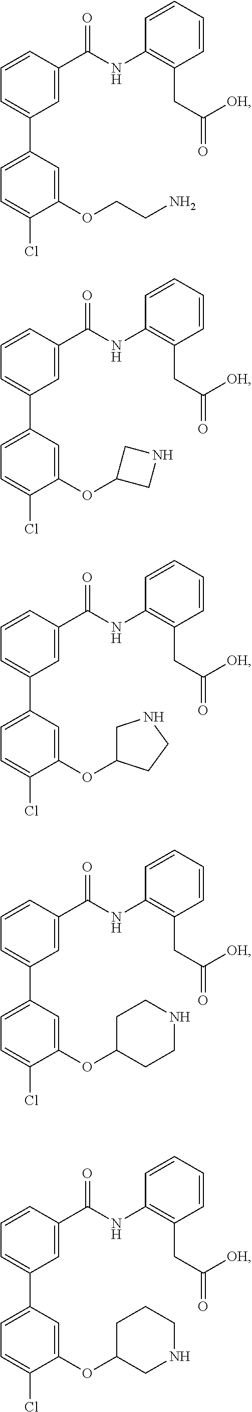 [SMILES: NCCOc1cc(-c2cccc(C(=O)Nc3ccccc3CC(=O)O)c2)ccc1Cl ; O=C(O)Cc1ccccc1NC(=O)c1cccc(-c2ccc(Cl)c(OC3CNC3)c2)c1 ; O=C(O)Cc1ccccc1NC(=O)c1cccc(-c2ccc(Cl)c(OC3CCNC3)c2)c1 ; O=C(O)Cc1ccccc1NC(=O)c1cccc(-c2ccc(Cl)c(OC3CCNCC3)c2)c1 ; O=C(O)Cc1ccccc1NC(=O)c1cccc(-c2ccc(Cl)c(OC3CCCNC3)c2)c1]

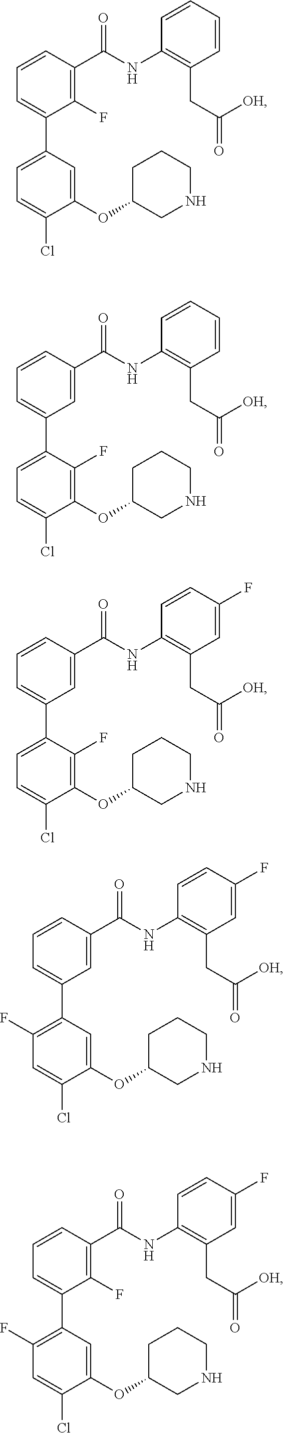 [SMILES: O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1 ; O=C(O)Cc1cc(F)ccc1NC(=O)c1cccc(-c2cc(O[C@@H]3CCCNC3)c(Cl)cc2F)c1F]

and combinations thereof.
Statement 36. A method according to any one of Statements 25-35, wherein the succinate/succinate receptor 1 inhibitor has the following structure or a succinate salt thereof:

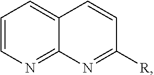

where R is selected from the group consisting of a hydrogen atom, an alkyl group, COOR′ group, and

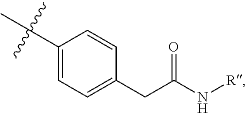

where R′ is an alkyl group and R″ is selected from the group consisting of a hydrogen atom, an alkyl group, and

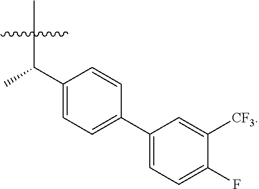 [SMILES: *[C@@H](C)c1ccc(-c2ccc(F)c(C(F)(F)F)c2)cc1]

Statement 37. A method of Statement 36, wherein the succinate/succinate receptor 1 inhibitor has the following structure:

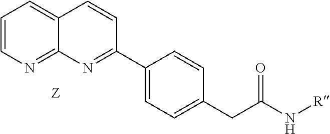

where R″ is selected from the group consisting of a hydrogen atom, an alkyl group, and

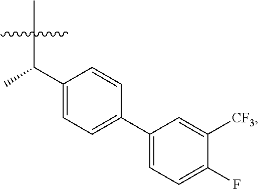 [SMILES: *[C@@H](C)c1ccc(-c2ccc(F)c(C(F)(F)F)c2)cc1]

and where Z is optional and when present, is a succinate group.
Statement 38. A method of Statement 36 or Statement 37, wherein the succinate/succinate receptor 1 inhibitor is selected from the group consisting of

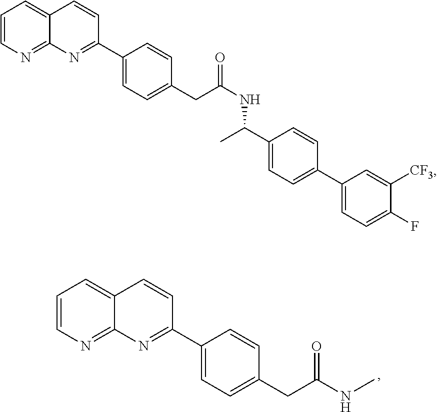 [SMILES: C[C@H](NC(=O)Cc1ccc(-c2ccc3cccnc3n2)cc1)c1ccc(-c2ccc(F)c(C(F)(F)F)c2)cc1 ; CNC(=O)Cc1ccc(-c2ccc3cccnc3n2)cc1]

and succinate salts thereof.
Statement 39. A method according to any one of Statements 36-38, wherein the succinate/succinate receptor 1 inhibitor is

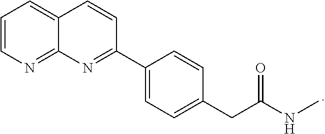 [SMILES: CNC(=O)Cc1ccc(-c2ccc3cccnc3n2)cc1]

Statement 40. A method of Statement 39, wherein

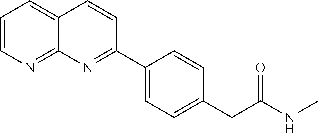 [SMILES: CNC(=O)Cc1ccc(-c2ccc3cccnc3n2)cc1]

is administered at a concentration of 1-10 mg/mL.
Statement 41. A method of Statement 40, wherein the concentration is 3-7 mg/mL.
Statement 42. A method of Statement 41, wherein the concentration is 4-6 mg/mL.
Statement 43. A method of Statement 42, wherein the concentration is around or about 5 mg/mL or is 5 mg/mL.
Statement 44. A method according to any one of Statements 25-43, wherein the composition is a film, a gel, a capsule, a tablet, a plurality of nanoparticles, or liquid composition.
Statement 45. A method according to any one of Statements 25-44, wherein the composition further comprises a pharmaceutically acceptable carrier.
Statement 46. A method according to any one of Statements 25-45, wherein the composition is a gel comprising one or more polymers.
Statement 47. A method of Statement 46, wherein the one or more polymers are chosen from poly lactic glycolic acid (PLGA), polycaprolactone (PCL), poly-ε-caprolactone ester terminated, poly(D,L-lactide-co-glycolide, poly(D,L-lactide-co-glycolide ester terminated, poly(D,L-lactide, poly(D,L-lactide) ester terminated, chitosan, starch, polylactic acid, alginate, and combinations thereof.
Statement 48. A method of Statement 47, wherein the one or more polymers are chosen from PLGA, poly-ε-caprolactone ester terminated, poly(D,L-lactide) ester-terminated, poly(D,L-lactide-co-glycolide) ester terminated, and combinations thereof.
Statement 49. A method according to any one of Statements 46-48, wherein the gel further comprises one or more solvent.
Statement 50. A method of Statement 49, wherein the solvent is benzyl benzoate, N-methyl-2-pyrrolidinone, or a combination thereof.
Statement 51. A method according to any one of Statements 25-50, wherein the composition is a gel and the succinate/succinate receptor 1 inhibitor is

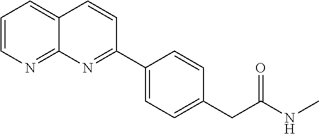 [SMILES: CNC(=O)Cc1ccc(-c2ccc3cccnc3n2)cc1]

the polymer is PLGA, and the oral gel further comprises a 30% solution of benzyl benzoate in N-methyl-2-pyrrolidinone.
Statement 52. A method of Statement 51, wherein the succinate/succinate receptor 1 inhibitor is

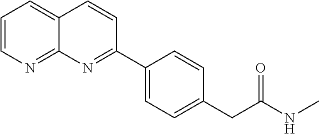 [SMILES: CNC(=O)Cc1ccc(-c2ccc3cccnc3n2)cc1]

having a concentration of 5.0 mg/mL, the polymer is PLGA having a concentration of 10% w/v, and the solvent is a 30% solution of benzyl benzoate in N-methyl-2-pyrrolidinone.
Statement 53. A method according to any one of Statements 25-52, wherein the composition is topically applied in the oral cavity of the individual.
Statement 54. A method according to any one of Statements 25-45, wherein the composition is administered systemically.
Statement 55. A method for reducing the expression of pro-inflammatory cytokines, comprising administering to the individual at a location other than the brain of the individual a composition comprising a succinate/succinate receptor 1 inhibitor, wherein the expression of one or more pro-inflammatory cytokines is reduced, with that proviso that when the succinate/succinate receptor 1 inhibitor has the following structure:

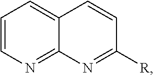

where R is selected from the group consisting of a hydrogen atom, an alkyl group, COOR′ group, and

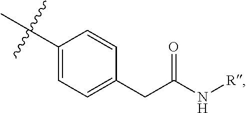

where R′ is an alkyl group and R″ is selected from the group consisting of a hydrogen atom, an alkyl group, and

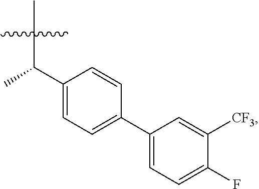 [SMILES: *[C@@H](C)c1ccc(-c2ccc(F)c(C(F)(F)F)c2)cc1]

the composition is not a thin film having a thickness of from 0.05 mm to 0.4 mm. The administration may be a location other than the brain of the individual. In various other embodiments, the administration is directly into the brain.
Statement 56. A method of Statement 55, wherein the one or more pro-inflammatory cytokines are TNFα, IL113, or a combination thereof.
Statement 57. A method of Statement 55 or Statement 56, wherein the succinate/succinate receptor 1 inhibitor is chosen from:

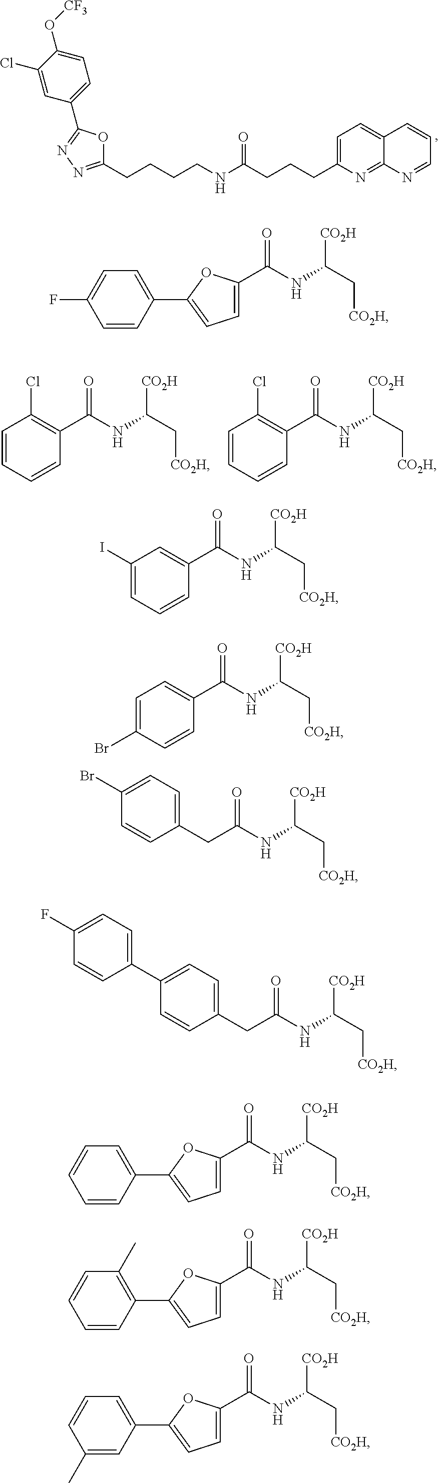

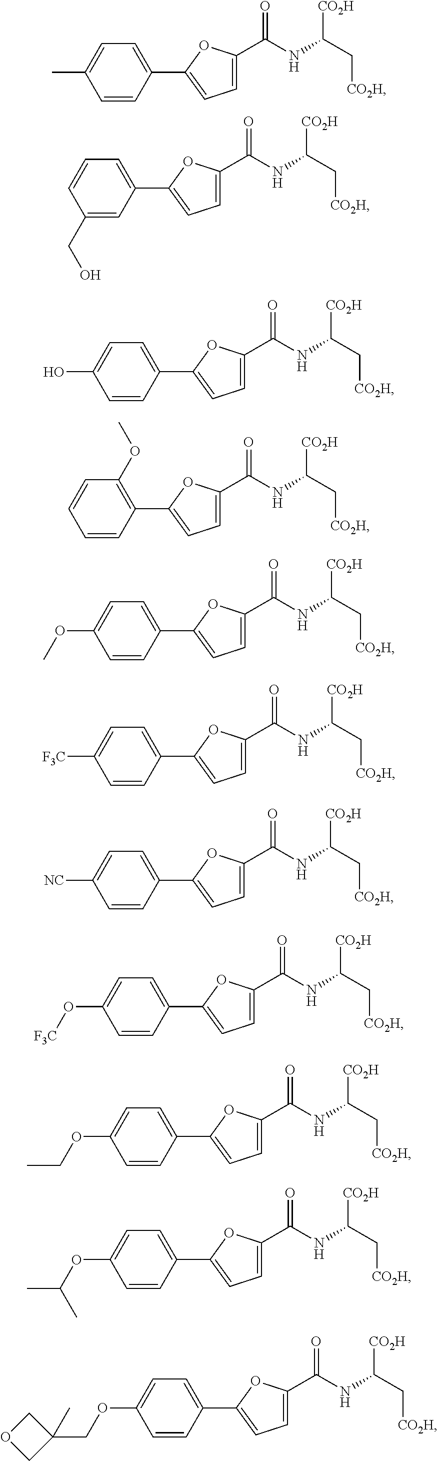

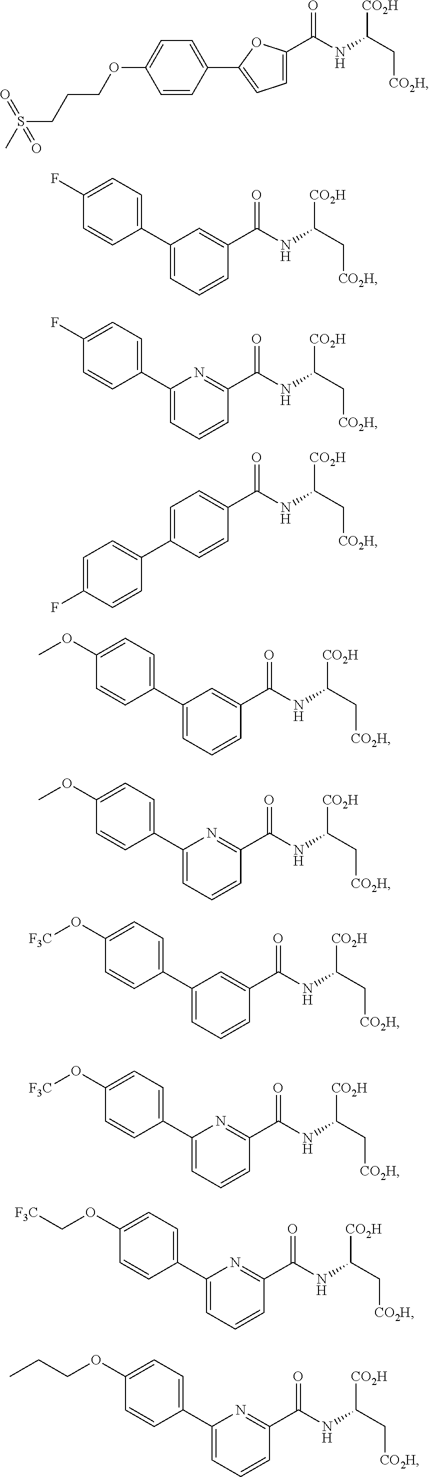

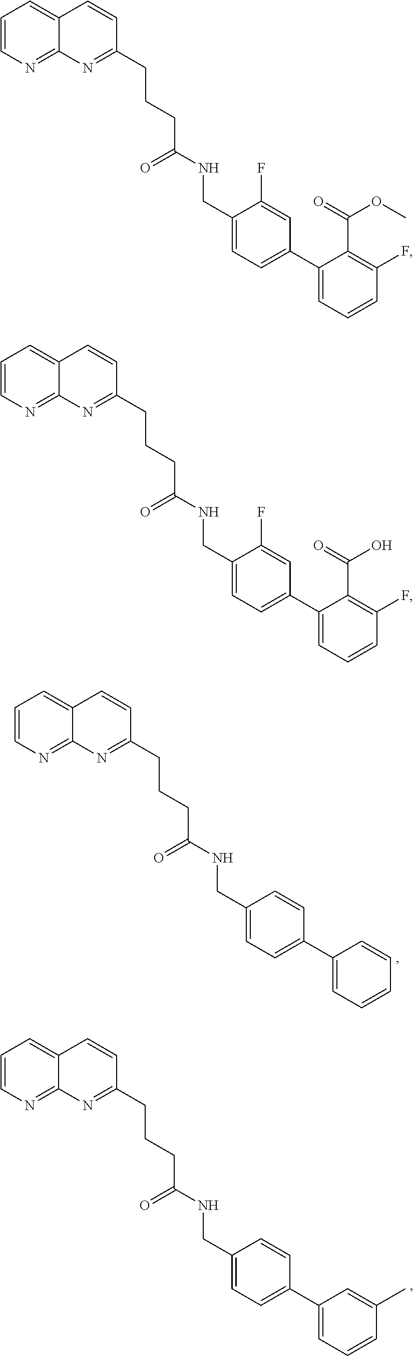 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1]

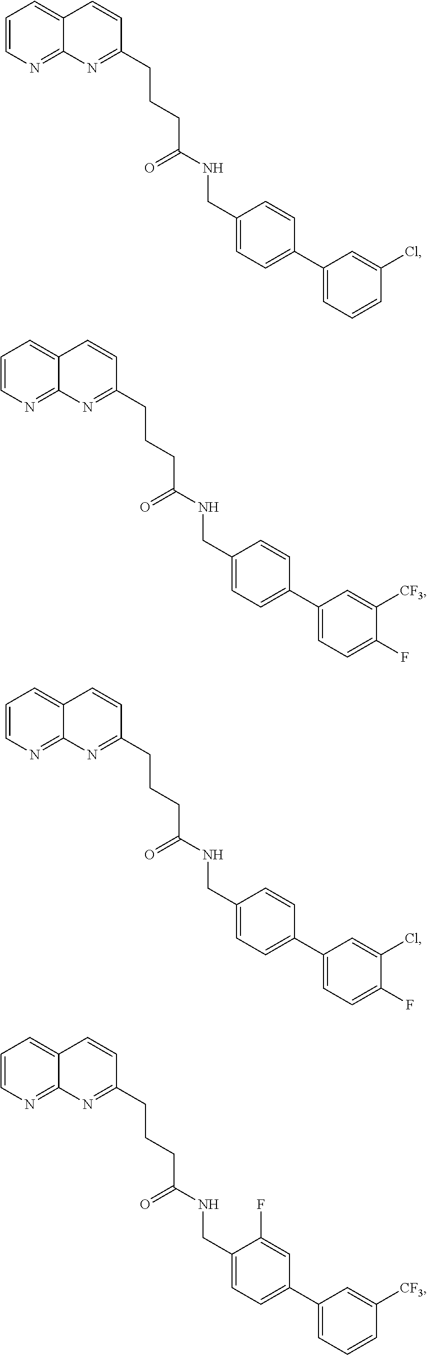 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1 ; O=C(CCCc1ccc2cccnc2n1)NCc1ccc(-c2cccc(C(F)(F)F)c2)cc1F]

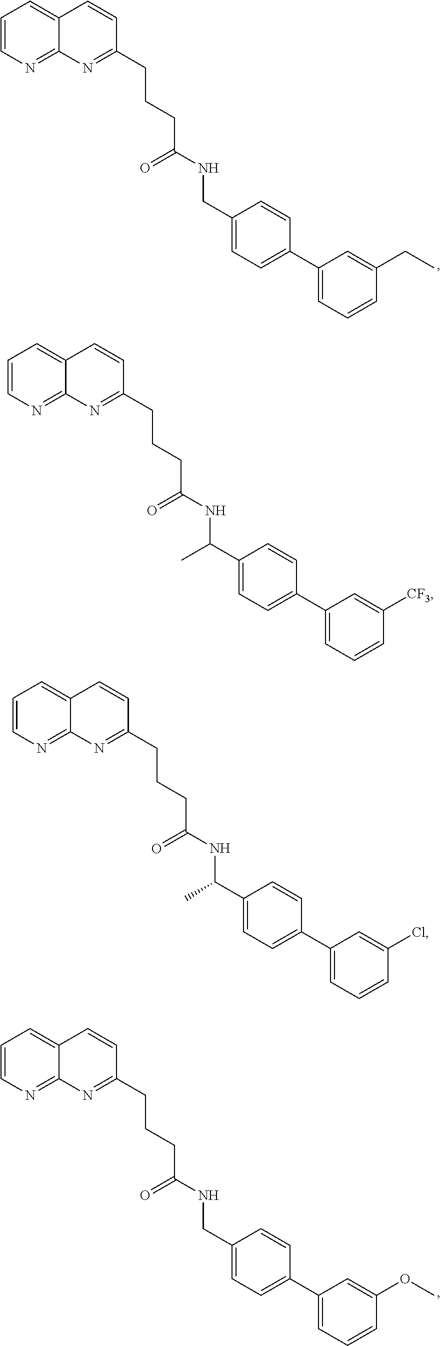 [SMILES: CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1 ; COc1cccc(-c2ccc(CNC(=O)CCCc3ccc4cccnc4n3)cc2)c1]

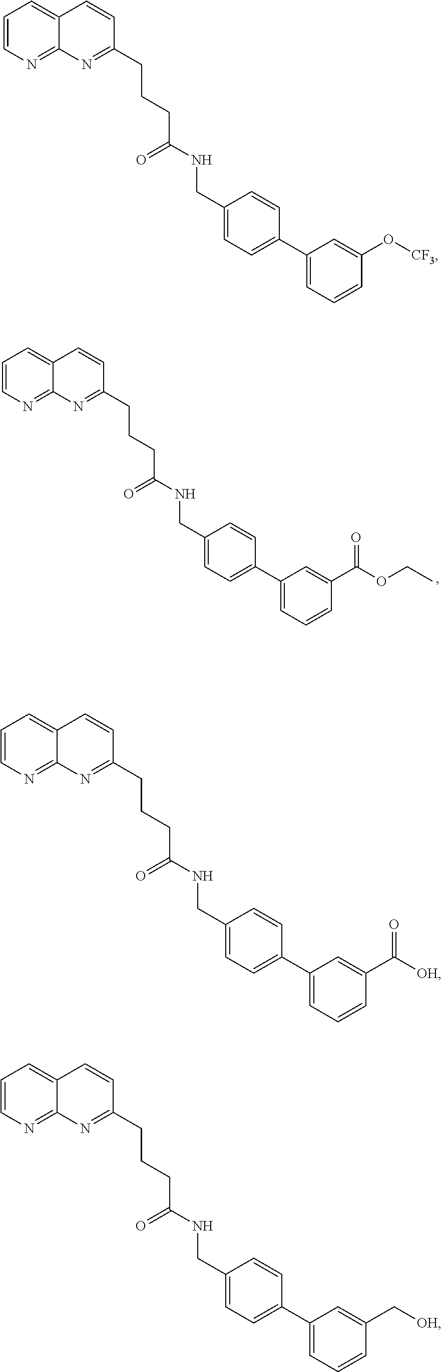 [SMILES: O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1 ; O=C(CCCc1ccc2cccnc2n1)NCc1ccc(-c2cccc(CO)c2)cc1]

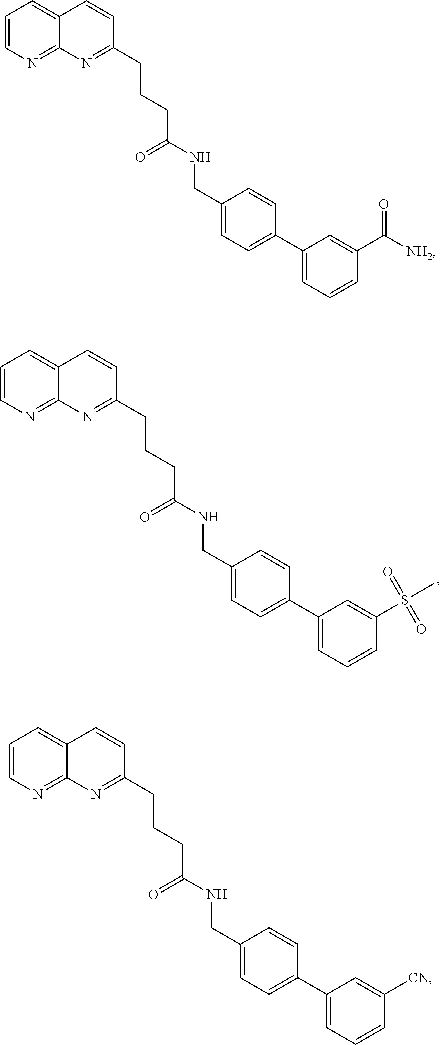 [SMILES: NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1]

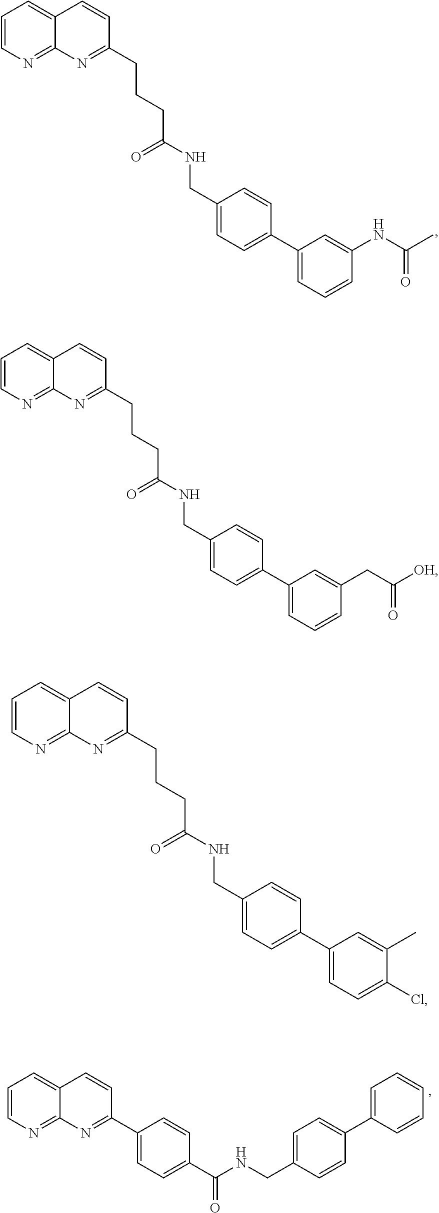 [SMILES: CC(=O)Nc1cccc(-c2ccc(CNC(=O)CCCc3ccc4cccnc4n3)cc2)c1 ; O=C(O)Cc1cccc(-c2ccc(CNC(=O)CCCc3ccc4cccnc4n3)cc2)c1 ; Cc1cc(-c2ccc(CNC(=O)CCCc3ccc4cccnc4n3)cc2)ccc1Cl ; O=C(NCc1ccc(-c2ccccc2)cc1)c1ccc(-c2ccc3cccnc3n2)cc1]

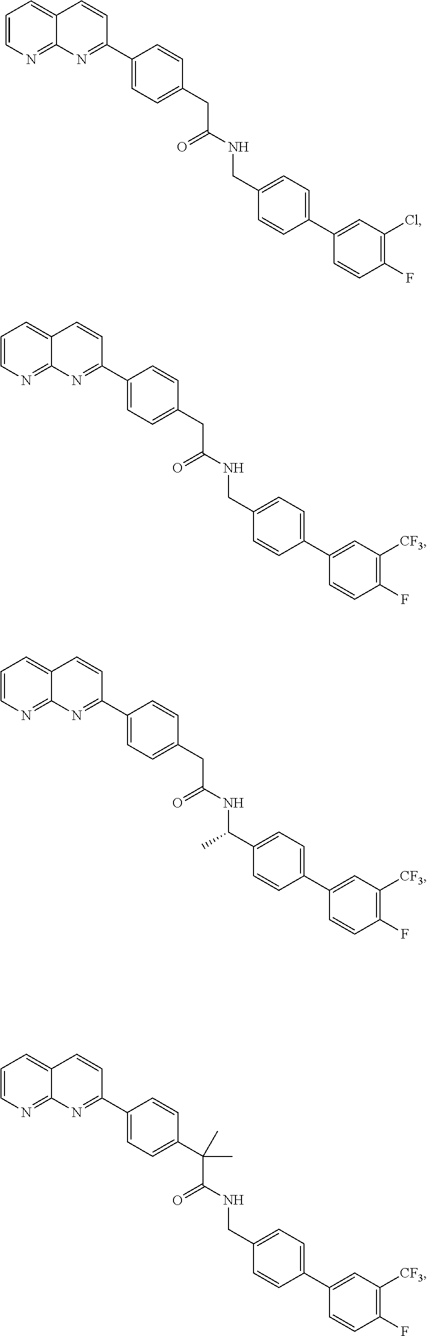 [SMILES: O=C(Cc1ccc(-c2ccc3cccnc3n2)cc1)NCc1ccc(-c2ccc(F)c(Cl)c2)cc1 ; O=C(Cc1ccc(-c2ccc3cccnc3n2)cc1)NCc1ccc(-c2ccc(F)c(C(F)(F)F)c2)cc1 ; C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1]

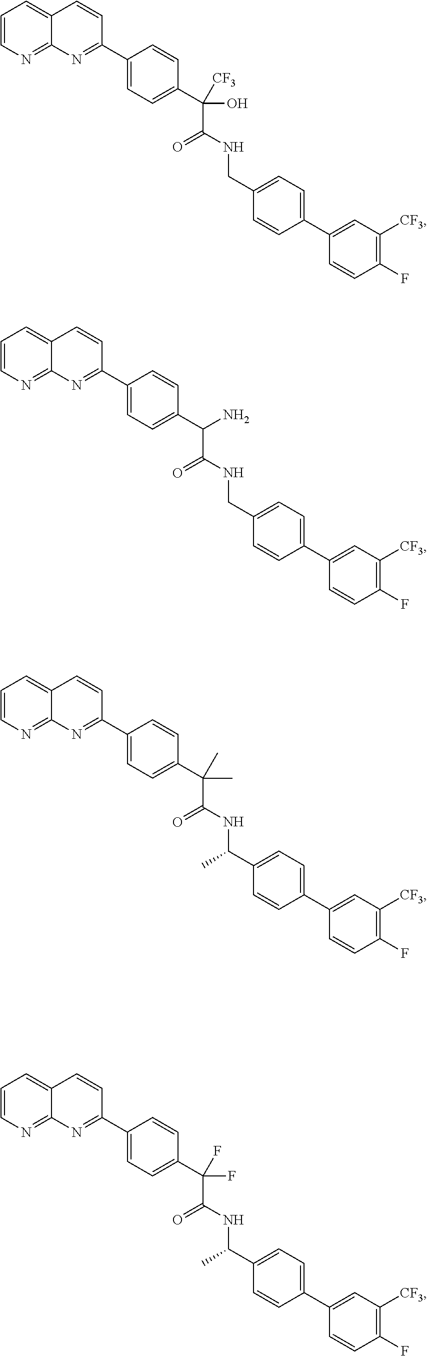 [SMILES: O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1 ; C[C@H](NC(=O)C(F)(F)c1ccc(-c2ccc3cccnc3n2)cc1)c1ccc(-c2ccc(F)c(C(F)(F)F)c2)cc1]

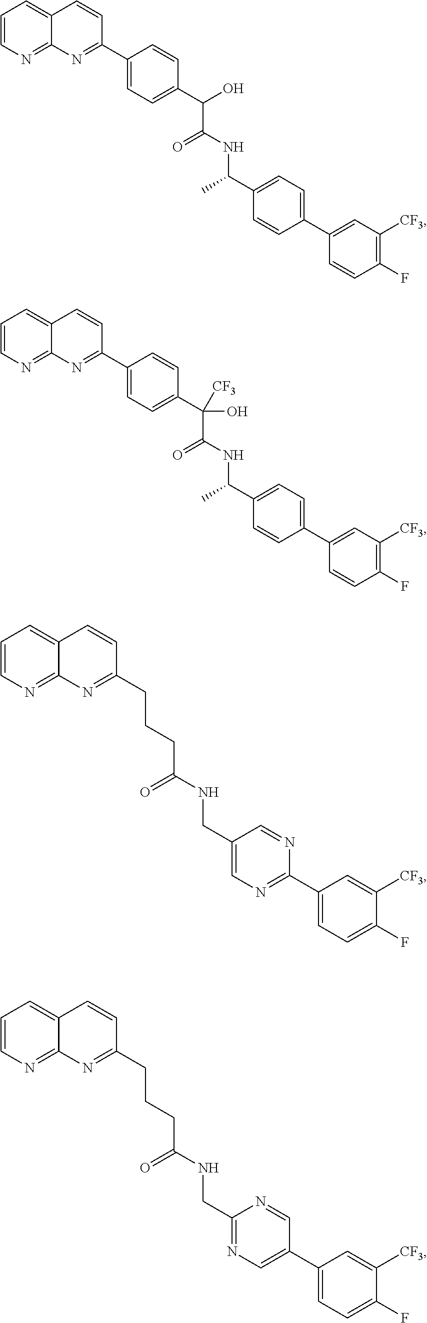 [SMILES: C[C@H](NC(=O)C(O)c1ccc(-c2ccc3cccnc3n2)cc1)c1ccc(-c2ccc(F)c(C(F)(F)F)c2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1 ; O=C(CCCc1ccc2cccnc2n1)NCc1ncc(-c2ccc(F)c(C(F)(F)F)c2)cn1]

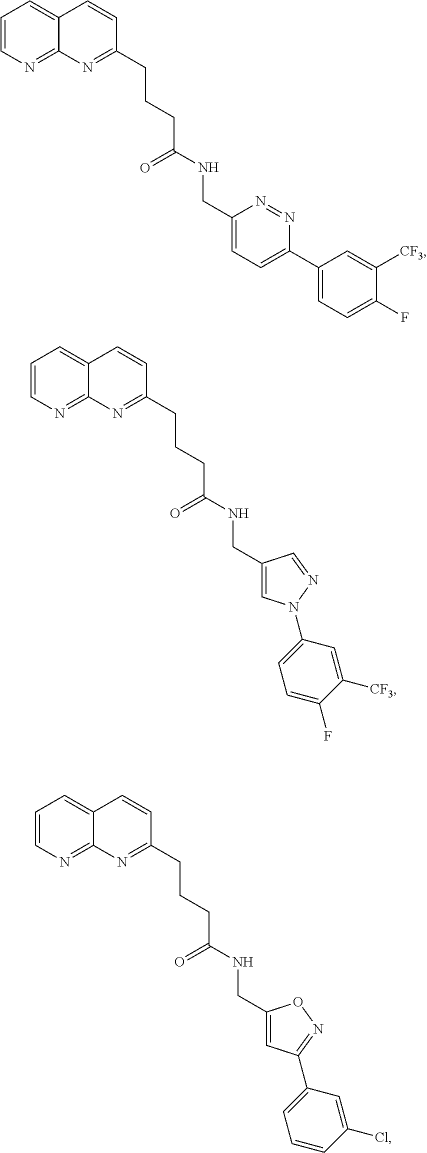 [SMILES: O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1]

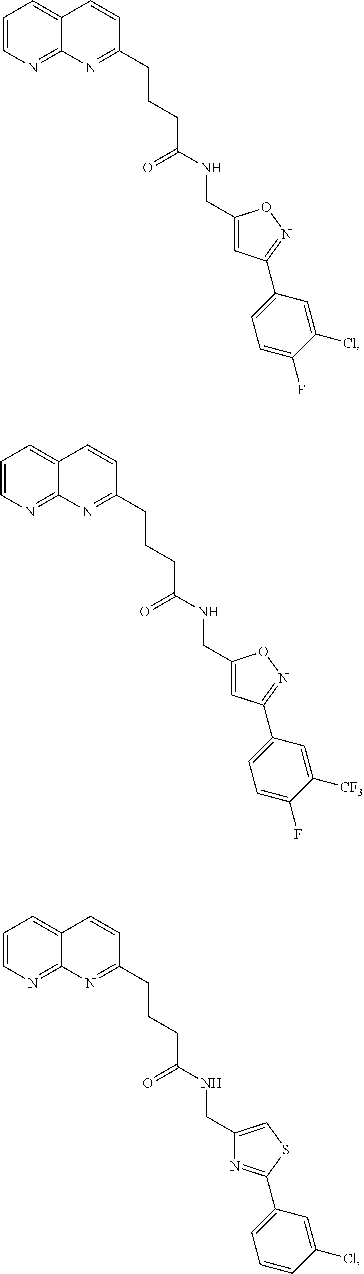 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1]

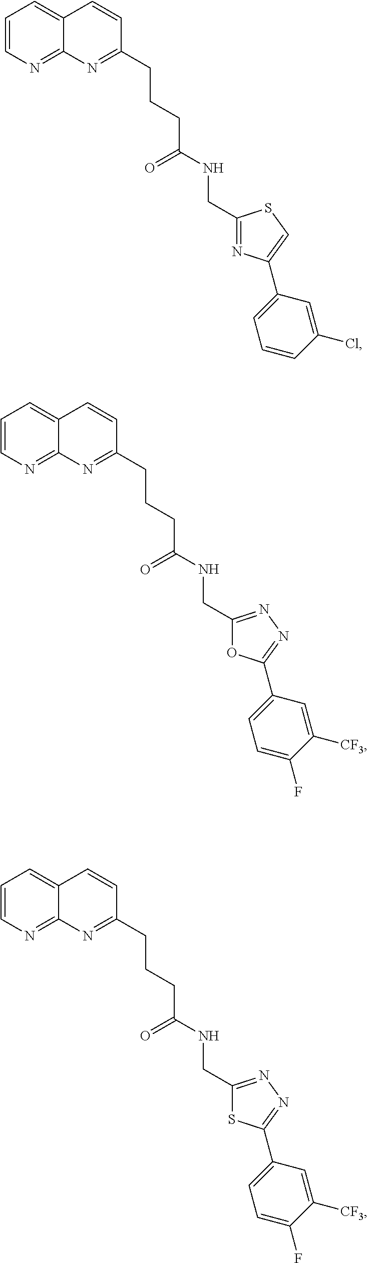 [SMILES: O=C(CCCc1ccc2cccnc2n1)NCc1nc(-c2cccc(Cl)c2)cs1 ; O=C(CCCc1ccc2cccnc2n1)NCc1nnc(-c2ccc(F)c(C(F)(F)F)c2)o1 ; O=C(CCCc1ccc2cccnc2n1)NCc1nnc(-c2ccc(F)c(C(F)(F)F)c2)s1]

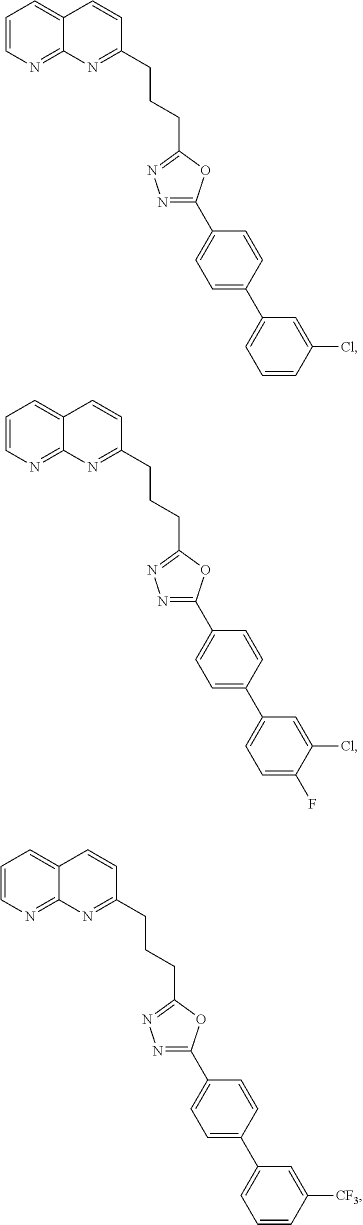 [SMILES: Clc1cccc(-c2ccc(-c3nnc(CCCc4ccc5cccnc5n4)o3)cc2)c1 ; Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1]

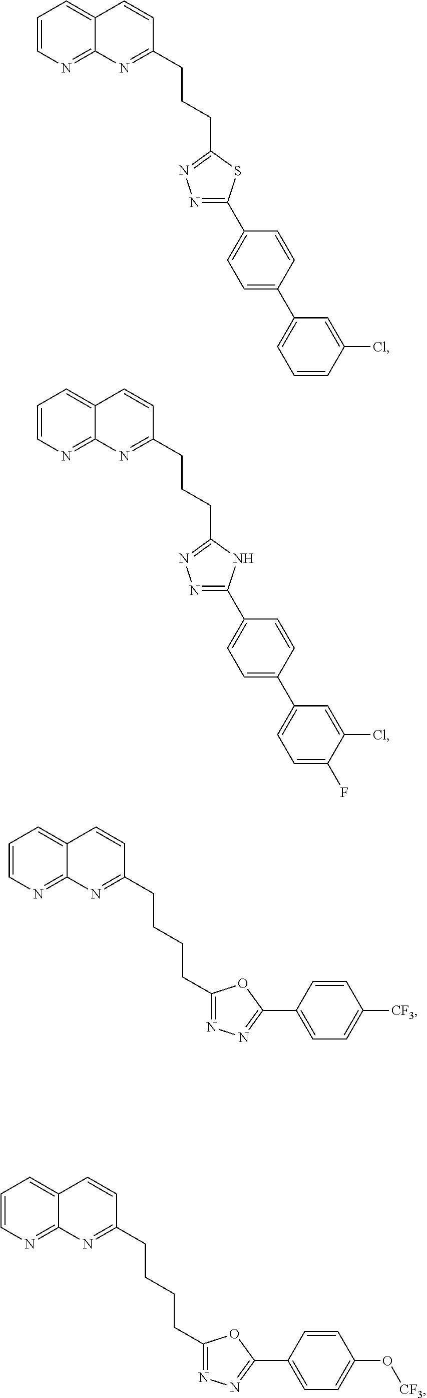 [SMILES: Clc1cccc(-c2ccc(-c3nnc(CCCc4ccc5cccnc5n4)s3)cc2)c1 ; Fc1ccc(-c2ccc(-c3nnc(CCCc4ccc5cccnc5n4)[nH]3)cc2)cc1Cl ; FC(F)(F)c1ccc(-c2nnc(CCCCc3ccc4cccnc4n3)o2)cc1 ; FC(F)(F)Oc1ccc(-c2nnc(CCCCc3ccc4cccnc4n3)o2)cc1]

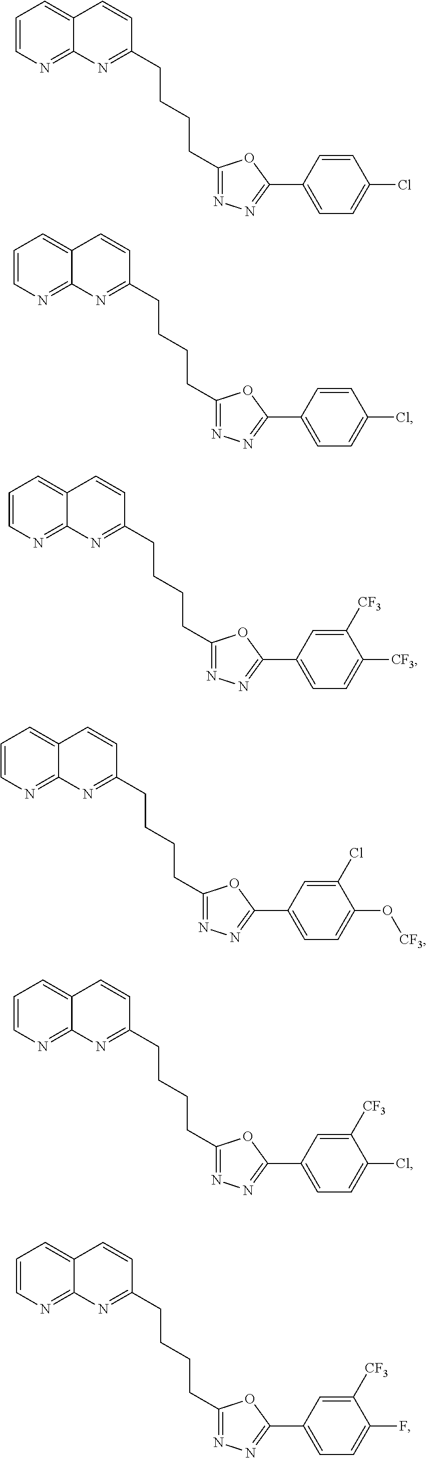 [SMILES: Clc1ccc(-c2nnc(CCCCc3ccc4cccnc4n3)o2)cc1 ; Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F ; FC(F)(F)Oc1ccc(-c2nnc(CCCCc3ccc4cccnc4n3)o2)cc1Cl ; FC(F)(F)c1cc(-c2nnc(CCCCc3ccc4cccnc4n3)o2)ccc1Cl ; Fc1ccc(-c2nnc(CCCCc3ccc4cccnc4n3)o2)cc1C(F)(F)F]

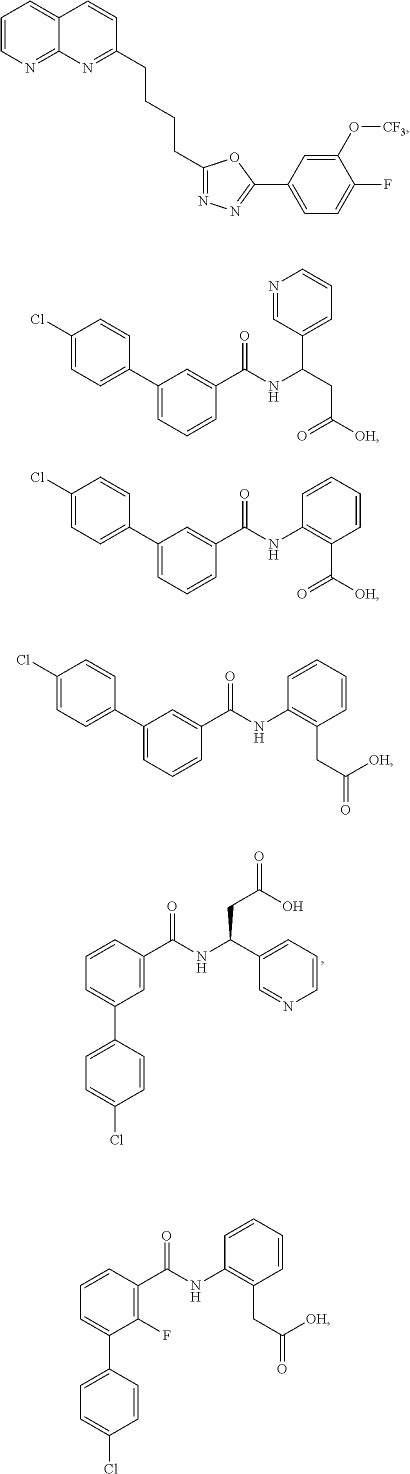 [SMILES: Fc1ccc(-c2nnc(CCCCc3ccc4cccnc4n3)o2)cc1OC(F)(F)F ; O=C(O)CC(NC(=O)c1cccc(-c2ccc(Cl)cc2)c1)c1cccnc1 ; O=C(Nc1ccccc1C(=O)O)c1cccc(-c2ccc(Cl)cc2)c1 ; O=C(O)Cc1ccccc1NC(=O)c1cccc(-c2ccc(Cl)cc2)c1 ; O=C(O)C[C@H](NC(=O)c1cccc(-c2ccc(Cl)cc2)c1)c1cccnc1 ; O=C(O)Cc1ccccc1NC(=O)c1cccc(-c2ccc(Cl)cc2)c1F]

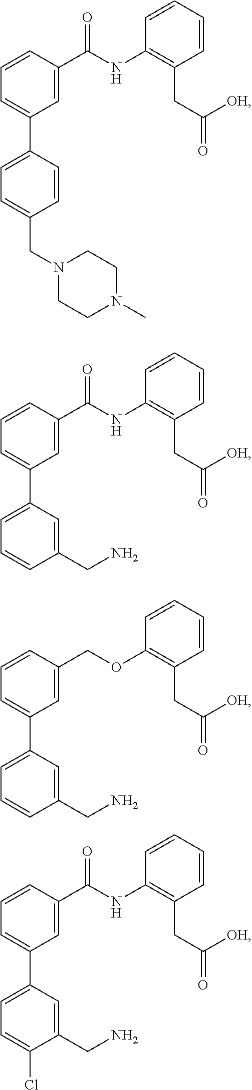

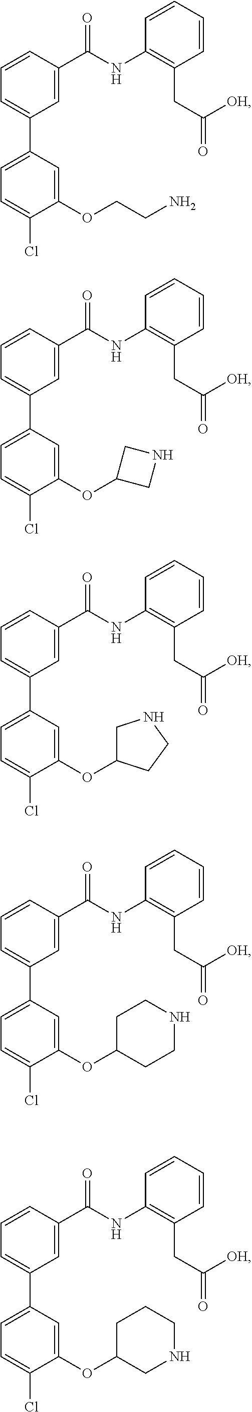 [SMILES: NCCOc1cc(-c2cccc(C(=O)Nc3ccccc3CC(=O)O)c2)ccc1Cl ; O=C(O)Cc1ccccc1NC(=O)c1cccc(-c2ccc(Cl)c(OC3CNC3)c2)c1 ; O=C(O)Cc1ccccc1NC(=O)c1cccc(-c2ccc(Cl)c(OC3CCNC3)c2)c1 ; O=C(O)Cc1ccccc1NC(=O)c1cccc(-c2ccc(Cl)c(OC3CCNCC3)c2)c1 ; O=C(O)Cc1ccccc1NC(=O)c1cccc(-c2ccc(Cl)c(OC3CCCNC3)c2)c1]

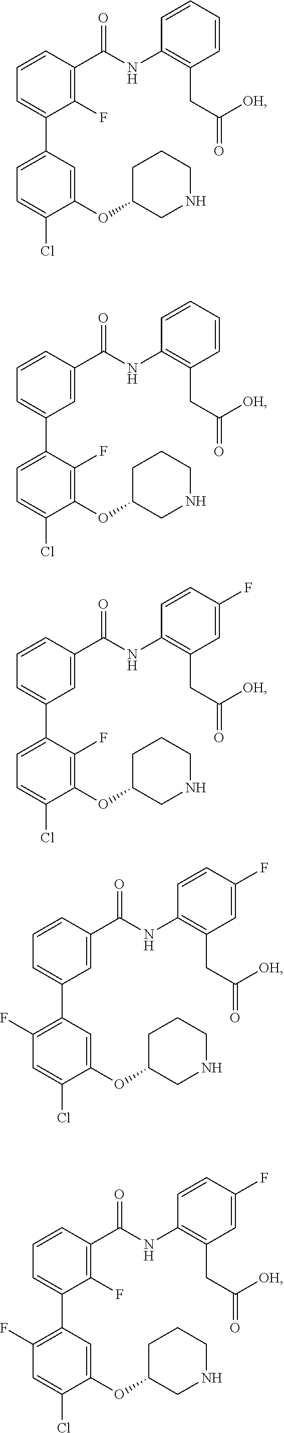 [SMILES: O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1 ; O=C(O)Cc1cc(F)ccc1NC(=O)c1cccc(-c2cc(O[C@@H]3CCCNC3)c(Cl)cc2F)c1F]

and combinations thereof.
Statement 58. A method of Statement 55 or Statement 56, wherein the succinate/succinate receptor 1 inhibitor has the following structure or a succinate salt thereof:

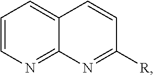

where R is selected from the group consisting of a hydrogen atom, an alkyl group, COOR′ group, and

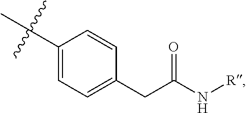

where R′ is an alkyl group and R″ is selected from the group consisting of a hydrogen atom, an alkyl group, and

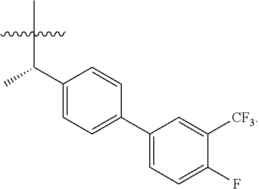 [SMILES: *[C@@H](C)c1ccc(-c2ccc(F)c(C(F)(F)F)c2)cc1]

Statement 59. A method of Statement 58, wherein the succinate/succinate receptor 1 inhibitor has the following structure:

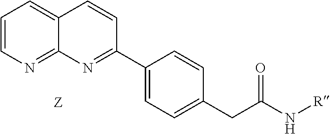

where R″ is selected from the group consisting of a hydrogen atom, an alkyl group, and

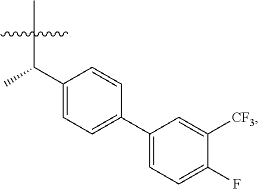 [SMILES: *[C@@H](C)c1ccc(-c2ccc(F)c(C(F)(F)F)c2)cc1]

and where Z is optional and when present, is a succinate group.
Statement 60. A method of Statement 59, wherein the succinate/succinate receptor 1 inhibitor is selected from the group consisting of

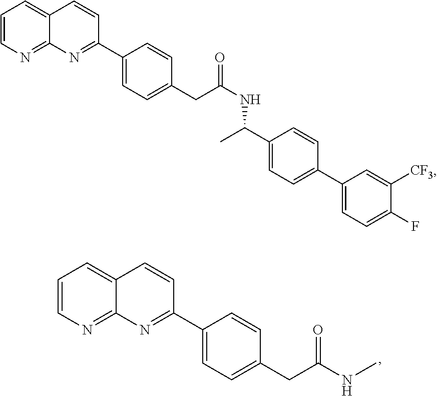

and succinate salts thereof.
Statement 61. A method of Statement 60, wherein the succinate/succinate receptor 1 inhibitor is

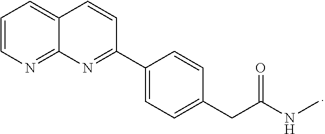 [SMILES: CNC(=O)Cc1ccc(-c2ccc3cccnc3n2)cc1]

Statement 62. A composition (e.g., a gel composition) comprising a succinate/succinate receptor 1 inhibitor and one or more polymers, wherein the inhibitor of succinate/succinate receptor has the following structure or a succinate salt thereof:

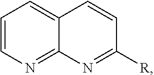

where K is selected from the group consisting of a hydrogen atom, an alkyl group, COOR′ group, and

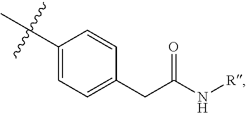

where R′ is an alkyl group and R″ is selected from the group consisting of a hydrogen atom, an alkyl group, and

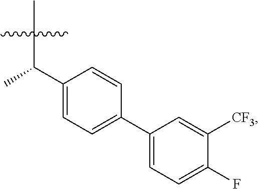 [SMILES: *[C@@H](C)c1ccc(-c2ccc(F)c(C(F)(F)F)c2)cc1]

with the proviso the composition is not a thin film having a thickness of from 0.05 mm to 0.4 mm.

Statement 63. A composition of Statement 62, wherein the succinate/succinate receptor 1 inhibitor has the following structure:

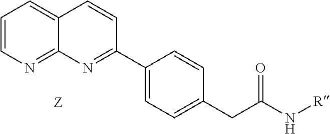

where R″ is selected from the group consisting of a hydrogen atom, an alkyl group, and

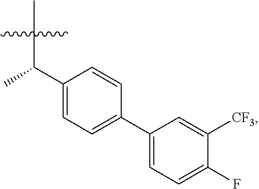 [SMILES: *[C@@H](C)c1ccc(-c2ccc(F)c(C(F)(F)F)c2)cc1]

and where Z is optional and when present, is a succinate group.
Statement 64. A composition of Statement 63, wherein the succinate/succinate receptor 1 inhibitor is selected from the group consisting of

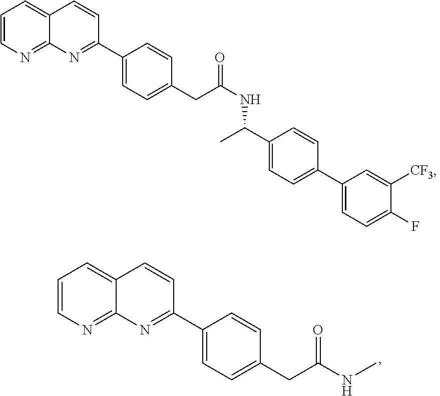

and succinate salts thereof.
Statement 65. The composition of Statement 64, wherein the succinate/succinate receptor 1 inhibitor is

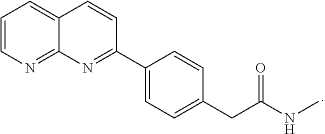 [SMILES: CNC(=O)Cc1ccc(-c2ccc3cccnc3n2)cc1]

Statement 66. A composition of Statement 65, wherein

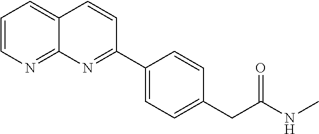 [SMILES: CNC(=O)Cc1ccc(-c2ccc3cccnc3n2)cc1]

has a concentration of 1-10 mg/mL.
Statement 67. A composition of Statement 66, wherein the concentration is 3-7 mg/mL.
Statement 68. A composition of Statement 67, wherein the concentration is 4-6 mg/mL.
Statement 69. A composition of Statement 68, wherein the concentration is around or about 5 mg/mL or is 5 mg/mL.
Statement 70. A composition of Statement 69, wherein the concentration is 5 mg/mL.
Statement 71. A composition according to any one of Statements 62-70, wherein the one or more polymers are chosen from poly lactic glycolic acid (PLGA), polycaprolactone (PCL), poly-ε-caprolactone ester terminated, poly(D,L-lactide-co-glycolide, poly(D,L-lactide-co-glycolide ester terminated, poly(D,L-lactide, poly(D,L-lactide) ester terminated, chitosan, starch, polylactic acid, alginate, and combinations thereof.
Statement 72. A composition of Statement 71, wherein the one or more polymers are chosen from PLGA, poly-ε-caprolactone ester terminated, poly(D,L-lactide) ester-terminated, and poly(D,L-lactide-co-glycolide) ester terminated.
Statement 73. A composition according to any one of Statements 62-72, further comprising a solvent.
Statement 74. A composition of Statement 73, wherein the solvent is benzyl benzoate, N-methyl-2-pyrrolidinone, or a combination thereof.
Statement 75. A composition according to any one of Statements 62-70, wherein the succinate/succinate receptor 1 inhibitor is

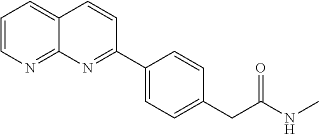 [SMILES: CNC(=O)Cc1ccc(-c2ccc3cccnc3n2)cc1]

the polymer is PLGA, and the composition further comprises a solvent, wherein the solvent is a 30% solution of benzyl benzoate in N-methyl-2-pyrrolidinone.
Statement 76. A composition of Statement 75, wherein the succinate/succinate receptor 1 inhibitor is

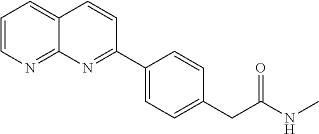 [SMILES: CNC(=O)Cc1ccc(-c2ccc3cccnc3n2)cc1]

having a concentration of 5.0 mg/mL, the polymer is PLGA having a concentration of 10% w/v, and the solvent is a 30% solution of benzyl benzoate in N-methyl-2-pyrrolidinone.
Statement 77. A composition (e.g., a gel composition) comprising a succinate/succinate receptor 1 inhibitor and one or more polymers, wherein the inhibitor of succinate/succinate receptor has the following structure or a succinate salt thereof:

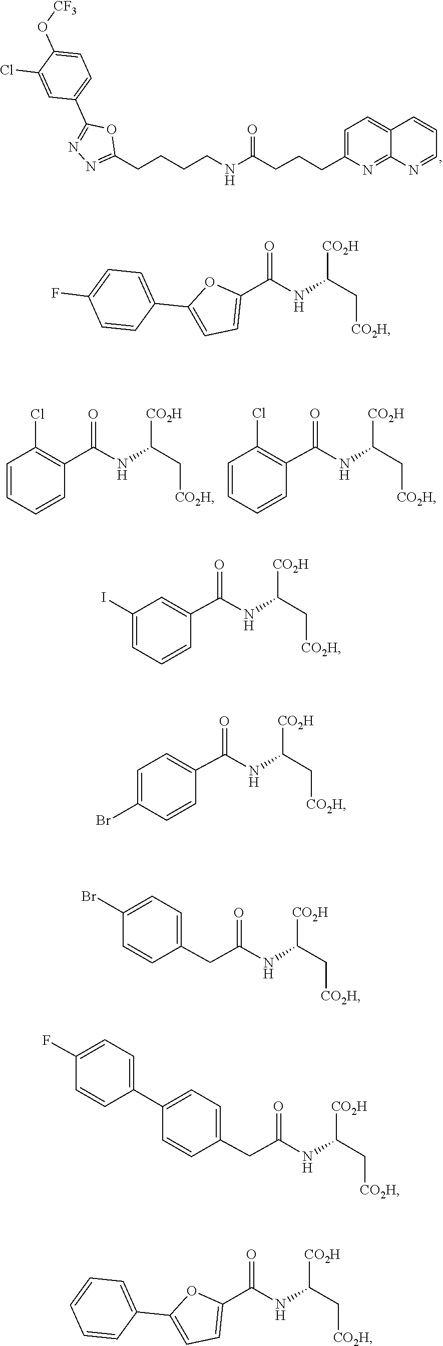

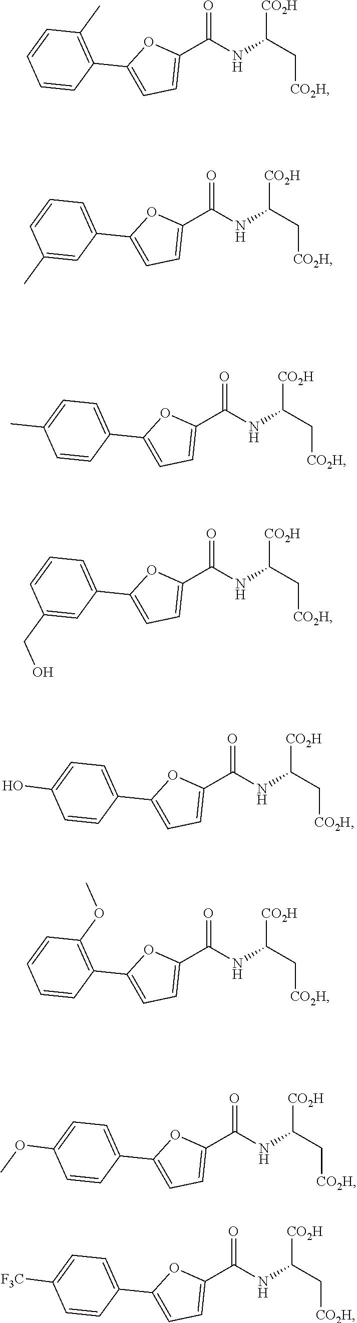

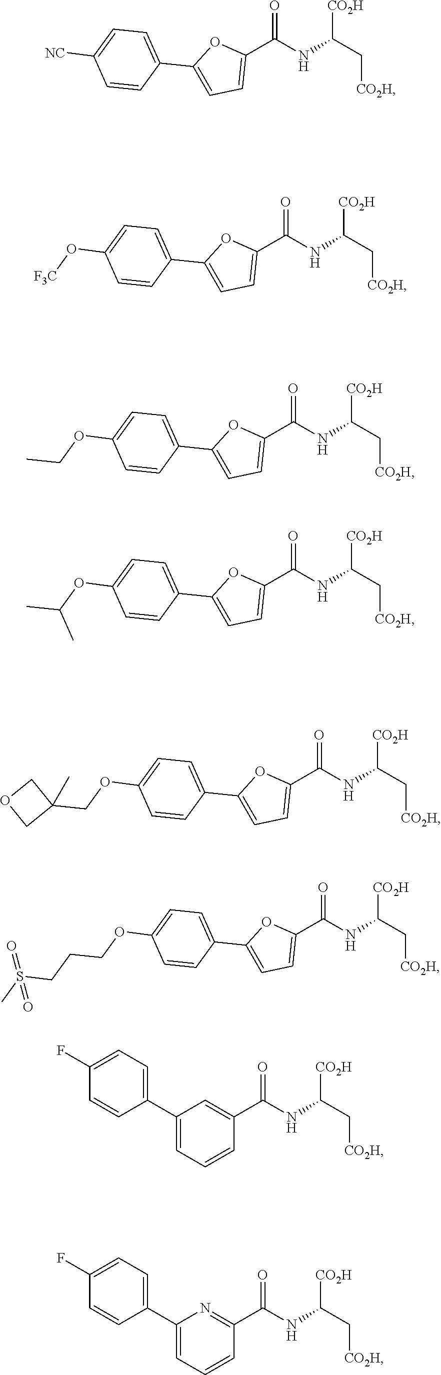

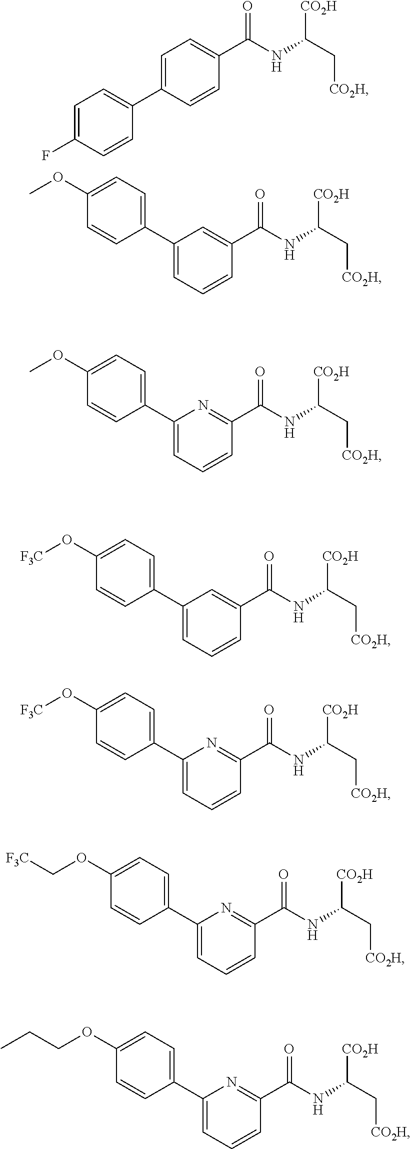

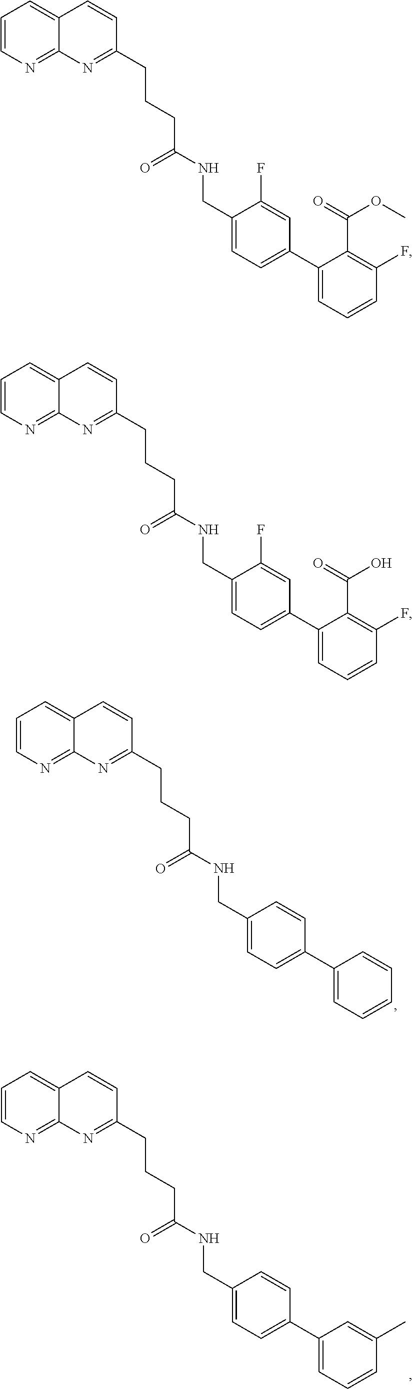 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1]

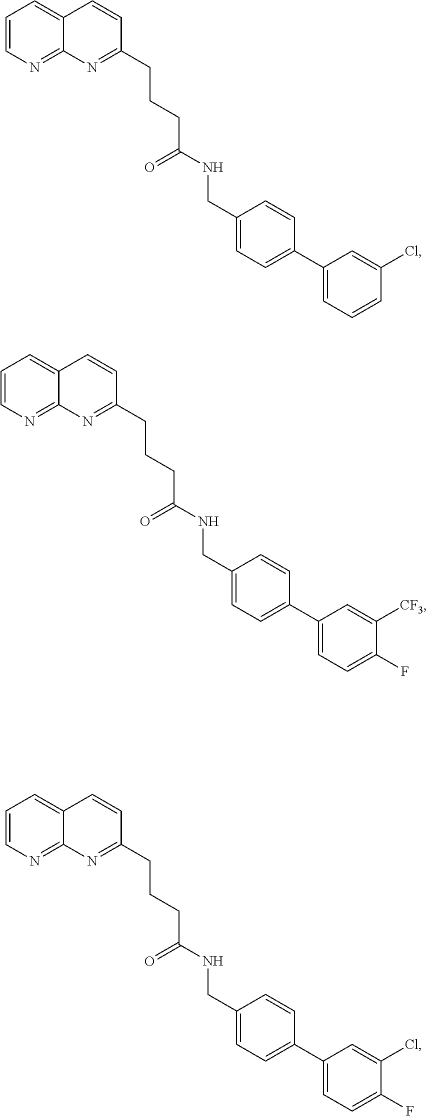 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1]

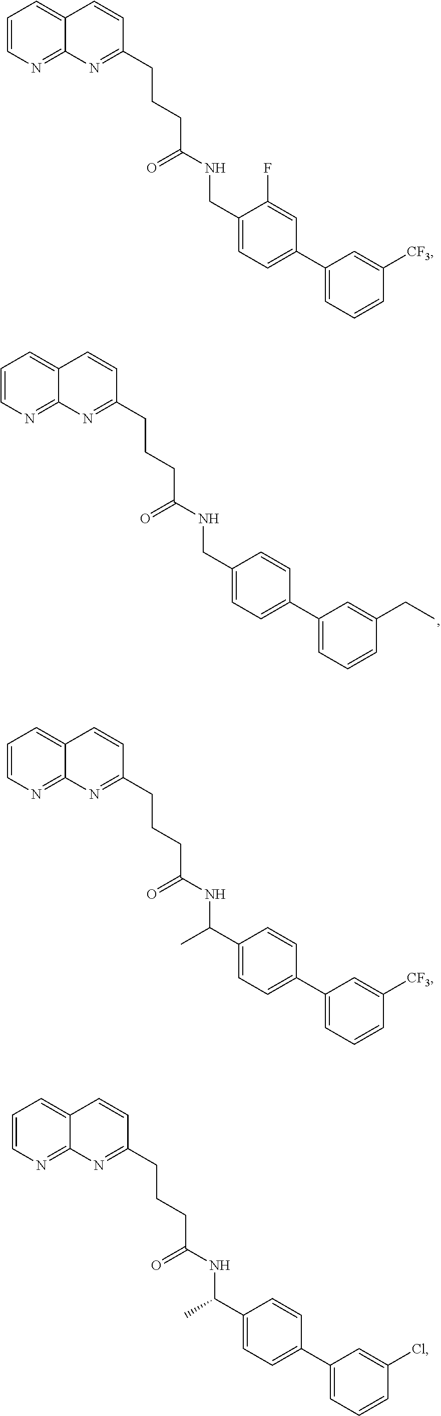 [SMILES: O=C(CCCc1ccc2cccnc2n1)NCc1ccc(-c2cccc(C(F)(F)F)c2)cc1F ; CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1]

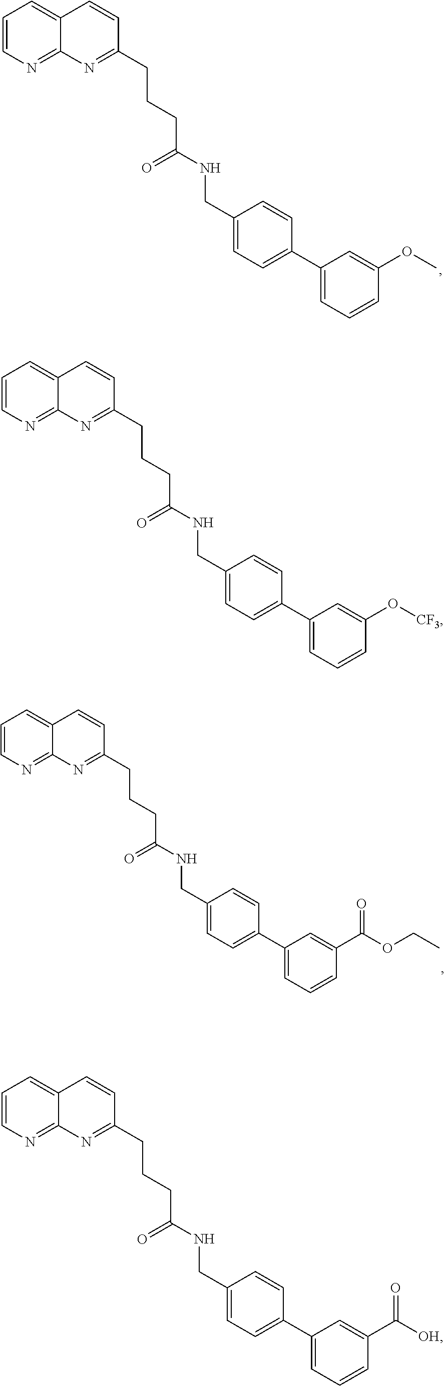 [SMILES: COc1cccc(-c2ccc(CNC(=O)CCCc3ccc4cccnc4n3)cc2)c1 ; O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1]

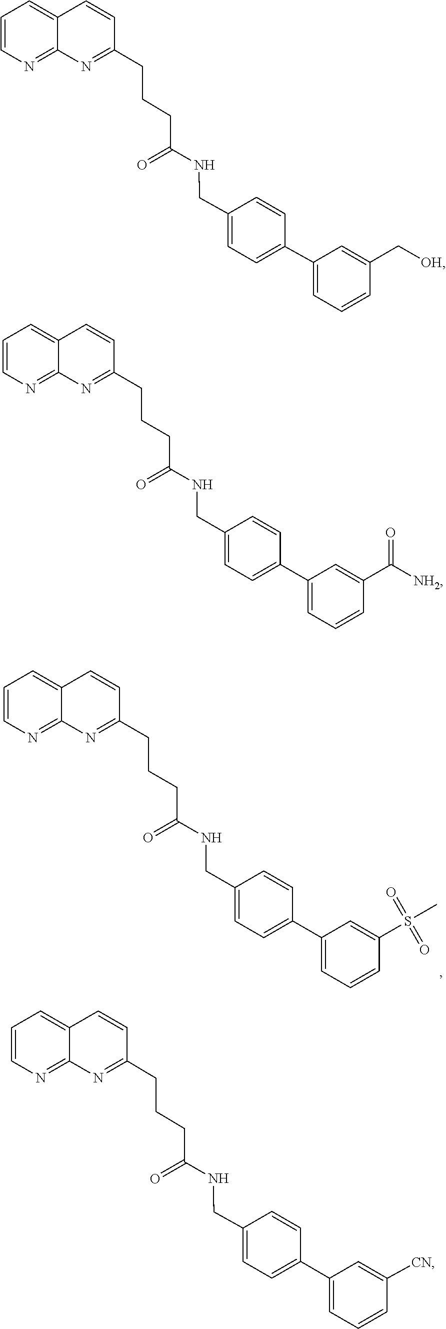 [SMILES: O=C(CCCc1ccc2cccnc2n1)NCc1ccc(-c2cccc(CO)c2)cc1 ; NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1]

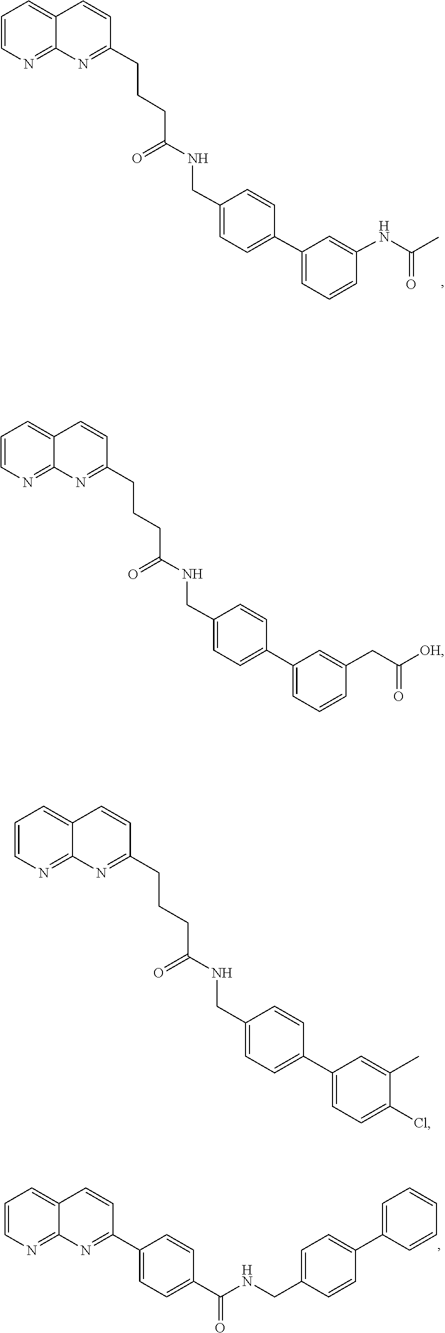 [SMILES: CC(=O)Nc1cccc(-c2ccc(CNC(=O)CCCc3ccc4cccnc4n3)cc2)c1 ; O=C(O)Cc1cccc(-c2ccc(CNC(=O)CCCc3ccc4cccnc4n3)cc2)c1 ; Cc1cc(-c2ccc(CNC(=O)CCCc3ccc4cccnc4n3)cc2)ccc1Cl ; O=C(NCc1ccc(-c2ccccc2)cc1)c1ccc(-c2ccc3cccnc3n2)cc1]

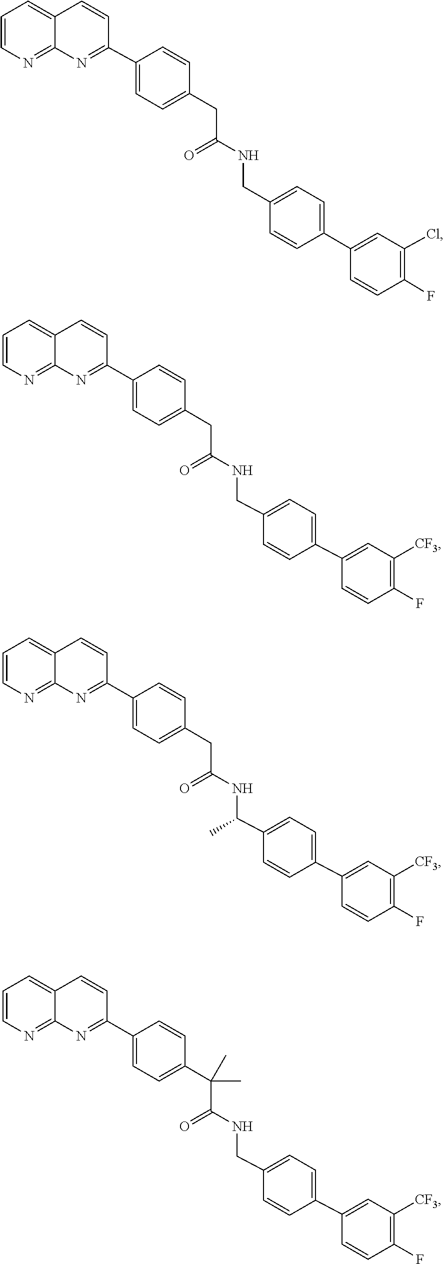 [SMILES: O=C(Cc1ccc(-c2ccc3cccnc3n2)cc1)NCc1ccc(-c2ccc(F)c(Cl)c2)cc1 ; O=C(Cc1ccc(-c2ccc3cccnc3n2)cc1)NCc1ccc(-c2ccc(F)c(C(F)(F)F)c2)cc1 ; C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1]

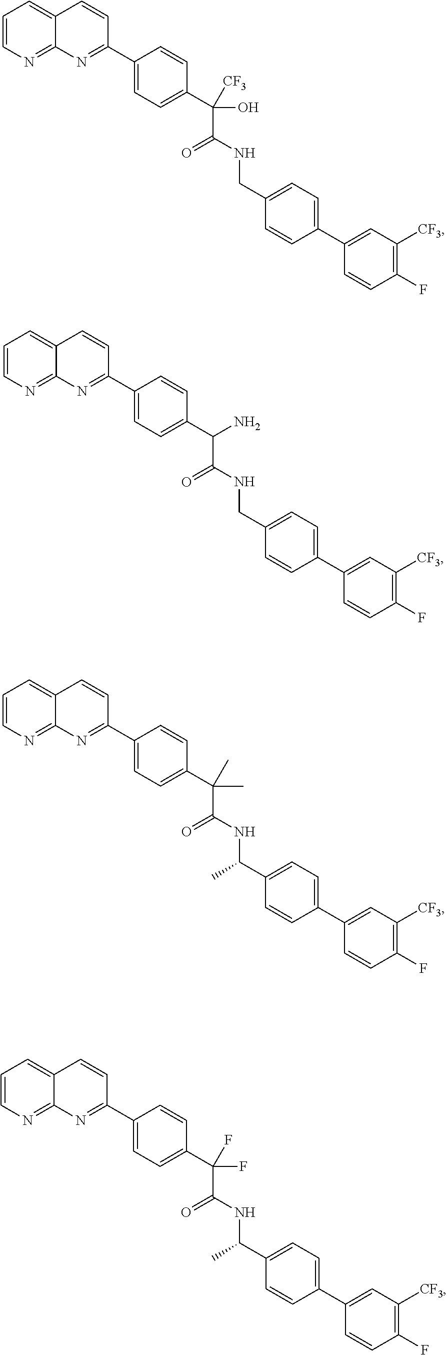 [SMILES: O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1 ; C[C@H](NC(=O)C(F)(F)c1ccc(-c2ccc3cccnc3n2)cc1)c1ccc(-c2ccc(F)c(C(F)(F)F)c2)cc1]

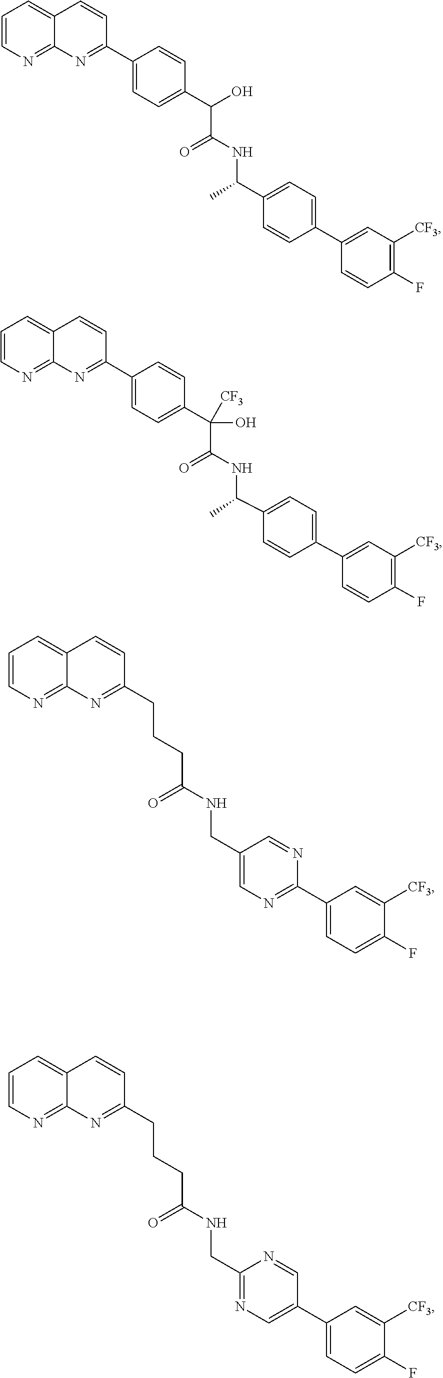 [SMILES: C[C@H](NC(=O)C(O)c1ccc(-c2ccc3cccnc3n2)cc1)c1ccc(-c2ccc(F)c(C(F)(F)F)c2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1 ; O=C(CCCc1ccc2cccnc2n1)NCc1ncc(-c2ccc(F)c(C(F)(F)F)c2)cn1]

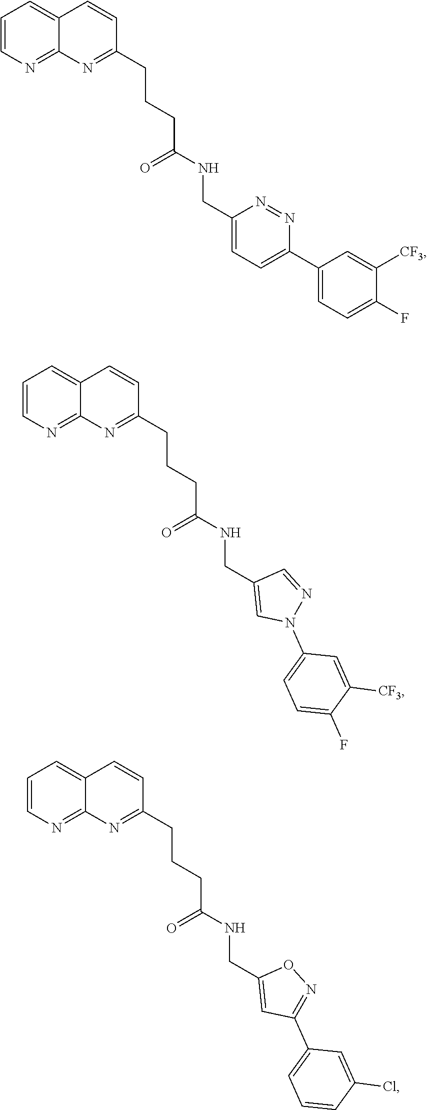 [SMILES: O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1]

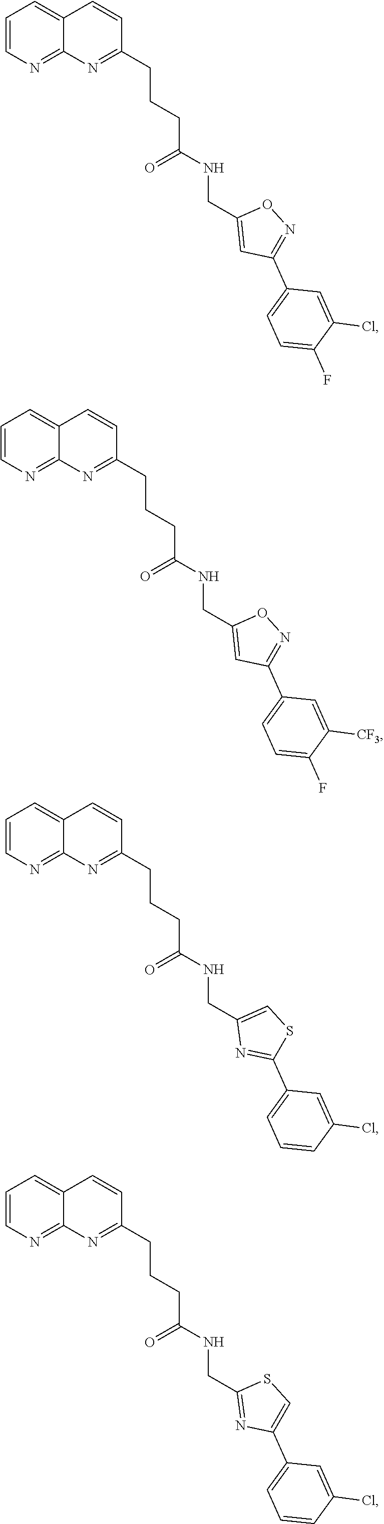 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1 ; O=C(CCCc1ccc2cccnc2n1)NCc1nc(-c2cccc(Cl)c2)cs1]

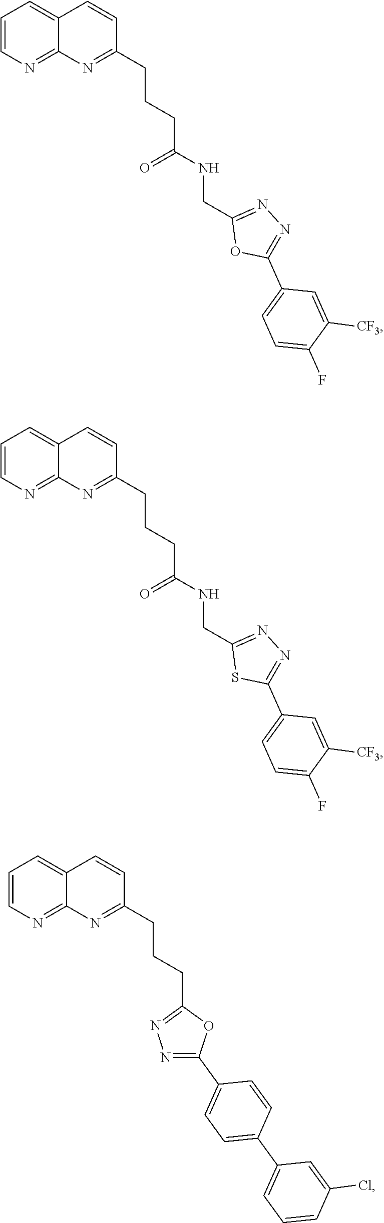 [SMILES: O=C(CCCc1ccc2cccnc2n1)NCc1nnc(-c2ccc(F)c(C(F)(F)F)c2)o1 ; O=C(CCCc1ccc2cccnc2n1)NCc1nnc(-c2ccc(F)c(C(F)(F)F)c2)s1 ; Clc1cccc(-c2ccc(-c3nnc(CCCc4ccc5cccnc5n4)o3)cc2)c1]

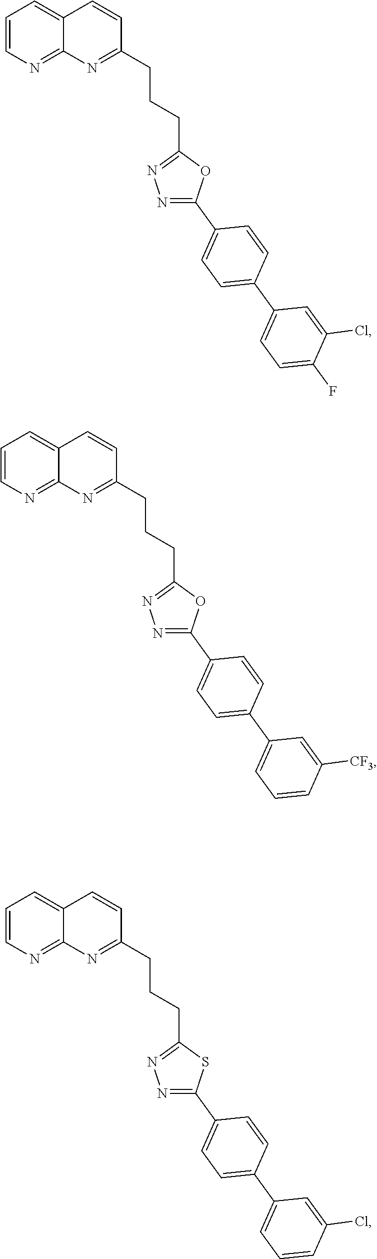 [SMILES: Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1 ; Clc1cccc(-c2ccc(-c3nnc(CCCc4ccc5cccnc5n4)s3)cc2)c1]

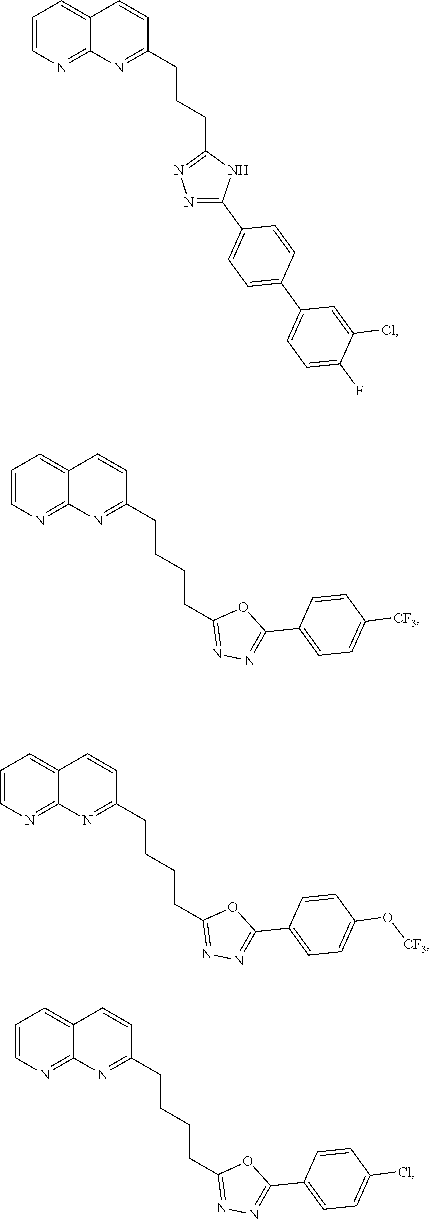 [SMILES: Fc1ccc(-c2ccc(-c3nnc(CCCc4ccc5cccnc5n4)[nH]3)cc2)cc1Cl ; FC(F)(F)c1ccc(-c2nnc(CCCCc3ccc4cccnc4n3)o2)cc1 ; FC(F)(F)Oc1ccc(-c2nnc(CCCCc3ccc4cccnc4n3)o2)cc1 ; Clc1ccc(-c2nnc(CCCCc3ccc4cccnc4n3)o2)cc1]

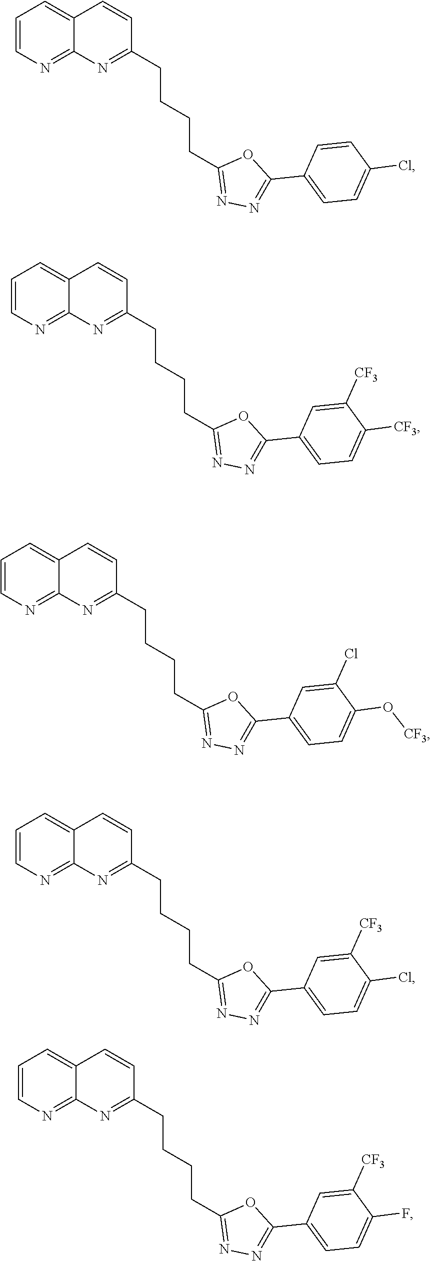 [SMILES: Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F ; FC(F)(F)Oc1ccc(-c2nnc(CCCCc3ccc4cccnc4n3)o2)cc1Cl ; FC(F)(F)c1cc(-c2nnc(CCCCc3ccc4cccnc4n3)o2)ccc1Cl ; Fc1ccc(-c2nnc(CCCCc3ccc4cccnc4n3)o2)cc1C(F)(F)F]

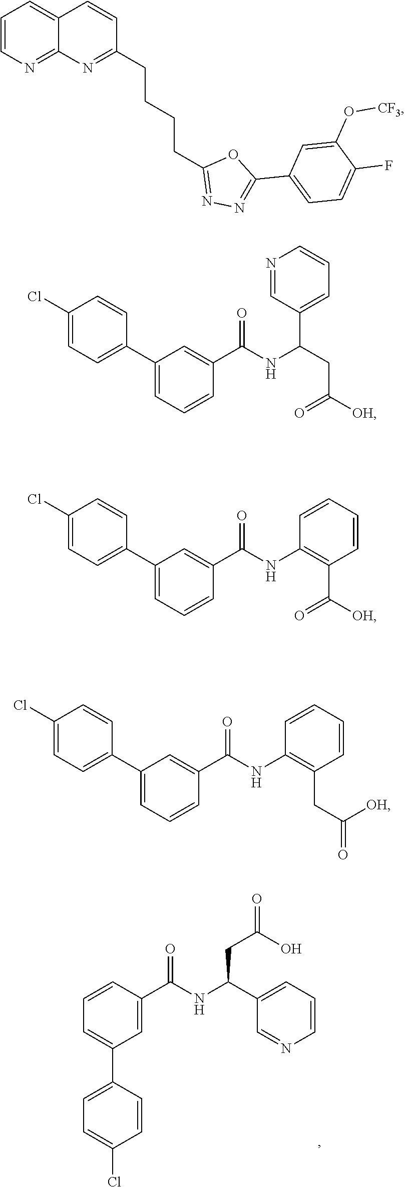

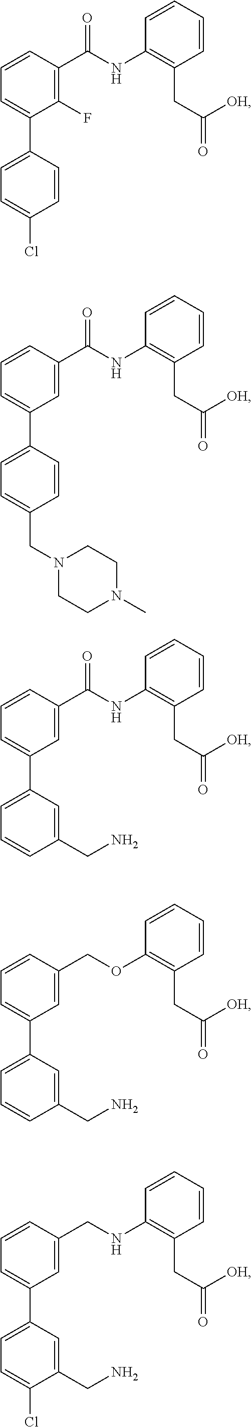

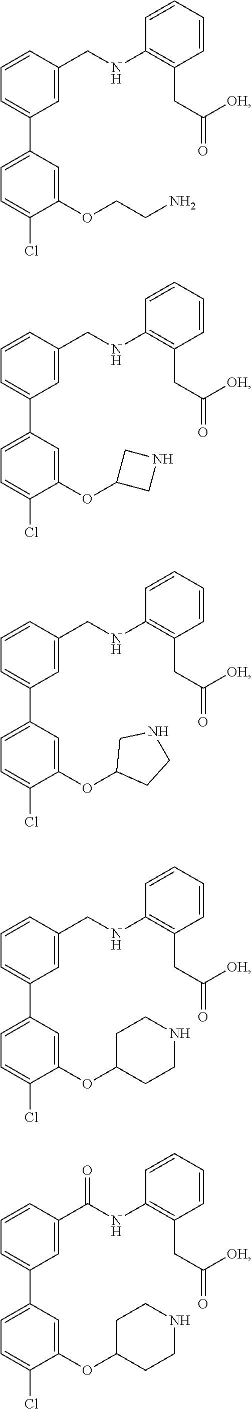 [SMILES: NCCOc1cc(-c2cccc(CNc3ccccc3CC(=O)O)c2)ccc1Cl ; O=C(O)Cc1ccccc1NCc1cccc(-c2ccc(Cl)c(OC3CNC3)c2)c1 ; O=C(O)Cc1ccccc1NCc1cccc(-c2ccc(Cl)c(OC3CCNC3)c2)c1 ; O=C(O)Cc1ccccc1NCc1cccc(-c2ccc(Cl)c(OC3CCNCC3)c2)c1 ; O=C(O)Cc1ccccc1NC(=O)c1cccc(-c2ccc(Cl)c(OC3CCNCC3)c2)c1]

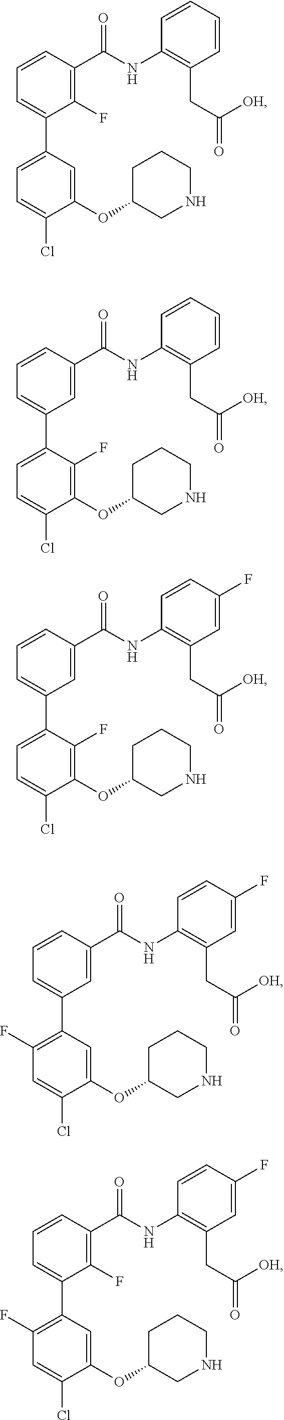 [SMILES: O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1 ; O=C(O)Cc1cc(F)ccc1NC(=O)c1cccc(-c2cc(O[C@@H]3CCCNC3)c(Cl)cc2F)c1F]

and combinations thereof.
Statement 78. A composition according to Statement 77, wherein the one or more polymers are chosen from poly lactic glycolic acid (PLGA), polycaprolactone (PCL), poly-6-caprolactone ester terminated, poly(D,L-lactide-co-glycolide, poly(D,L-lactide-co-glycolide ester terminated, poly(D,L-lactide, poly(D,L-lactide) ester terminated, chitosan, starch, polylactic acid, alginate, and combinations thereof.
Statement 79. A composition of Statement 77 or Statement 78, wherein the one or more polymers are chosen from PLGA, poly-ε-caprolactone ester terminated, poly(D,L-lactide) ester-terminated, and poly(D,L-lactide-co-glycolide) ester terminated.
Statement 80. A composition according to any one of Statements 77-79, further comprising a solvent.
Statement 81. A composition of Statement 81, wherein the solvent is benzyl benzoate, N-methyl-2-pyrrolidinone, or a combination thereof.

The following examples are intended to be illustrative and not restrictive.

### Example 1

The following example further describes synthesis and uses of compounds and compositions (e.g., gels) of the present disclosure.

Scheme 1 shows a synthetic route of Compound 7a and 7b.

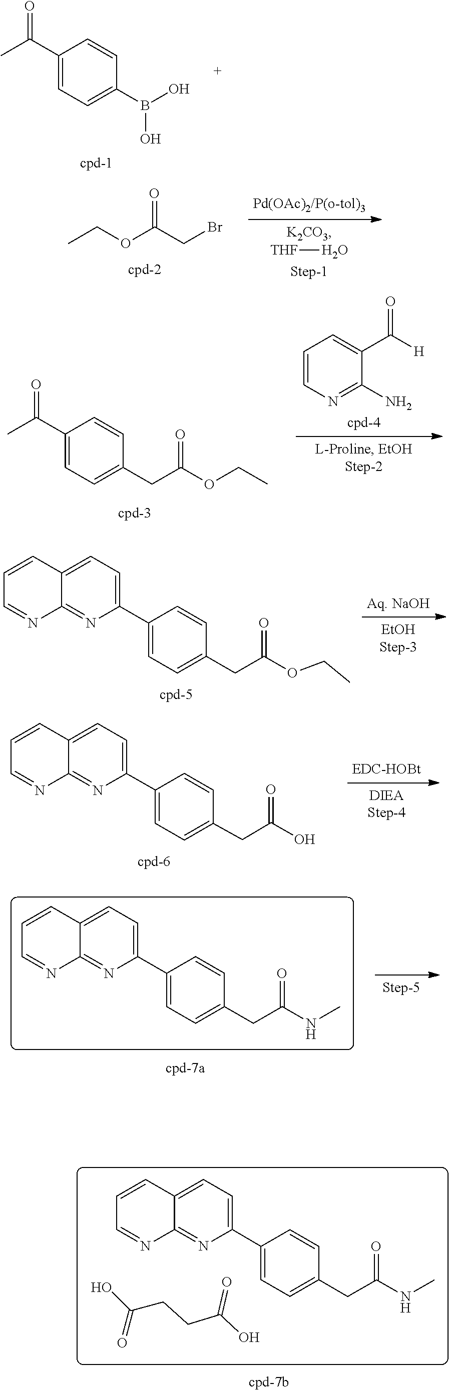

Scheme 1 correlates to the following steps:

| Step | Reaction scale/conditions | Results |
|---|---|---|
| 1 | cpd-1 (3.34 g, 1eq.), cpd-2 (3.93 g, 1.2 | Isolated cpd-3: 3.05 g (Y: 74%) |
| | eq).), Pd(OAc)₂ (3 mol %), P(o-tol)₃ (9 | Confirmed by 1HNMR analysis |
| | mol %), K₂CO₃ (5 eq), THF, H₂O, rt, 16 h | |
| 2 | cpd-3 (1.5 g, 1eq.), cpd-4 (0.88 g, 1eq.), | Isolated cpd-5: 1.1 g (Y: 52%) |
| | L-proline (1.0 eq.), EtOH, reflux, 12 h | Confirmed by 1HNMR analysis |
| 3 | cpd-5 (1.0 g, 1eq.), NaOH (3.0 eq), | Isolated cpd-6: 0.8 g (Y: 88%) |
| | EtOH, H₂O, rt, 3 h | Confirmed by 1HNMR analysis |
| 1 | cpd-1 (11.7 g, 1eq.), cpd-2 (9.94 g, 1.2 | Isolated cpd-3: 9.5 g (Y: 77%) |
| | eq).), Pd(OAc)₂ (3 mol %), P(o-tol)₃ (9 | Confirmed by 1HNMR analysis |
| | mol %), K₂CO₃ (5 eq), THF, H₂O, rt, 16 h. | |
| 2 | cpd-3 (6.0 g, 1eq.), cpd-4 (3.55 g, 1eq.), | Purification under progress. |
| | L-proline (1.0 eq.), EtOH, reflux, 12 h | Compound was purified by column |
| | | chromatography on silica gel, |
| | | followed by second purification |
| | | with ether trituration. |
| | | Isolated cpd-5: 3.1 g (Y: 37%) |
| | | Confirmed by 1HNMR analysis |
| 3 | cpd-5 (2.5 g, 1eq.), NaOH (1.1 eq), | Purified by trituration with ether |
| | EtOH, H₂O, rt, 3 h | Isolated cpd-6: 1.0 g (Y: 44.2%) |
| | | Confirmed by 1HNMR analysis |
| | | Yield was little low, probably lost |
| | | some acid in the aqueous layer. |

Scheme 2 also shows a synthetic route for compounds 7a and 7b.

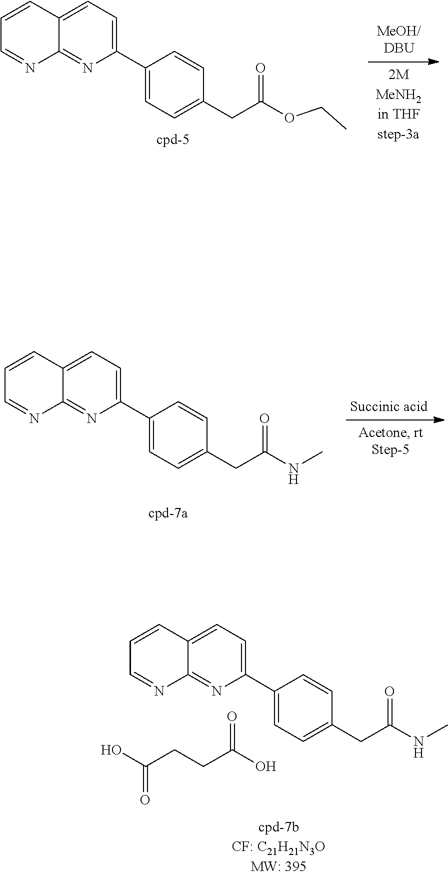

Scheme 2 correlates to the following steps:

| Step | Reaction scale/conditions | Results |
|---|---|---|
| 3a | cpd-5 (0.3 g, 1eq.), MeNH₂ (2M in THF, | Crude compound was purified by |
| | 5.5 mL, 10 eq.), DBU (0.2 eq.), rt, 3 | trituration with Ether. |
| | days. | Isolated cpd-7a free base: 0.28 g |
| | | (Y: quant.). |
| | | Confirmed by 1HNMR and 13C |
| | | NMR and LCMS analyses. |
| 3a | cpd-5 (1.5 g, 1eq.), MeNH₂ (2M in THF, | Crude compound was purified by |
| | 12.8 mL, 5 eq.), DBU (0.2 eq.), rt, 3 | trituration with Ether. |
| | days. | Isolated cpd-7a free base: 1.1 g (Y: |
| | | 77%). |
| | | Confirmed by 1HNMR and 13C |
| | | NMR and LCMS analyses. |
| | | HPLC purity: >98% |
| 3a | cpd-5 (1.6 g, 1eq.), MeNH₂ (2M in THF, | Crude compound was purified by |
| | 12.8 mL, 5 eq.), DBU (0.2 eq.), rt, 3 | trituration with Ether followed by |
| | days. | EtOAc. |
| | | Isolated cpd-7a free base: 1.28 g |
| | | (Y: 84.4%). |
| | | Confirmed by 1HNMR and 13C |
| | | NMR and LCMS analyses. |
| 5 | cpd-7a (1.0 g, 1eq.), Succinic acid | Crude compound was purified by |
| | (0..22 g, 0.5eq.), Acetone (10 mL), 60° C., | trituration DCM. |
| | 16 h. | Isolated cpd-7b: 0.8 g (56.2%) |
| | | Compound was confirmed by |
| | | 1HNMR spectroscopy. |

Scheme 3 shows a synthetic route for an intermediate of 4C.

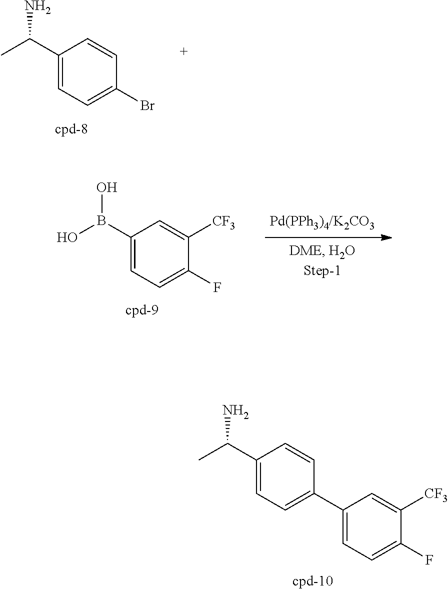

Scheme 4 shows a synthetic route for 4C free base.

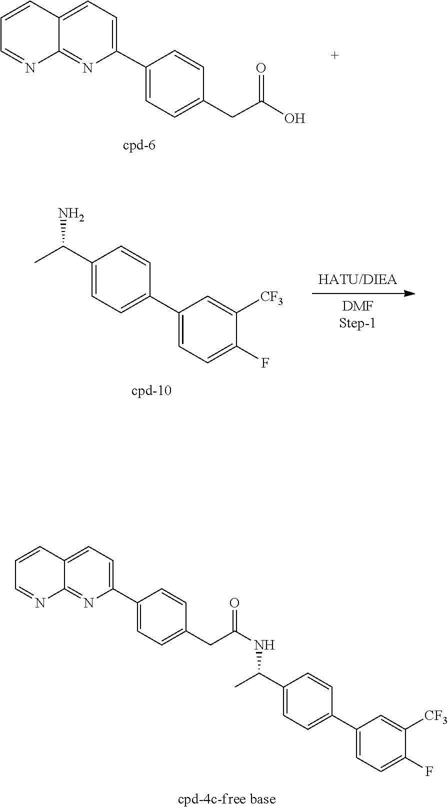 [SMILES: O=C(O)Cc1ccc(-c2ccc3cccnc3n2)cc1 ; C[C@H](N)c1ccc(-c2ccc(F)c(C(F)(F)F)c2)cc1 ; C[C@H](NC(=O)Cc1ccc(-c2ccc3cccnc3n2)cc1)c1ccc(-c2ccc(F)c(C(F)(F)F)c2)cc1]

Scheme 5 shows an alternative synthetic route for 4C free base.

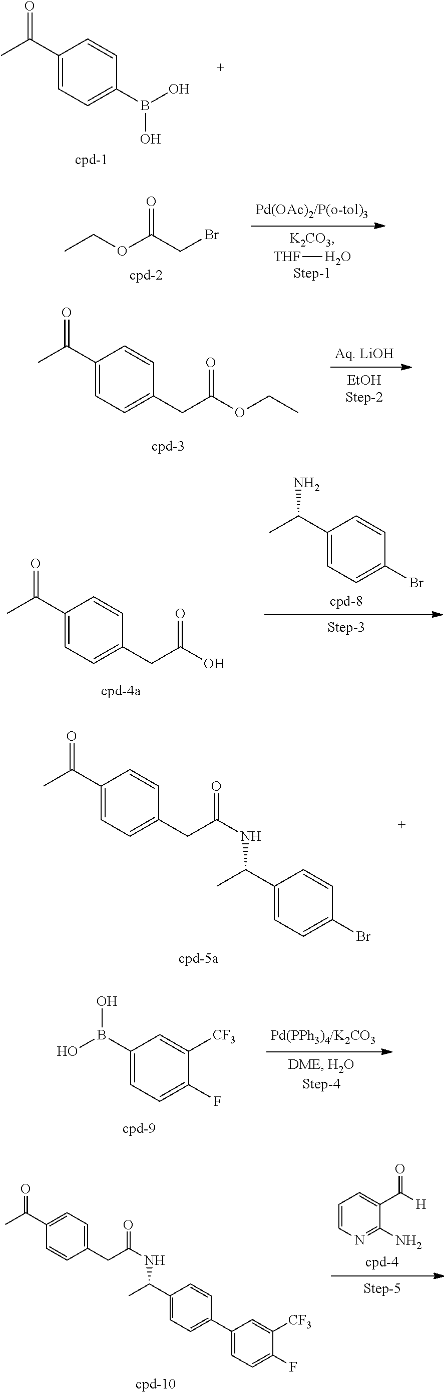

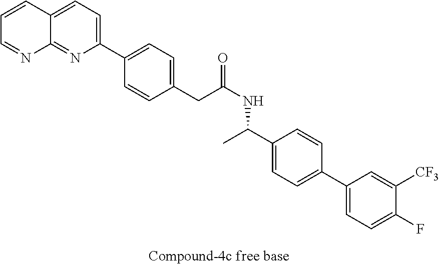 [SMILES: C[C@H](NC(=O)Cc1ccc(-c2ccc3cccnc3n2)cc1)c1ccc(-c2ccc(F)c(C(F)(F)F)c2)cc1]

Scheme 5 correlates to the following steps:

| Step | Reaction scale/conditions | Results |
|---|---|---|
| 2 | cpd-3 (1.0 g, 1eq.), LiOH•H₂O (0.61 g, | Isolated cpd-4a: 0.7 g (Y: 94%) |
| | 3.0 eq), H₂O (3 mL), EtOH, rt, 3 h | Confirmed by 1HNMR analysis |
| 3 | cpd-4a (0.8 g, 1eq.), cpd-8 (0.7 mL, | Isolated cpd-5: 0.3 g (Y: 19%) |
| | 1.1eq.), EDC•HCl (1.29 g, 1.5 eq.), DIEA | Confirmed by 1HNMR analysis |
| | (1.6 mL, 1.0 eq.), DCM, rt, 16 h | |
| 4 | cpd-5a (0.25 g, 1eq.), cpd-9 (0.29 g, 2.0 | Crude was purified by column |
| | eq.), Pd(PPh₃)₄ (5 mol %), K₂CO₃ (1.5 | chromatography on silica gel |
| | eq.), ACN, 80° C., 16 h | Isolated cpd-10: 0.21 g (Y: 69%) |
| | | Confirmed by 1HNMR analysis |
| 5 | cpd-10 (0.2 g, 1eq.), cpd-4 (55.1 mg, | Isolated cpd-4C free base: 0.2 g |
| | 1eq.), L-proline (1.0 eq.), EtOH, reflux, | (Y: 84%) |
| | 24 h. | Confirmed by 1HNMR analysis |

### Example 2

The present example provides uses and efficacy of a composition (e.g., gel) of the present disclosure.

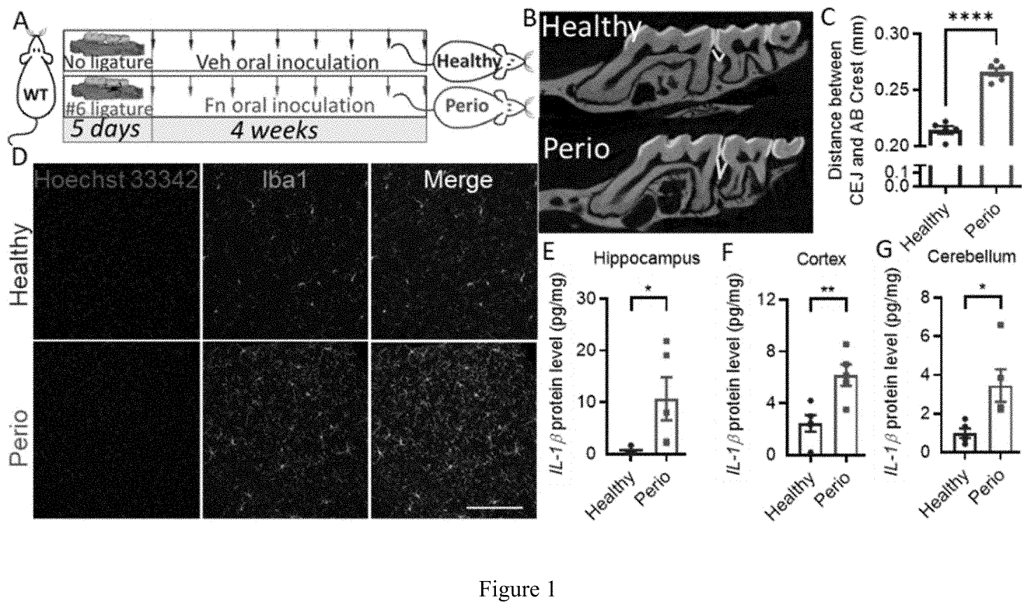

A novel mechanism connecting chronic peripheral inflammation and neuroinflammation. Using chronic periodontitis as a peripheral inflammation model, it was shown that local inflammation in oral cavity significantly increase neural inflammation. Inflammation was induced in 14-week-old C57/B6 mice by placing a ligature on the 2nd maxillary molar for 5-days plus twice/week inoculations of a key periodontal pathogen Fusobacteria nucleatum (Fn) for 4-weeks (FIG. 1A). Mice exhibited a significant alveolar bone loss suggesting local inflammation (FIG. 1B-C). This local inflammation was accompanied with the induction of neuroinflammation evidenced by the activation of microglia (FIG. 1D) and increased IL1β levels in hippocampus, cortex and cerebellum (FIG. 1E-G).

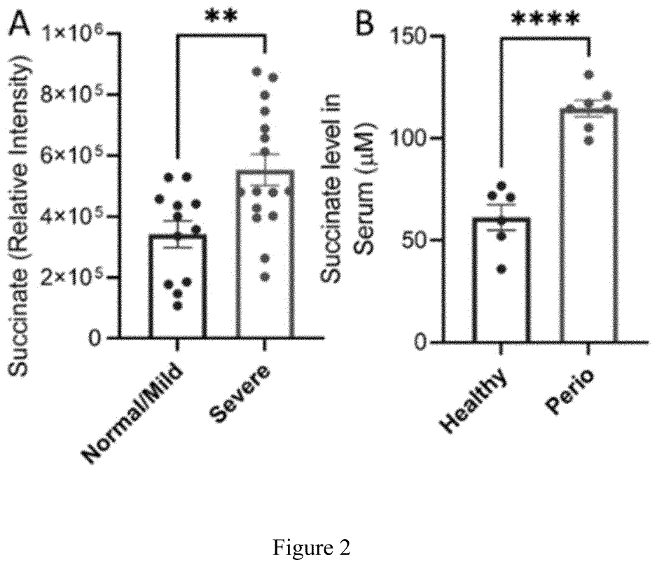

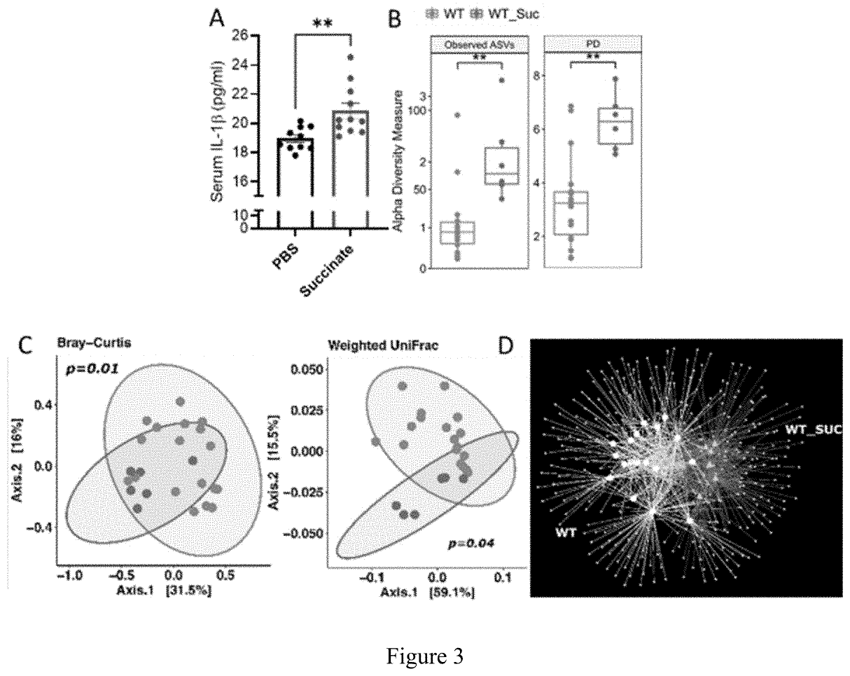

Succinate role in peripheral inflammation. Elevated succinate can cause inflammation in gingival tissues. Succinate elevation was found in the gingival crevicular fluid of periodontal patients, and we observed a significant increase of succinate in the subgingival plaques of patients with severe periodontitis (FIG. 2A). Importantly, the elevation of succinate was recapitulated in our periodontitis mouse model (FIG. 2B). Mimicking succinate elevation by daily administrations of succinate for 4 weeks increased serum IL-1β levels (FIG. 3A) and dysbiosis in the oral microbiota demonstrated by alpha and beta diversity analysis (FIG. 3B-C). The network plot indicated a clear partition between succinate and PBS treated groups (FIG. 3D).

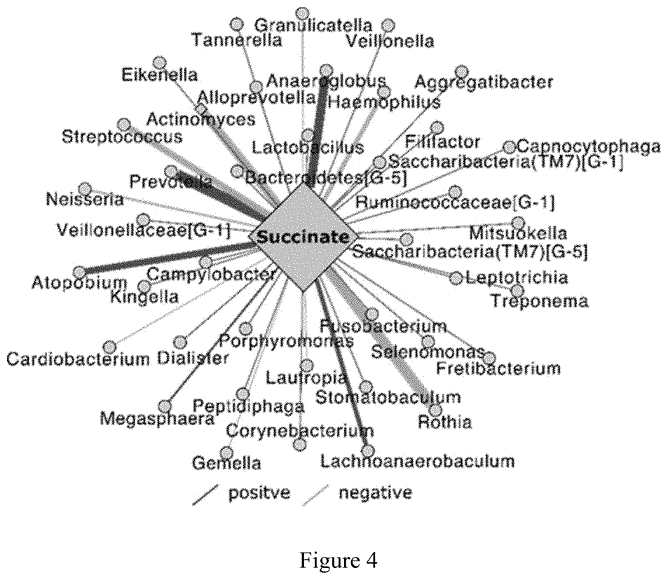

To determine the correlation of higher succinate and oral microbiome data, Spearman's rank correlation coefficient for comparing the relative abundance of the top 40 genus and succinate level in samples using the Hmisc r package (v.4.5.0) (FIG. 4) was used. Correlation-based network plot generated in MetScape 3 for Cytoscape (v. 3.8.2) indicated that many pathogens such as Prevotella, Porphyromonas, Atopobium, Filifactor, Tannerella, Anaeroglobus Saccharibacteria Treponema, Leptotrichia and Fusobacterium were positively correlated with elevated succinate (red) (FIG. 4), which further proved that succinate elevation positively correlated with the abundance of GI pathogens.

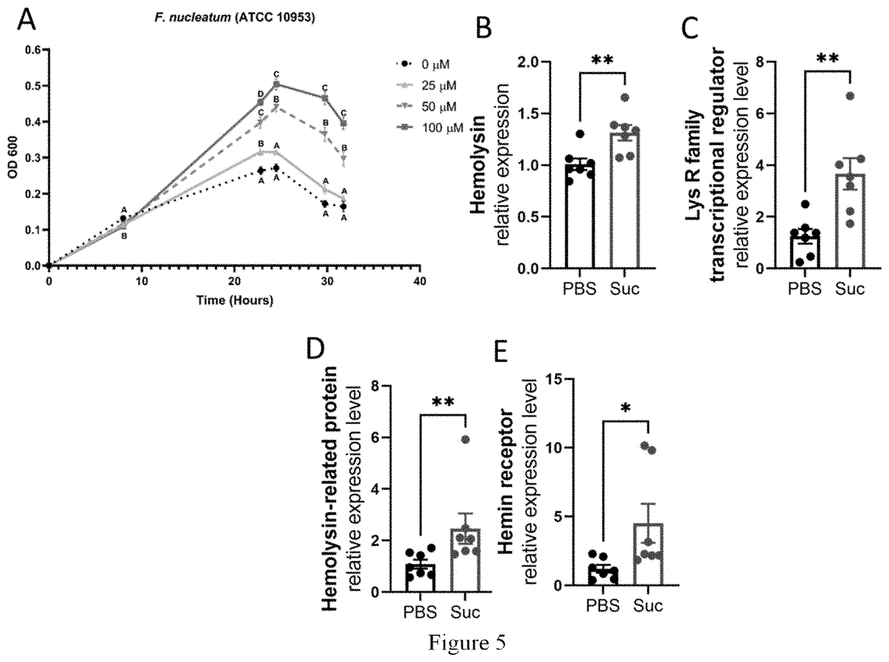

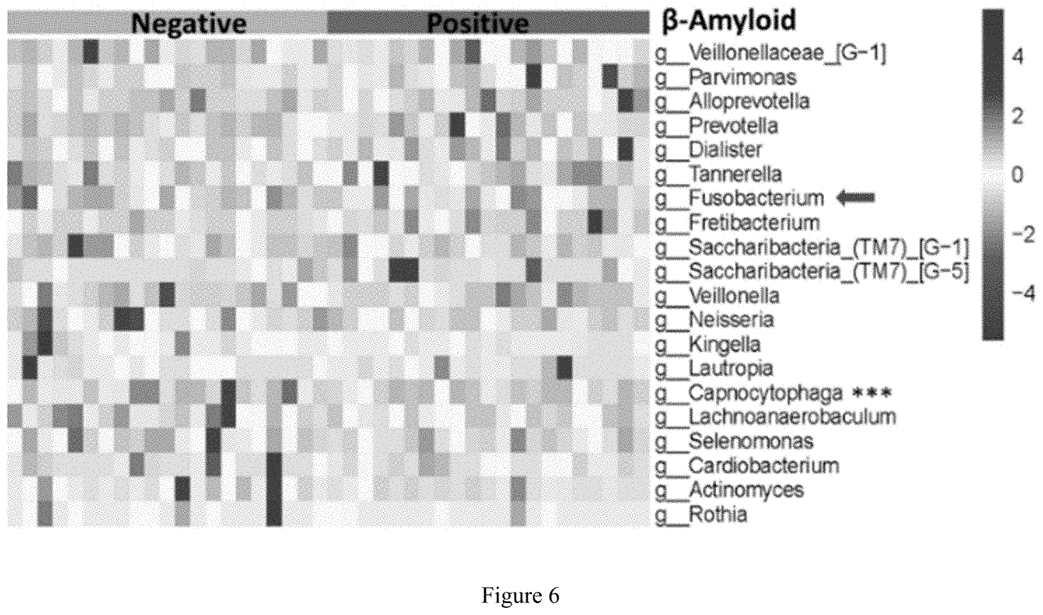

Succinate promotes growth of GI Pathogens. GI tract bacteria such as F. nucleatum, an anchoring pathogen which can instruct biofilm formation to induce inflammation was elevated when grown in the presence of succinate (FIG. 5A). The expression of virulence factors such as Hemin receptor, Hemolysin, Hemolysin-related protein, and Lys R Family transcriptional regulator, were elevated by succinate (FIG. 5B-E). These results suggested that elevated succinate can influence the GI microbiome and modulates the virulence factors of GI bacteria. F. nucleatum is a prominent pathogen which plays a significant role in biofilm formation and supports the growth of other GI pathogens also appeared with significant higher abundance in the subgingival plaques of subjects with positive versus negative detection of Amyloid beta (Aβ) in CSF (FIG. 6).

Provided herein is data supporting a mechanism that provides a causative connection between chronic oral inflammation such as periodontitis and neuroinflammation. Through a succinate/SUCNR1-dependent mechanism, chronic oral inflammation induced succinate accumulation triggers oral dysbiosis and activates SUCNR1 to increase IL1β at systemic and tissue levels, which collectively enhances neuroinflammation.

Aging creates endo-luminal bacterial dysbiosis and succinate elevation impact gut microbiome and host immune responses.

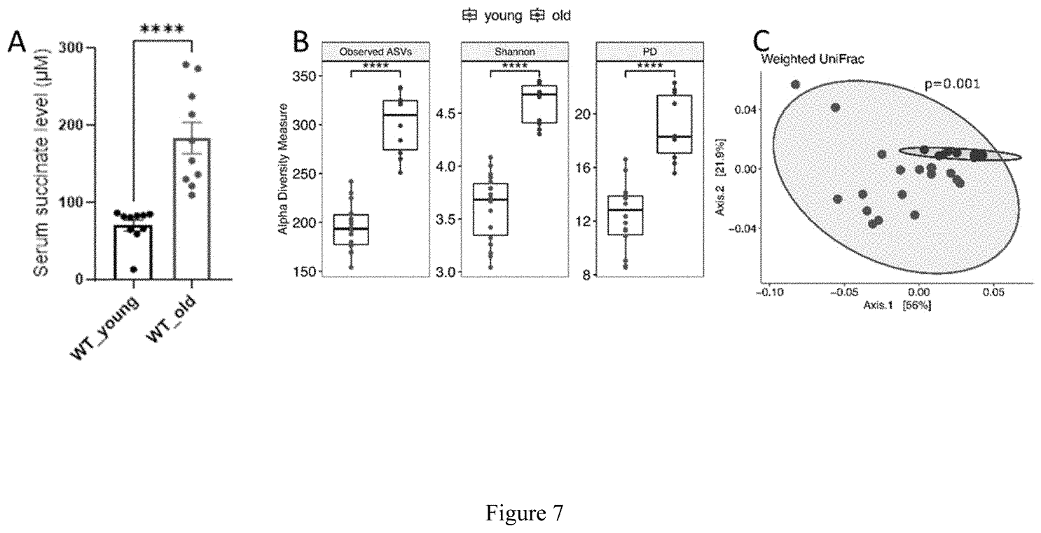

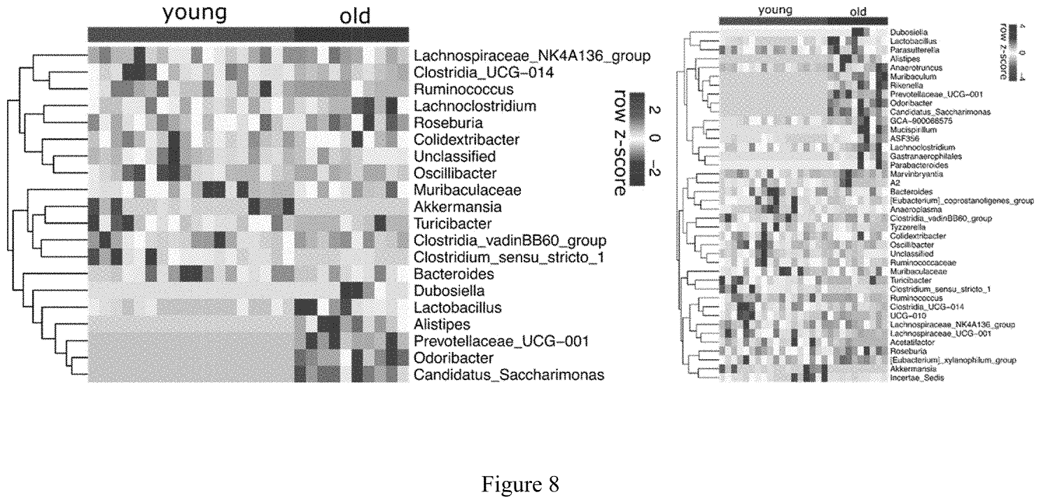

In order to elucidate whether there is elevation of succinate and alternation of the gut microbiome in young and old mice, we compared the gut microbiota of young (4-month old mice) with old mice (24-month old mice) using 16S rRNA sequencing and measured succinate using calorimetric methods. The succinate in the serum of old mice was twofold higher as compared to young mice (7A). Alpha diversity as indicated by observed Amplicon Sequence Variants (ASVs), Shannon, and Faith's phylogenetic diversity (PD) suggested significant differences between young and old mice (FIG. 7B). Principal coordinate analysis indicated that globally bacterial communities in the young and old mice cohorts formed two separate clusters indicative of colonization of old mice with a microbiomes that differed with aging (FIG. 7C). Marked increases were observed at the phylum level in the prevalence of Bacteroidetes and Proteobacteria in the old mice cohort (FIG. 8). Significant increases were also detected at the genus level of Prevotella, Alistipes, candidatus Saccharibacteria (TM7), Lachnoclostridium, and Odoribacter in the old compared to young cohort. Moreover, there was a significant reduction in beneficial bacteria such as Ruminococcus, Oscillobacter, and Akkermansia genera in old mice compared to young mice. Collectively, these data suggest several events: (i) gut bacteria changed with aging; (ii) the intestinal microbiotic profile of old mice is distinct from the microbiotic profile of young mice; and (iii) Prevotella, Alistipes, and Lachnoclostridium, bacterial species associated with succinate fermentation are enriched in old mice compared to young. These data suggested that older mice has higher systemic succinate levels and dysbiotic GI microbiome as compared to young mice.

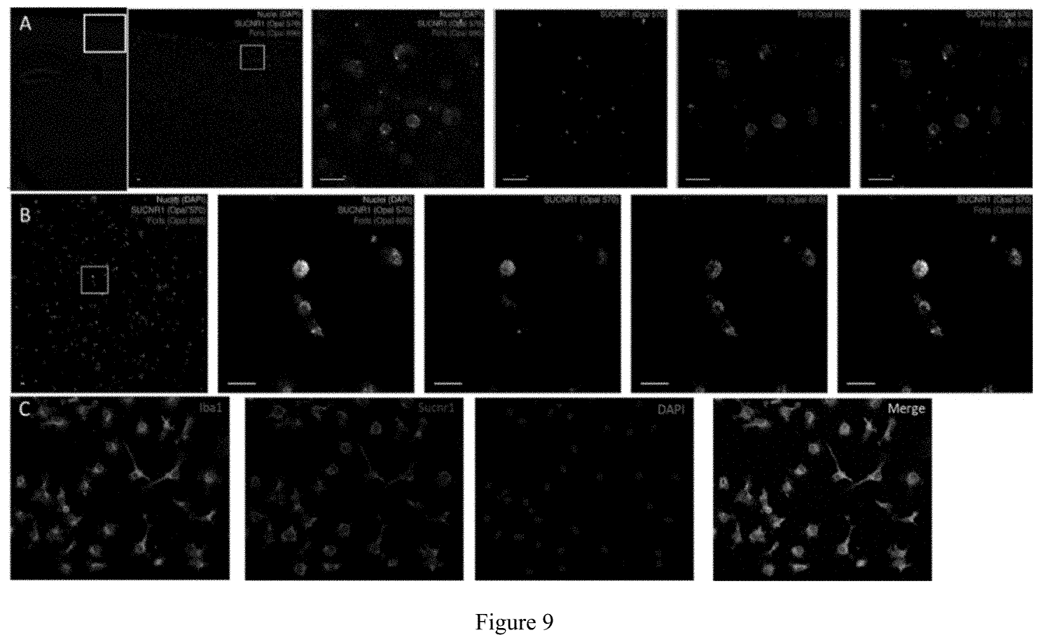

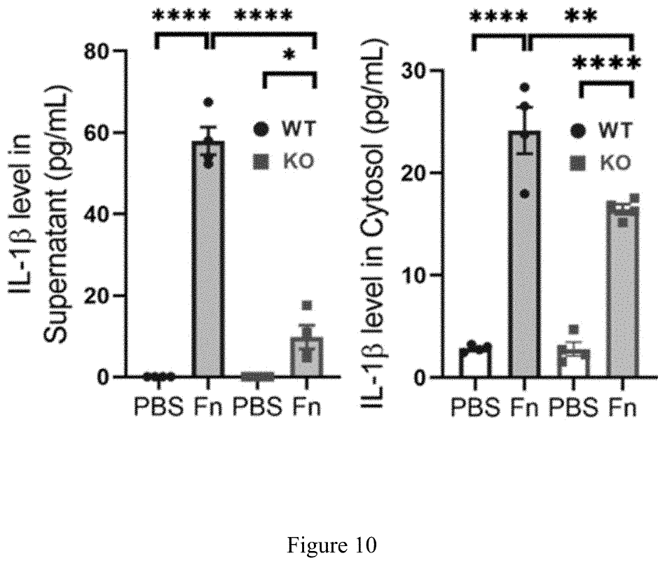

SUCNR1 as a novel target for neuroinflammation. Microglia are crucial for the homeostasis within the brain. This finding is the first to show the presence of SUCNR1 in microglia (FIG. 9) and its effect on microglial function (FIG. 10). It was noted that succinate may have multifaceted actions and initiate various signaling based on its cellular locations. The role of SUCNR1 mediated function in microglia has not been reported. Addressed herein is the function of microglial SUCNR1 mediated neuroinflammation. This provided evidence supporting SUCNR1 as a novel target for neuroinflammation prevention and treatment.

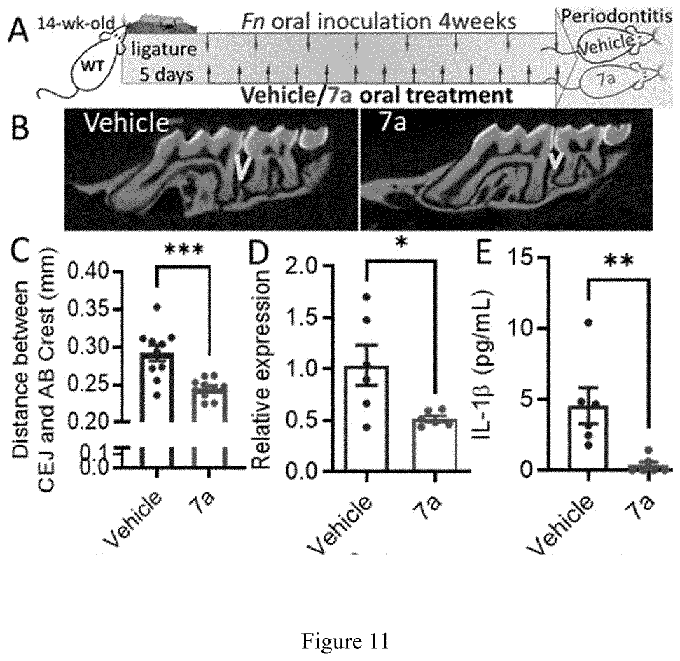

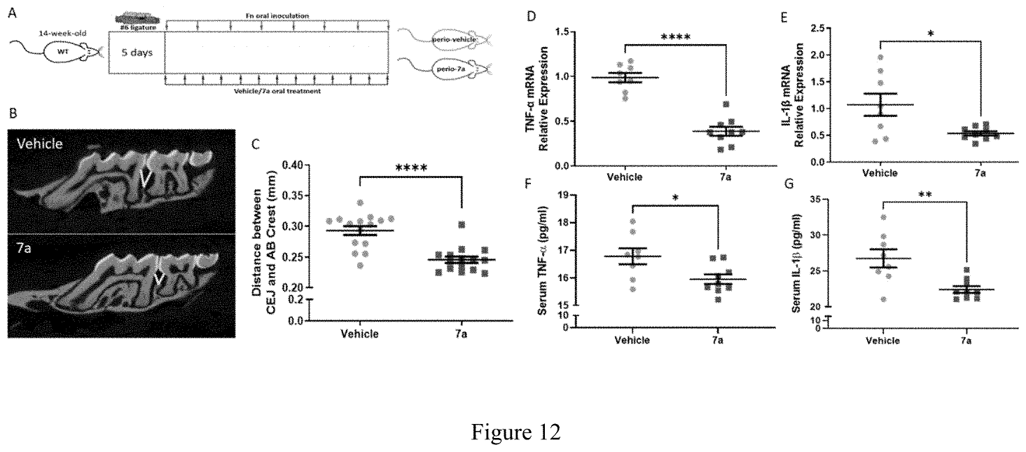

Oral treatment of SUCNR1 antagonist reduce local, systemic and neuroinflammation. Treating periodontitis alleviates neuroinflammation. Provided herein is an oral topical gel-formulation with a specific SUCNR1 antagonist 7a, which inhibited periodontitis (FIG. 11A-C). As expected, 7a reduced the local IL1β levels in the gingiva (FIG. 11D) and systemic levels in the serum (FIG. 12E). Interestingly, significant reduction of IL1β levels was also observed in the hippocampal and cerebellum tissues (FIG. 11F, H). SUCNR1 activation plays an important role in the oral cavity to induce periodontal related inflammation.

Provided is the role of GI-brain axis in neuroinflammation. These data support that local or systemic succinate elevation can induce inflammation causes GI dysbiosis. Directly regulates the growth of a key gastrointestinal tract (oral and gut) pathogen growth and alter virulence factors. This peripheral inflammation and succinate signaling in neural cells neuroinflammatory responses.

### Example 3

The present example provides uses and efficacy of a composition (e.g., gel) of the present disclosure.

For a formulations study, the polymers poly lactic-co-glycolic acid (PLGA) at 0.26 to 0.54 dL/g, PLGA (0.55-0.75 dL/g in hexafluoroisopropanol [HFIP]), Poly (D,L-lactide)-ester terminated (0.55-0.75 dL/g in CHCl₃), poly(ε-caprolactone)-ester terminated, (0.65-0.85 dL/g in CHC13) were purchased from Lactel® Absorbable Polymers. The specified amount of polymer was dissolved in 4 mL of a solvent system. Compound-7a was added to the polymeric solution (final concentration of 5.0 mg/mL) and mixed by vortexing until complete dissolution. Concentrations of the obtained formulations were then assessed by UV-Vis analysis.

Six in situ gel formulations (ISGs) were produced by varying polymer concentration, changing the hydrophobicity of the solvent system, and use of different plasticizers (such as PEG200). All these ISGs were analyzed by FT-IR/ATR and HPLC assay for drug-polymer compatibility. In vitro drug release experiments were performed to assess the profile of sustained release with controlled initial burst release. One gel formulation was chosen to treat periodontitis animals in-vivo at 1 μg/10 g body weight three times/week (FIG. 11).

PRM001: 20% w/v, PLGA (0.26-0.54 dL/g) was dissolved in a mixture of BB/NMP (7:3) by heating at 55° C. for 4 h. Appropriate quantity of cpd 7a was added to this polymer solution, to make up 5 mg/mL concentration. The mixture of heated at 45° C. for ˜2 h to get the desired PRM001 ISG formulation (a clear solution)

PRM002: 10% w/v, PLGA (0.26-0.54 dL/g) was dissolved in a mixture of BB/NMP (1:1) by heating at 55° C. for 6 h. Appropriate quantity of cpd 7a was added to this polymer solution, to make up 5 mg/mL concentration. The mixture of heated at 45° C. for ˜2 h to get the desired PRM002 ISG formulation (a clear solution)

PRM003: 25% w/v, PLGA (0.26-0.54 dL/g) was dissolved in a mixture of BB/NMP (1:4) and 3.5% PEG200, by heating at 55° C. for 4 h. Appropriate quantity of cpd 7a was added to this polymer solution, to make up 5 m g/mL concentration. The mixture of heated at 45° C. for ˜2 h to get the desired PRM003 ISG formulation (a clear solution)

PRM004: 10% w/v, poly(D,L-lactide)-ester terminated (0.55-0.75 dL/g in CHCl₃) was dissolved in a mixture of BB/NMP (1:1) by heating at 55° C. for 24 h.

Appropriate quantity of cpd 7a was added to this polymer solution, to make up 5 mg/mL concentration. The mixture of heated at 45° C. for ˜24 h and the desired PRM004 ISG formulation was obtained (clear solution).

PRM005: 10% w/v, PLGA (0.55-0.75 dL/g in HFIP) was dissolved in a mixture of BB/NMP (1:1) by heating at 55° C. for 24 h. Appropriate quantity of cpd 7a was added to this polymer solution, to make up 5 mg/mL concentration. The mixture of heated at 45° C. for ˜24 h and the desired PRM005 ISG formulation was obtained (clear solution).

PRM006: 10% w/v, poly(ε-caprolactone)-ester terminated (0.65-0.85 dL/g in CHCCl₃) was dissolved in a mixture of BB/NMP (1:1) by heating at 55° C. for 24 h. Appropriate quantity of cpd 7a was added to this polymer solution, to make up 5 mg/mL concentration. The mixture of heated at 45° C. for ˜24 h and the desired PRM002 ISG formulation was obtained (clear solution).

TABLE
Compositions of the cpd-7a ISG's

| Form-code | Polymer vehicle (%) | Solvent** | Concentration* |
|---|---|---|---|
| PRM001 | PLGA (0.26-0.54 dL/g) (20% w/v) | 30% BB in NMP | 5.1 mg/mL |
| PRM002 | PLGA (0.26-0.54 dL/g) (10% w/v) | 50% BB in NMP | 5.0 mg/mL |
| PRM003 | PLGA (0.26-0.54 dL/g) (25% w/v) | 25% BB in NMP | 5.0 mg/ml |
| | and 3.5% PEG200 | | |
| PRM004 | Poly(D,L-lactide)-ester terminated | 50% BB in NMP | 5.2 mg/mL |
| | (0.55-0.75 dL/g in CHCl₃), (10% | | |
| | w/v) | | |
| PRM005 | PLGA (0.55-0.75 dL/g in HFIP) | 50% BB in NMP | 5.1 mg/mL |
| | (10% w/v) | | |
| PRM006 | poly(ε-caprolactone)-ester | 50% BB in NMP | Observed precipitate |
| | terminated, (0.65-0.85 dL/g in | | formation after |
| | CHCl₃), (10% w/v) | | storage. |
| PRM007 | PLGA (0.55-0.75 dL/g in HFIP) | 25% BB in NMP | 4.0 mg/ml |
| | (25% w/v) and 3.5% PEG200 | | |

*Concentration was estimated using HPLC assay.
**BB: benzyl benzoate; NMP: N-methyl-2-pyrrolidone; PEG: polyethylene glycol 200

Analysis of ISG's: All six ISG's were analyzed by FT-IR/ATR and HPLC assay for drug-polymer compatibility.

PRM002: scaled up the PRM002 formulation (with cpd-7a, 50 mg) of 5 mg/mL concentration (using the protocol described above). This batch of formulation is being used for in vivo experiments.

The ISG was analyzed by HPLC assay.

To determine if succinate receptor 1 inhibitors could reduce inflammation, succinate receptor 1 inhibitors were administered to mice. In this study (FIG. 11), mice with periodontal disease were used because of the resulting neuroinflammation from the periodontal disease. Topical application of POC7a gel formulation was administered at 1 μg/10 g body weight three times/week. This reduced inflammation and reduced alveolar bone loss associated with ligature-induced periodontal disease (FIG. 11).

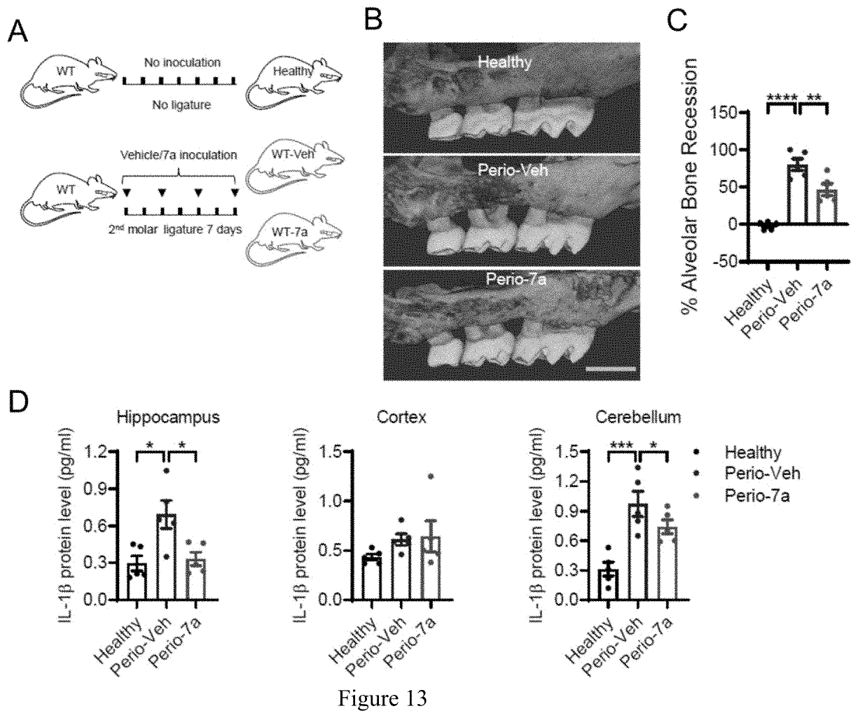

Treatment duration at 1 μg/10 g body weight once/week for 10 weeks in mice with periodontitis (FIG. 12) and daily for 7 days (FIG. 13) has been tested. Both showed reduction of inflammation (including neuroinflammation) and bone loss. These results suggested that both once per week treatment for weeks or daily treatment for days can reduce inflammation, neuroinflammation, as well as periodontal bone loss in mice. As the dosing was based on the body weight, it is anticipated similar dosing and regimes from daily to weekly will work in human. Accordingly, application of the gel formulation to periodontal tissue can used to reduce inflammation (including neuroinflammation).

### Example 4

The present example provides uses of a composition (e.g., for systemic administration) of the present disclosure.

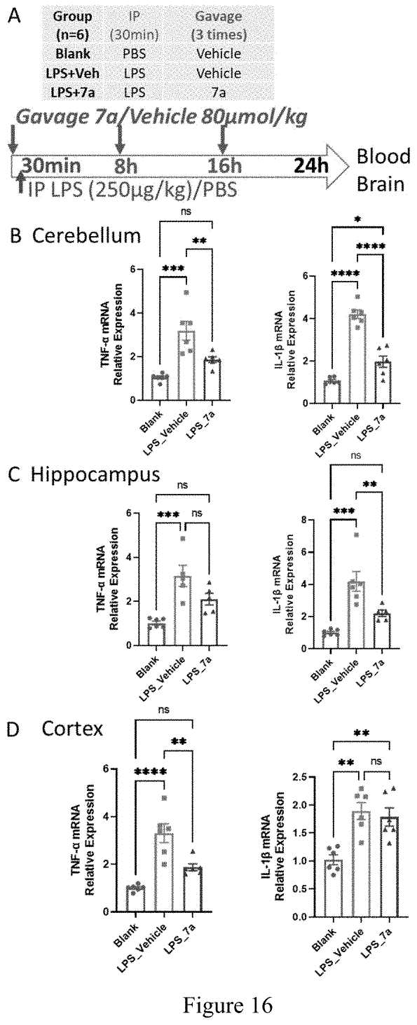

Targeting SUCNR1 with topical 7a application reduced periodontitis as well as the associated neuroinflammation in mice. It is important to know whether systemic administration of 7a directly reduced neuroinflammation. Thus, mice were pretreated 7a or vehicle through oral gavage 30 minutes before giving LPS intraperitoneally to induce neuroinflammation. The administration of 7a at 8 hours and 16 hours was repeated after the first dose. The mouse blood samples were collected through cardiac puncture at 24 hours (FIG. 16A). Immediately afterward, PBS was used to perfuse the mice to minimize the impact of blood cells to the gene expression of the brain tissues. The brain tissue samples were collected and the cerebellum, hippocampus, and cortex were micro-dissected and processed for RNA extraction and gene expression analysis. LPS significantly induced the expression of pro-inflammatory cytokines TNFα and IL1β in all brain regions tested (significant changes in LPS_Vehicle vs Blank, FIG. 16B-D). Importantly, in mice treated with LPS, the group received 7a by oral gavage (LPS_7 a) exhibited less induction of TNFα and IL1β expression in the cerebellum and hippocampus regions (FIG. 16B-C). In the cortex, TNFα expression was also reduced by 7a (FIG. 16D). Of note, this result demonstrated that targeting SUCNR1 with oral administrations of its antagonist can significantly reduce neuroinflammation as indicated by the expression levels of pro-inflammatory cytokines.

The 7a solution was prepared as follows. 100 mM 7a stock solution was prepared using DMSO. Then the 7a stock solution was further diluted with PBS to become 20 mM 7a working solution and used for oral gavage to mice. DMSO in PBS (at 20% v/v) was used as vehicle. Based on the animals' body weight, 25 gram mice will receive 100 μL gavage volume) 7a working solution or vehicle. The treatment dose of 7a is 80 μmol/kg.

### Example 5

The present example provides uses of a composition (e.g., gel) of the present disclosure.

Compound 7a synthesis and gel formulation. All reagents/solvents were used as received from commercial suppliers unless otherwise noted. Reactions were conducted in screw-cap glass vials with Teflon-lined caps/round bottom flasks. Reactions were monitored by thin-layer chromatography (TLC) using a TLC silica gel 60 F254 glass plate. Compounds were detected by UV (254 nm) or staining (ninhydrin or phosphomolybdic acid). Column chromatography was performed using 230-400 mesh silica gel in all cases unless otherwise mentioned.

Nuclear magnetic resonance (1H NMR, 13C NMR) experiments were performed on Bruker Avance 400, 500, or 600 MHz NMR spectrometer and are referenced to the solvent resonances. Chemical shifts (6) are reported in parts per million (ppm), and coupling constants (J) were expressed in hertz (Hz). Standard Abbreviations are used to designate resonance multiplicities. For formulations study, the polymers PLGA (0.26-0.54 dL/g), PLGA (0.55-0.75 dL/g in HFIP), Poly (D, L-lactide)-ester terminated (0.55-0.75 dL/g in CHCCl₃), poly (ε-caprolactone)-ester terminated, (0.65-0.85 dL/g in CHCCl₃) were purchased from Lactel Absorbable Polymers. The specified amount of polymer was dissolved in 4 mL of the solvent system. An appropriate quantity of compound-7a (at a final concentration of 5.0 mg/mL) was added to the polymeric solution and mixed by vortexing until complete dissolution.

While the present invention has been described through specific examples, routine modifications to the disclosure will be apparent to those skilled in the art. Such modifications are intended to be within the scope of the disclosure.

1. A method for treating neuroinflammation in an individual, comprising:
administering to the individual a composition comprising a succinate/succinate receptor 1 inhibitor,
wherein the neuroinflammation in the individual is ameliorated or reduced.

2. The method of claim 1, wherein the succinate/succinate receptor 1 inhibitor is administered to a location other than the brain of the individual.

3. The method of claim 1, wherein the composition is administered systemically.

4. The method of claim 1, wherein the neuroinflammation is associated with schizophrenia, autism, depression, a mood disorder, acute disseminated encephalomyelitis, acute optic neuritis, transverse myelitis, neuromyelitis optica, Adrenal Leukodystrophy (ALD), Alcoholism, Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cerebral palsy, Cockayne syndrome, Corticobasal degeneration, Creutzfeldt-Jakob disease, Familial Fatal Insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia, Spinal muscular atrophy, Steele-Richardson-Olszewski disease, Tabes dorsalis, Toxic encephalopathy, LHON (Leber's Hereditary optic neuropathy), MELAS (Mitochondrial Encephalomyopathy; Lactic Acidosis; Stroke), MERRF (Myoclonic Epilepsy; Ragged Red Fibers), PEO (Progressive External Opthalmoplegia), Leigh's Syndrome, MNGIE (Myopathy and external ophthalmoplegia; Neuropathy; Gastro-Intestinal; Encephalopathy), Kearns-Sayre Syndrome (KSS), NARP, Hereditary Spastic Paraparesis, Mitochondrial myopathy, periodontal disease, Friedreich Ataxia, or combinations thereof.

5. The method of claim 1, wherein the succinate/succinate receptor 1 inhibitor is chosen from: 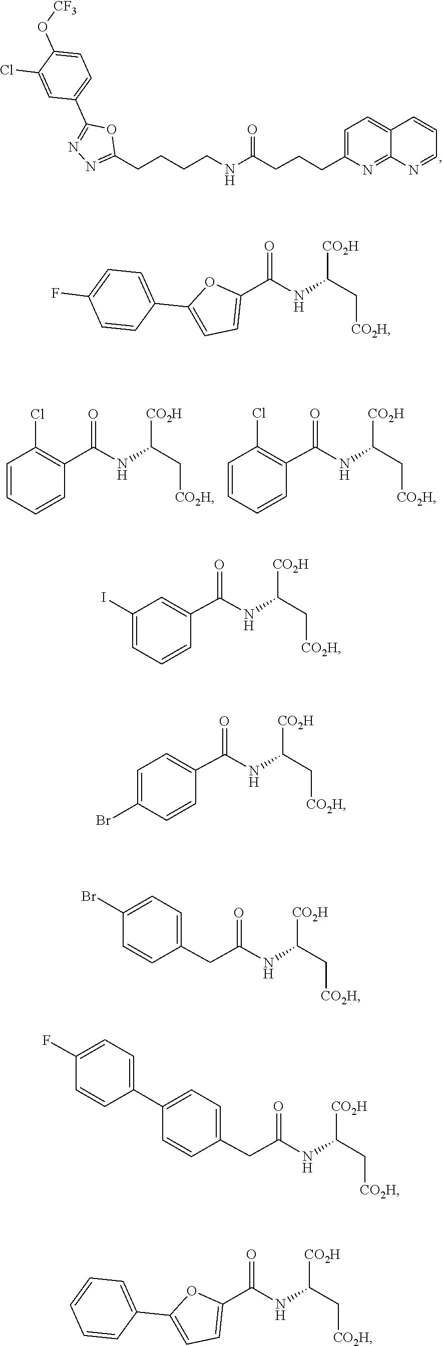 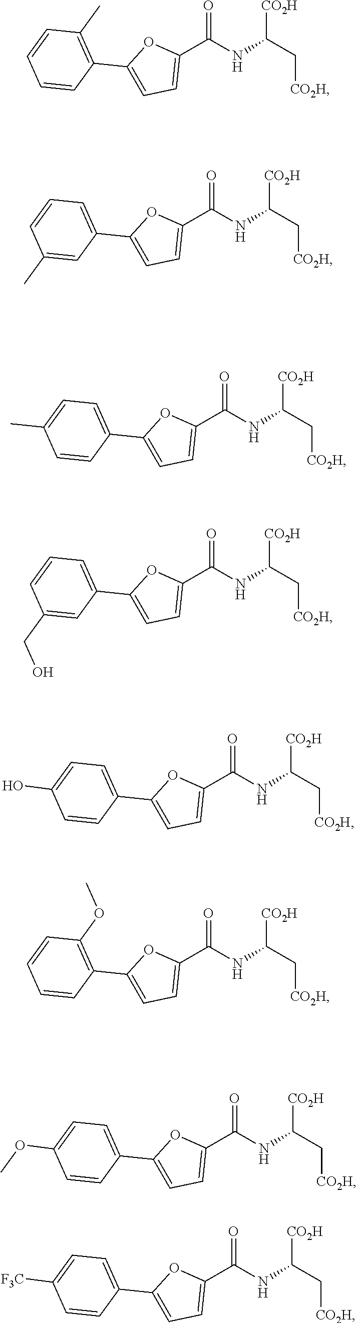 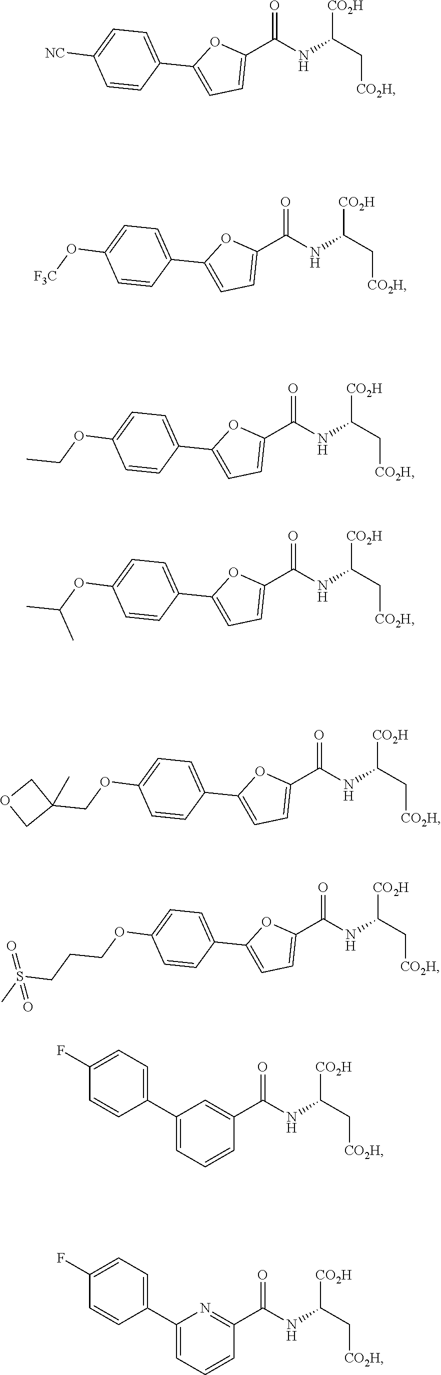 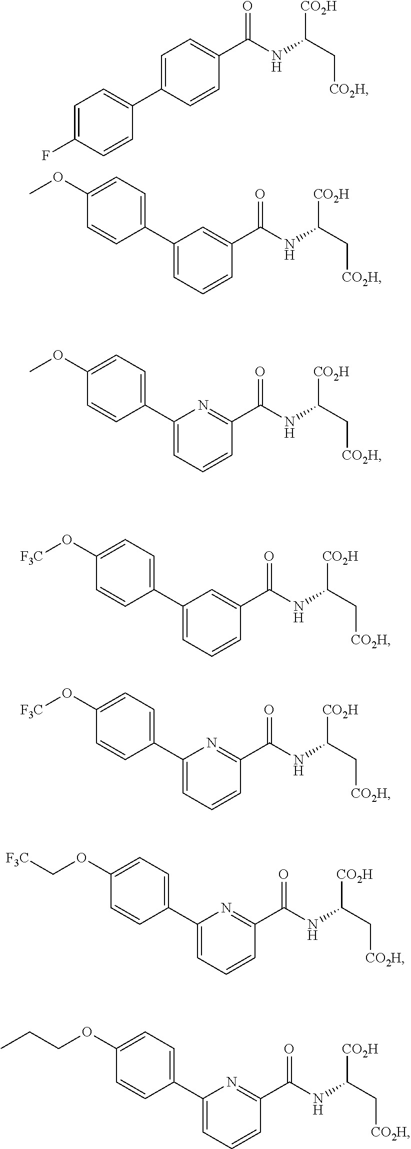 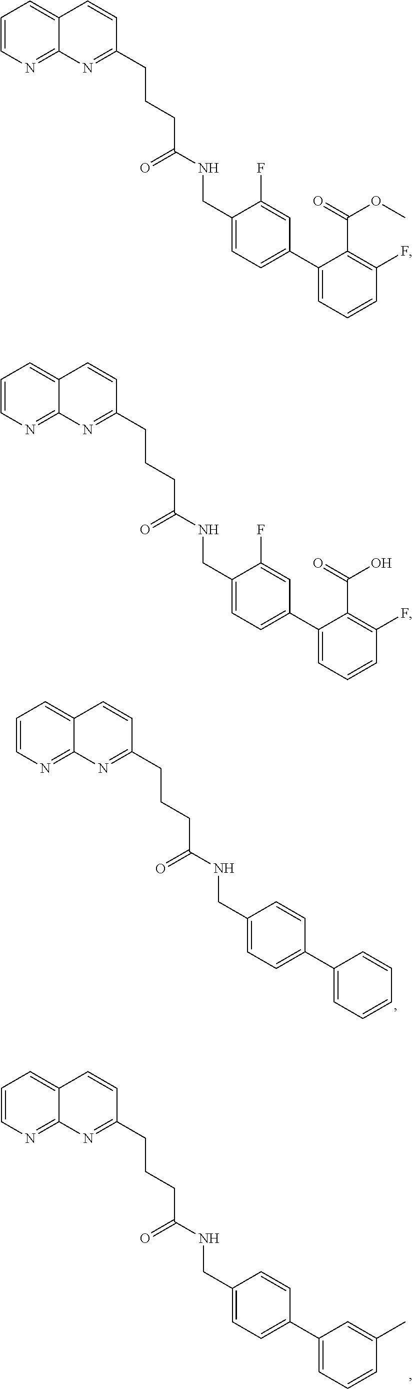 [SMILES: COC(=O)c1c(F)cccc1-c1ccc(CNC(=O)CCCc2ccc3cccnc3n2)c(F)c1 ; O=C(CCCc1ccc2cccnc2n1)NCc1ccc(-c2cccc(F)c2C(=O)O)cc1F ; O=C(CCCc1ccc2cccnc2n1)NCc1ccc(-c2ccccc2)cc1 ; Cc1cccc(-c2ccc(CNC(=O)CCCc3ccc4cccnc4n3)cc2)c1] 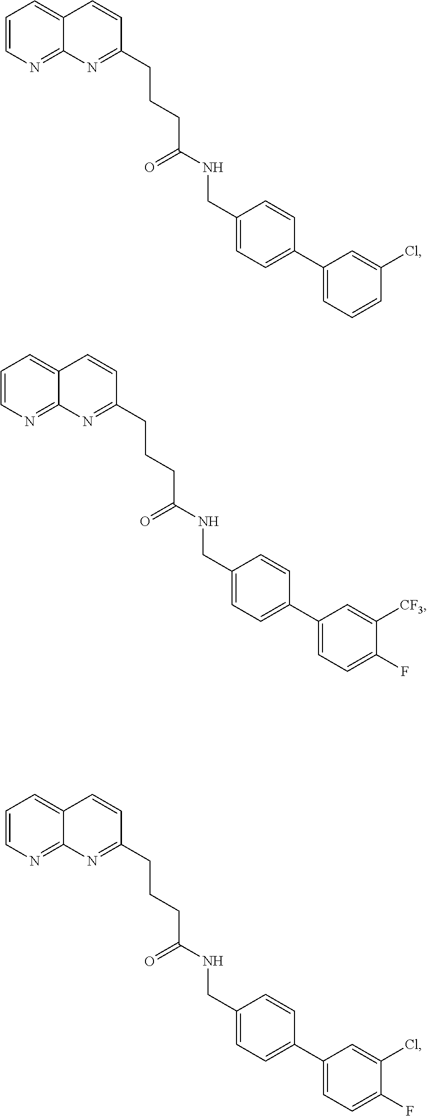 [SMILES: O=C(CCCc1ccc2cccnc2n1)NCc1ccc(-c2cccc(Cl)c2)cc1 ; O=C(CCCc1ccc2cccnc2n1)NCc1ccc(-c2ccc(F)c(C(F)(F)F)c2)cc1 ; O=C(CCCc1ccc2cccnc2n1)NCc1ccc(-c2ccc(F)c(Cl)c2)cc1] 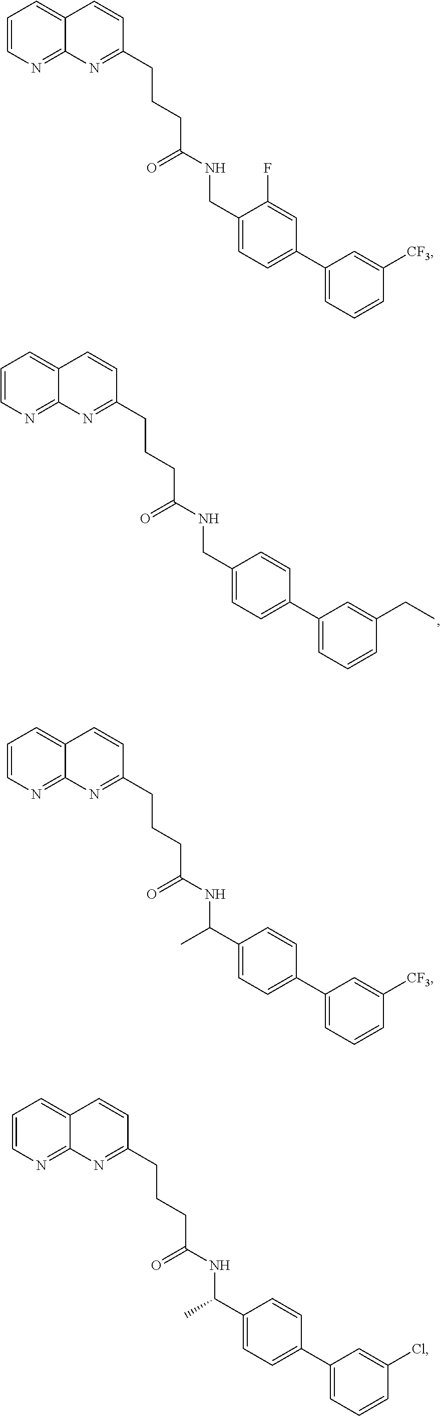 [SMILES: O=C(CCCc1ccc2cccnc2n1)NCc1ccc(-c2cccc(C(F)(F)F)c2)cc1F ; CCc1cccc(-c2ccc(CNC(=O)CCCc3ccc4cccnc4n3)cc2)c1 ; CC(NC(=O)CCCc1ccc2cccnc2n1)c1ccc(-c2cccc(C(F)(F)F)c2)cc1 ; C[C@H](NC(=O)CCCc1ccc2cccnc2n1)c1ccc(-c2cccc(Cl)c2)cc1] 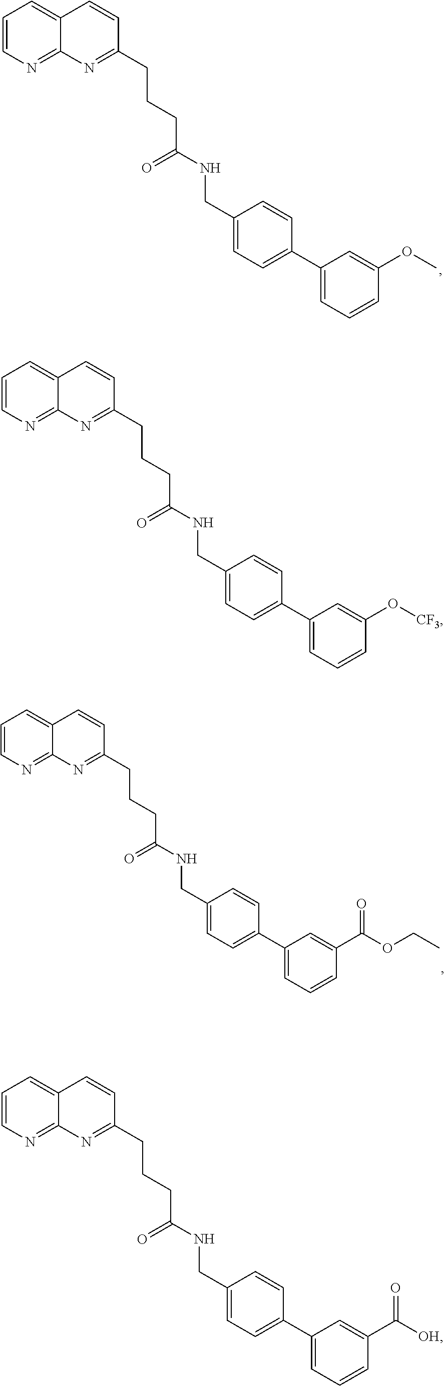 [SMILES: COc1cccc(-c2ccc(CNC(=O)CCCc3ccc4cccnc4n3)cc2)c1 ; O=C(CCCc1ccc2cccnc2n1)NCc1ccc(-c2cccc(OC(F)(F)F)c2)cc1 ; CCOC(=O)c1cccc(-c2ccc(CNC(=O)CCCc3ccc4cccnc4n3)cc2)c1 ; O=C(CCCc1ccc2cccnc2n1)NCc1ccc(-c2cccc(C(=O)O)c2)cc1] 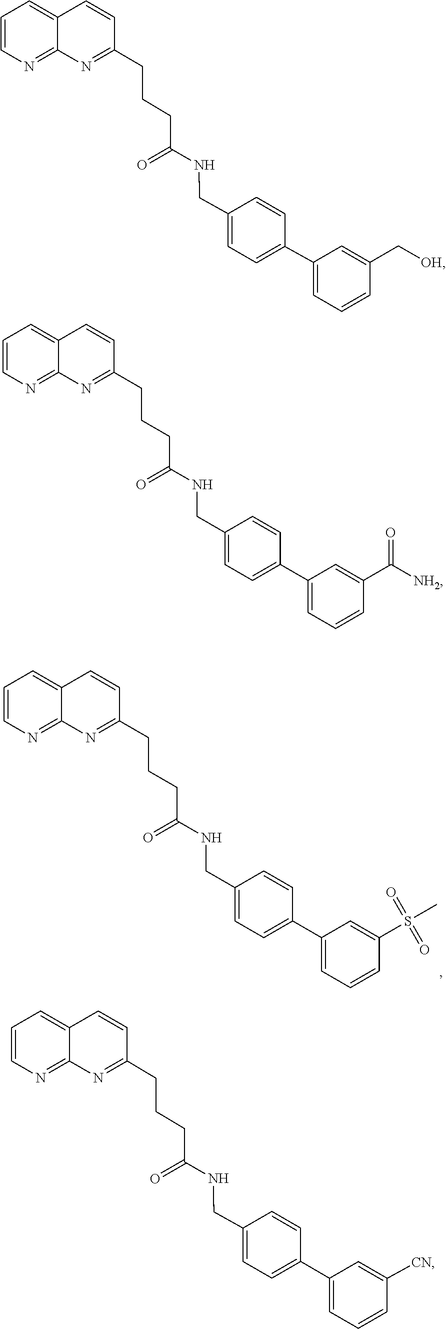 [SMILES: O=C(CCCc1ccc2cccnc2n1)NCc1ccc(-c2cccc(CO)c2)cc1 ; NC(=O)c1cccc(-c2ccc(CNC(=O)CCCc3ccc4cccnc4n3)cc2)c1 ; CS(=O)(=O)c1cccc(-c2ccc(CNC(=O)CCCc3ccc4cccnc4n3)cc2)c1 ; N#Cc1cccc(-c2ccc(CNC(=O)CCCc3ccc4cccnc4n3)cc2)c1] 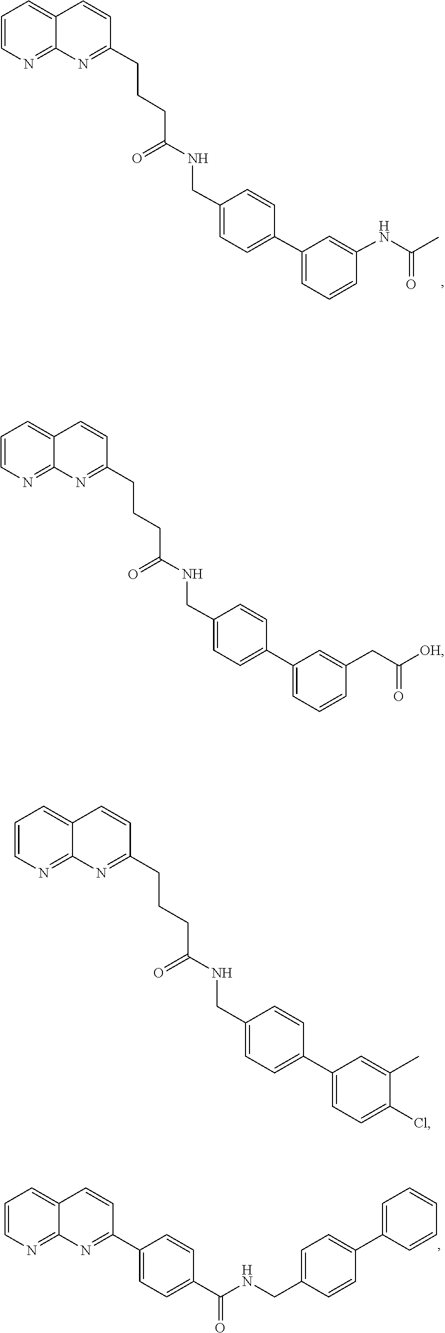 [SMILES: CC(=O)Nc1cccc(-c2ccc(CNC(=O)CCCc3ccc4cccnc4n3)cc2)c1 ; O=C(O)Cc1cccc(-c2ccc(CNC(=O)CCCc3ccc4cccnc4n3)cc2)c1 ; Cc1cc(-c2ccc(CNC(=O)CCCc3ccc4cccnc4n3)cc2)ccc1Cl ; O=C(NCc1ccc(-c2ccccc2)cc1)c1ccc(-c2ccc3cccnc3n2)cc1] 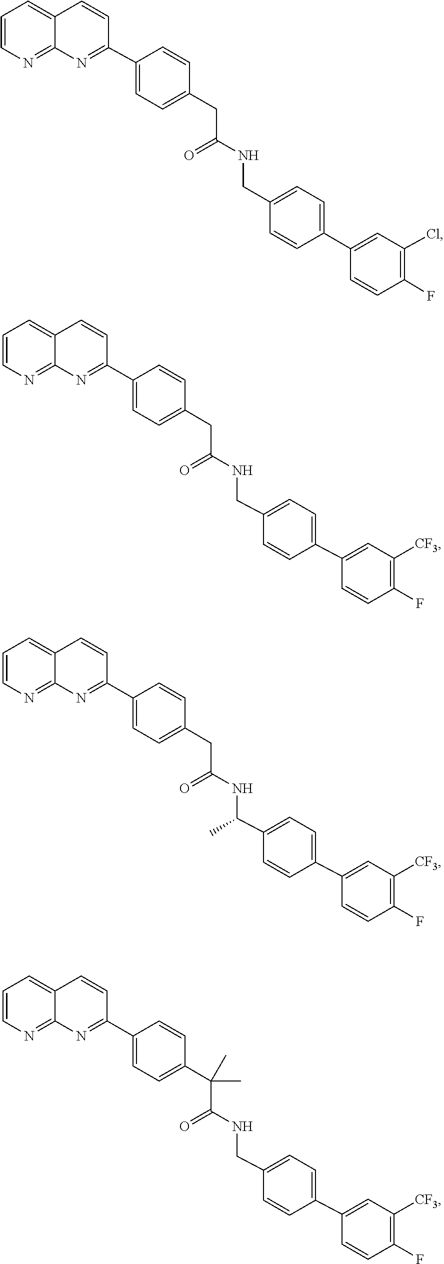 [SMILES: O=C(Cc1ccc(-c2ccc3cccnc3n2)cc1)NCc1ccc(-c2ccc(F)c(Cl)c2)cc1 ; O=C(Cc1ccc(-c2ccc3cccnc3n2)cc1)NCc1ccc(-c2ccc(F)c(C(F)(F)F)c2)cc1 ; C[C@H](NC(=O)Cc1ccc(-c2ccc3cccnc3n2)cc1)c1ccc(-c2ccc(F)c(C(F)(F)F)c2)cc1 ; CC(C)(C(=O)NCc1ccc(-c2ccc(F)c(C(F)(F)F)c2)cc1)c1ccc(-c2ccc3cccnc3n2)cc1] 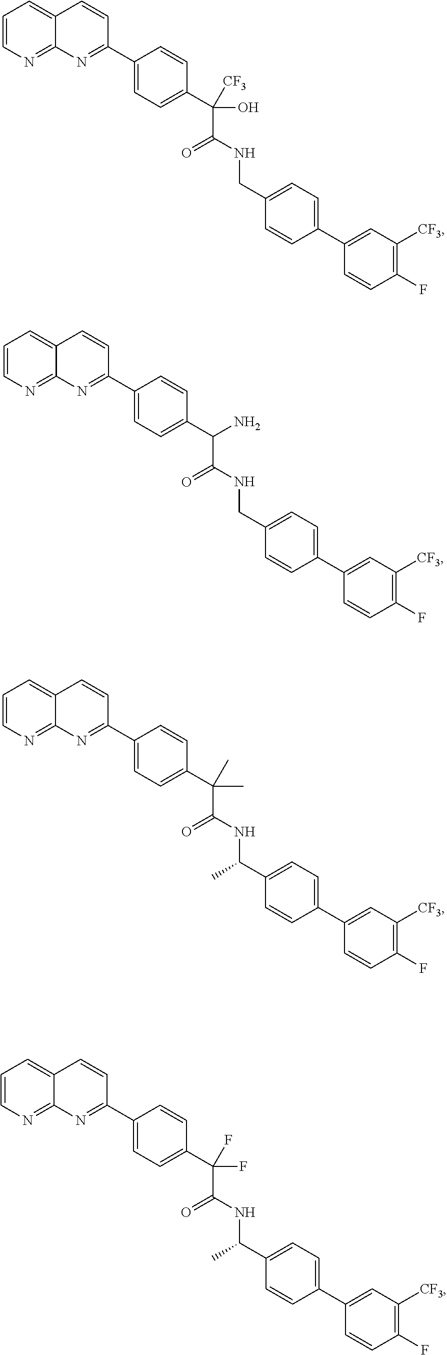 [SMILES: O=C(NCc1ccc(-c2ccc(F)c(C(F)(F)F)c2)cc1)C(O)(c1ccc(-c2ccc3cccnc3n2)cc1)C(F)(F)F ; NC(C(=O)NCc1ccc(-c2ccc(F)c(C(F)(F)F)c2)cc1)c1ccc(-c2ccc3cccnc3n2)cc1 ; C[C@H](NC(=O)C(C)(C)c1ccc(-c2ccc3cccnc3n2)cc1)c1ccc(-c2ccc(F)c(C(F)(F)F)c2)cc1 ; C[C@H](NC(=O)C(F)(F)c1ccc(-c2ccc3cccnc3n2)cc1)c1ccc(-c2ccc(F)c(C(F)(F)F)c2)cc1] 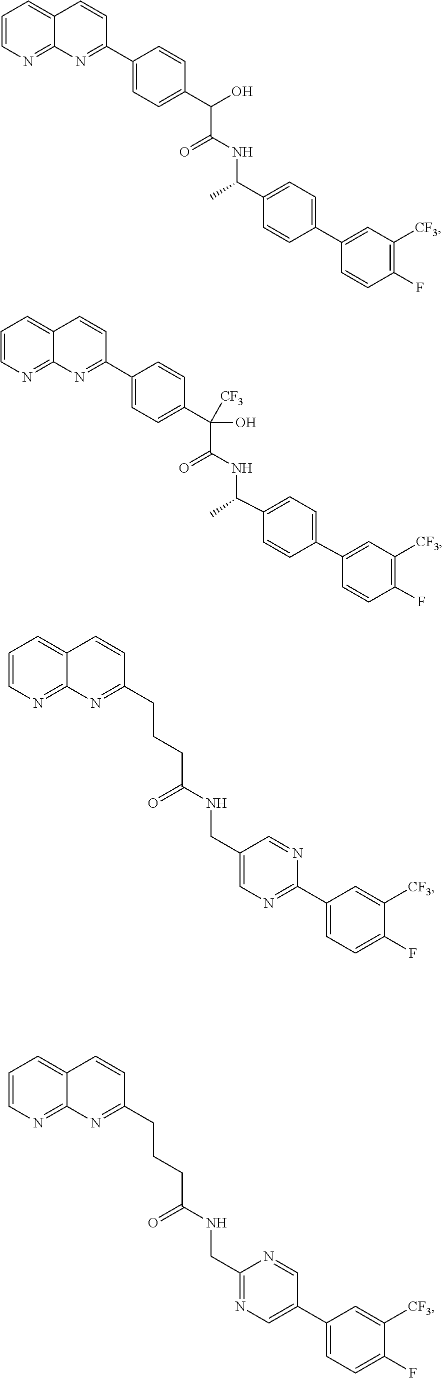 [SMILES: C[C@H](NC(=O)C(O)c1ccc(-c2ccc3cccnc3n2)cc1)c1ccc(-c2ccc(F)c(C(F)(F)F)c2)cc1 ; C[C@H](NC(=O)C(O)(c1ccc(-c2ccc3cccnc3n2)cc1)C(F)(F)F)c1ccc(-c2ccc(F)c(C(F)(F)F)c2)cc1 ; O=C(CCCc1ccc2cccnc2n1)NCc1cnc(-c2ccc(F)c(C(F)(F)F)c2)nc1 ; O=C(CCCc1ccc2cccnc2n1)NCc1ncc(-c2ccc(F)c(C(F)(F)F)c2)cn1] 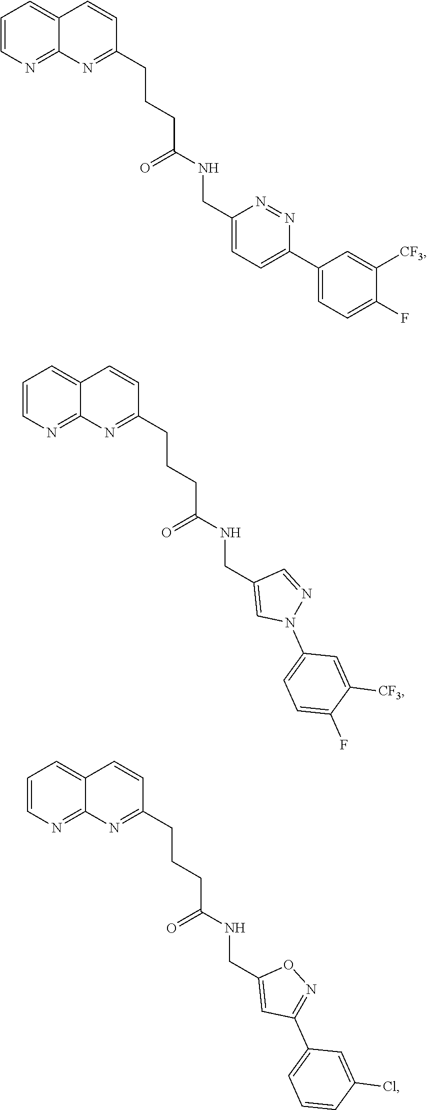 [SMILES: O=C(CCCc1ccc2cccnc2n1)NCc1ccc(-c2ccc(F)c(C(F)(F)F)c2)nn1 ; O=C(CCCc1ccc2cccnc2n1)NCc1cnn(-c2ccc(F)c(C(F)(F)F)c2)c1 ; O=C(CCCc1ccc2cccnc2n1)NCc1cc(-c2cccc(Cl)c2)no1] 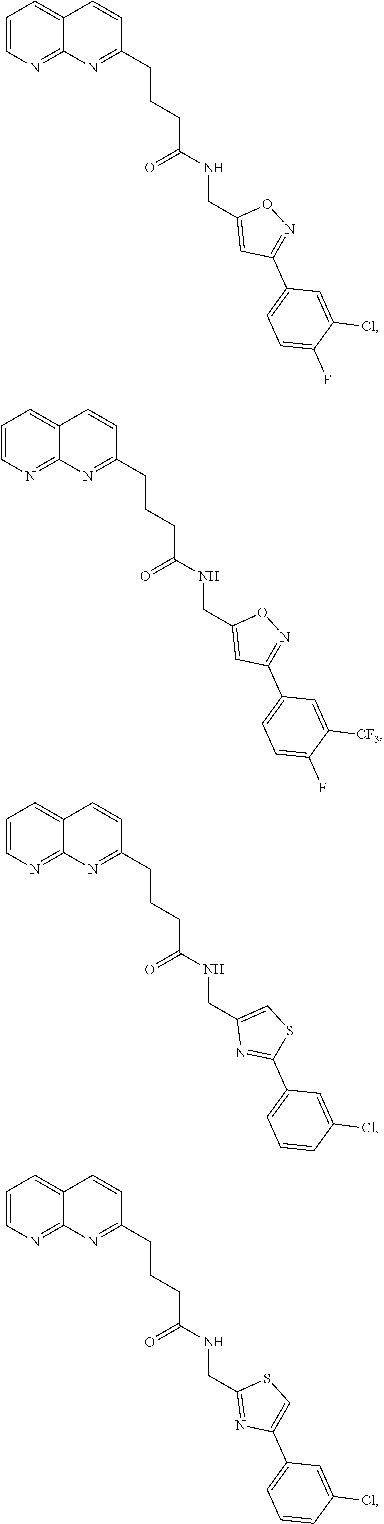 [SMILES: O=C(CCCc1ccc2cccnc2n1)NCc1cc(-c2ccc(F)c(Cl)c2)no1 ; O=C(CCCc1ccc2cccnc2n1)NCc1cc(-c2ccc(F)c(C(F)(F)F)c2)no1 ; O=C(CCCc1ccc2cccnc2n1)NCc1csc(-c2cccc(Cl)c2)n1 ; O=C(CCCc1ccc2cccnc2n1)NCc1nc(-c2cccc(Cl)c2)cs1] 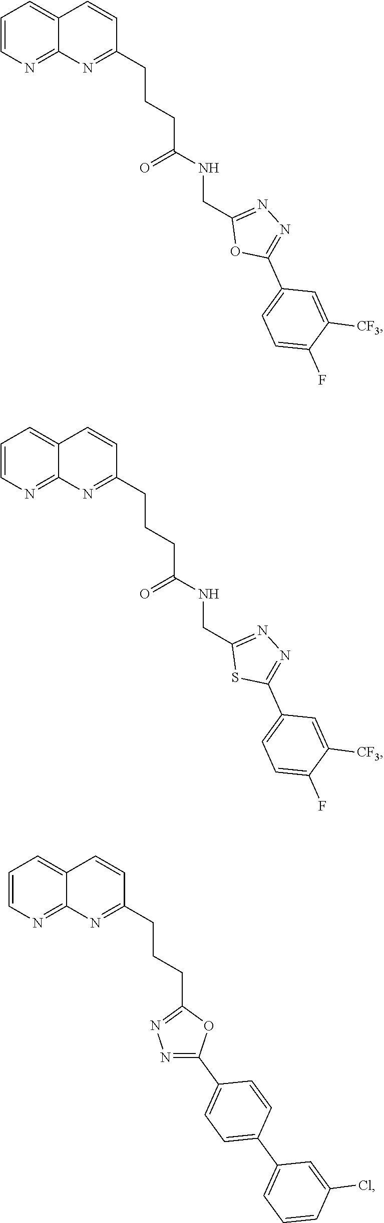 [SMILES: O=C(CCCc1ccc2cccnc2n1)NCc1nnc(-c2ccc(F)c(C(F)(F)F)c2)o1 ; O=C(CCCc1ccc2cccnc2n1)NCc1nnc(-c2ccc(F)c(C(F)(F)F)c2)s1 ; Clc1cccc(-c2ccc(-c3nnc(CCCc4ccc5cccnc5n4)o3)cc2)c1] 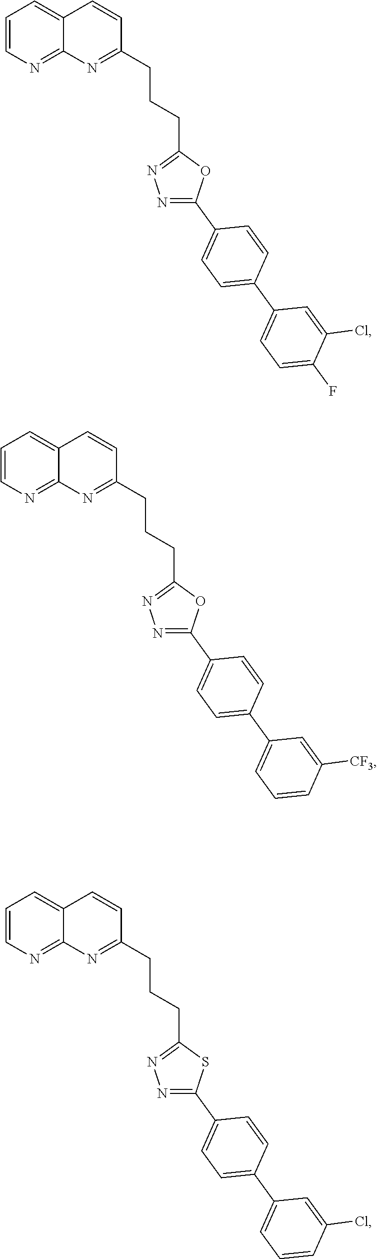 [SMILES: Fc1ccc(-c2ccc(-c3nnc(CCCc4ccc5cccnc5n4)o3)cc2)cc1Cl ; FC(F)(F)c1cccc(-c2ccc(-c3nnc(CCCc4ccc5cccnc5n4)o3)cc2)c1 ; Clc1cccc(-c2ccc(-c3nnc(CCCc4ccc5cccnc5n4)s3)cc2)c1] 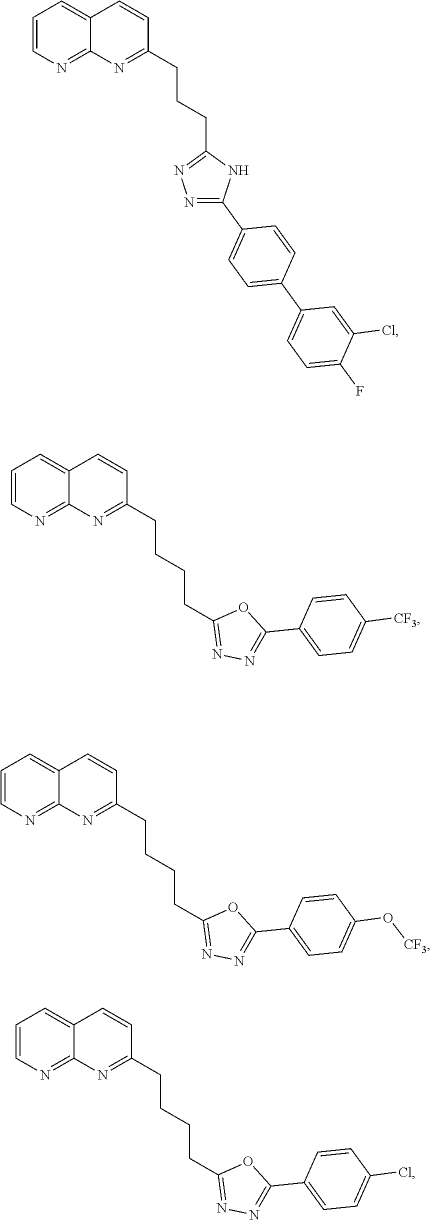 [SMILES: Fc1ccc(-c2ccc(-c3nnc(CCCc4ccc5cccnc5n4)[nH]3)cc2)cc1Cl ; FC(F)(F)c1ccc(-c2nnc(CCCCc3ccc4cccnc4n3)o2)cc1 ; FC(F)(F)Oc1ccc(-c2nnc(CCCCc3ccc4cccnc4n3)o2)cc1 ; Clc1ccc(-c2nnc(CCCCc3ccc4cccnc4n3)o2)cc1] 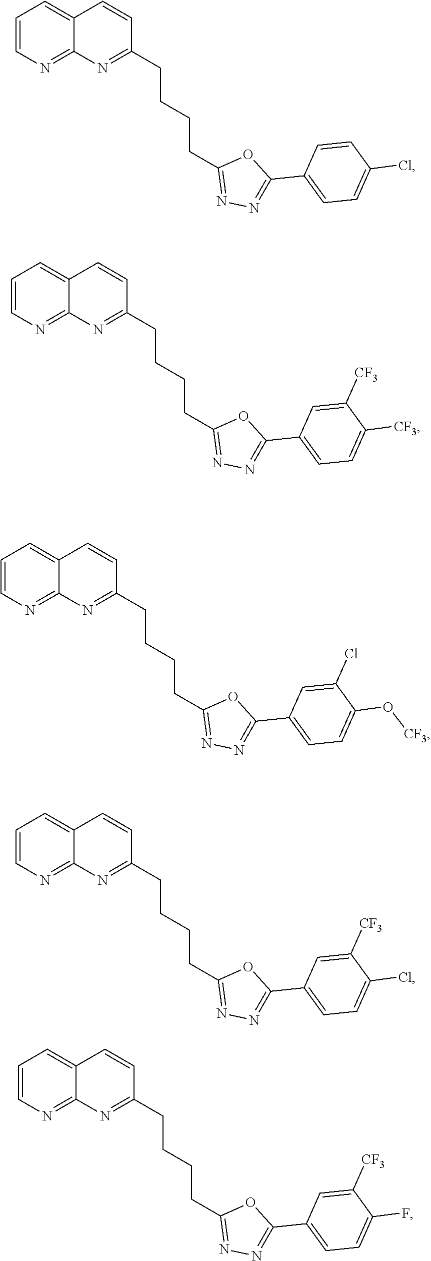 [SMILES: Clc1ccc(-c2nnc(CCCCc3ccc4cccnc4n3)o2)cc1 ; FC(F)(F)c1ccc(-c2nnc(CCCCc3ccc4cccnc4n3)o2)cc1C(F)(F)F ; FC(F)(F)Oc1ccc(-c2nnc(CCCCc3ccc4cccnc4n3)o2)cc1Cl ; FC(F)(F)c1cc(-c2nnc(CCCCc3ccc4cccnc4n3)o2)ccc1Cl ; Fc1ccc(-c2nnc(CCCCc3ccc4cccnc4n3)o2)cc1C(F)(F)F] 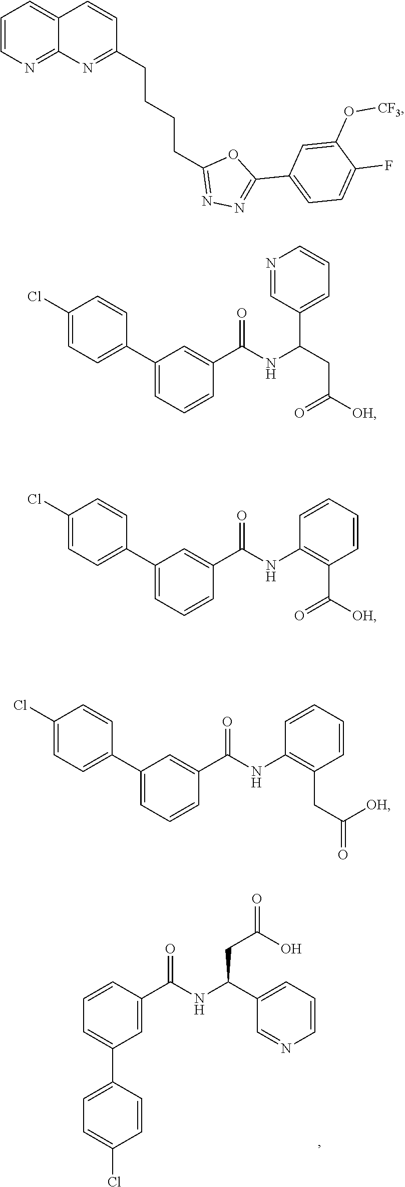 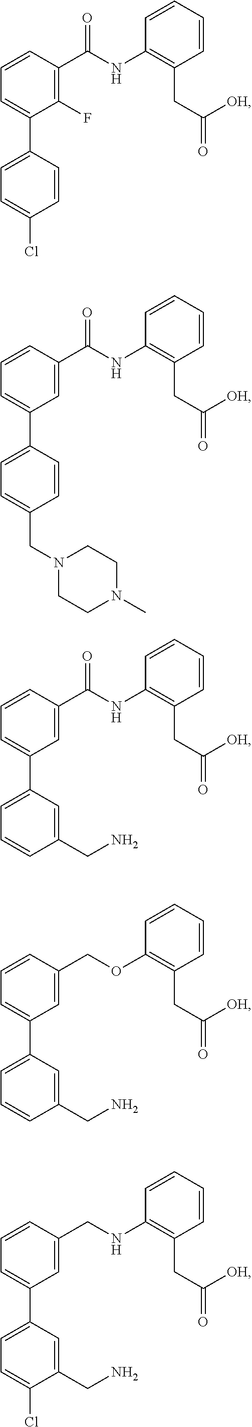 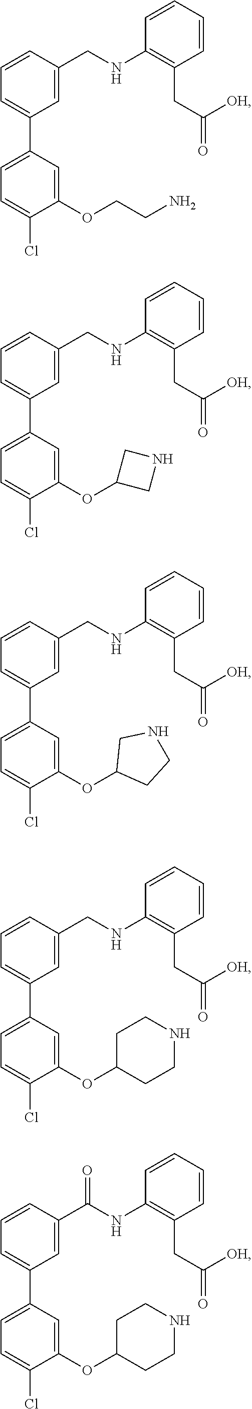 [SMILES: NCCOc1cc(-c2cccc(CNc3ccccc3CC(=O)O)c2)ccc1Cl ; O=C(O)Cc1ccccc1NCc1cccc(-c2ccc(Cl)c(OC3CNC3)c2)c1 ; O=C(O)Cc1ccccc1NCc1cccc(-c2ccc(Cl)c(OC3CCNC3)c2)c1 ; O=C(O)Cc1ccccc1NCc1cccc(-c2ccc(Cl)c(OC3CCNCC3)c2)c1 ; O=C(O)Cc1ccccc1NC(=O)c1cccc(-c2ccc(Cl)c(OC3CCNCC3)c2)c1] 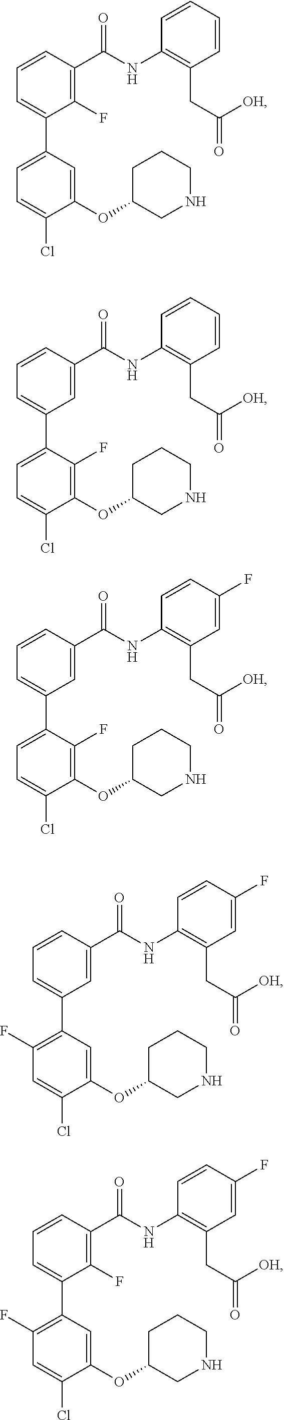 [SMILES: O=C(O)Cc1ccccc1NC(=O)c1cccc(-c2ccc(Cl)c(O[C@@H]3CCCNC3)c2)c1F ; O=C(O)Cc1ccccc1NC(=O)c1cccc(-c2ccc(Cl)c(O[C@@H]3CCCNC3)c2F)c1 ; O=C(O)Cc1cc(F)ccc1NC(=O)c1cccc(-c2ccc(Cl)c(O[C@@H]3CCCNC3)c2F)c1 ; O=C(O)Cc1cc(F)ccc1NC(=O)c1cccc(-c2cc(O[C@@H]3CCCNC3)c(Cl)cc2F)c1 ; O=C(O)Cc1cc(F)ccc1NC(=O)c1cccc(-c2cc(O[C@@H]3CCCNC3)c(Cl)cc2F)c1F]
and combinations thereof.

6. The method of claim 1, wherein the succinate/succinate receptor 1 inhibitor is selected from the group consisting of 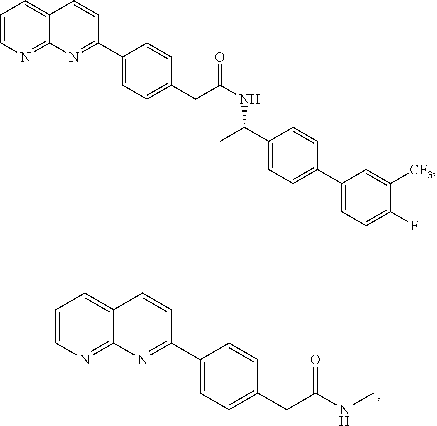 [SMILES: C[C@H](NC(=O)Cc1ccc(-c2ccc3cccnc3n2)cc1)c1ccc(-c2ccc(F)c(C(F)(F)F)c2)cc1 ; CNC(=O)Cc1ccc(-c2ccc3cccnc3n2)cc1]
and succinate salts thereof.

7. The method of claim 6, wherein the succinate/succinate receptor 1 inhibitor is

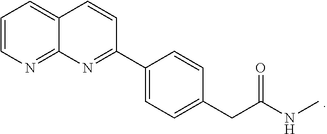 [SMILES: CNC(=O)Cc1ccc(-c2ccc3cccnc3n2)cc1]

8. The method of claim 7, wherein 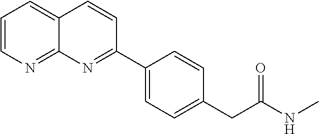 [SMILES: CNC(=O)Cc1ccc(-c2ccc3cccnc3n2)cc1]
is administered at a concentration of 1-10 mg/mL.

9. The method of claim 1, wherein the composition is a film, a gel, a capsule, a tablet, a plurality of nanoparticles, or liquid composition.

10. The method of claim 1, wherein the composition is topically applied in the oral cavity of the individual.

11. A method for reducing the expression of pro-inflammatory cytokines, comprising administering to the individual a composition comprising a succinate/succinate receptor 1 inhibitor,
wherein expression of the pro-inflammatory cytokines is reduced or inhibited.

12. The method of claim 11, wherein the composition is administered to a location other than the brain of the individual.

13. The method of claim 11, wherein the one or more pro-inflammatory cytokines are TNFα, IL1β, or a combination thereof.

14. A composition comprising a succinate/succinate receptor 1 inhibitor and one or more polymers, wherein the inhibitor of succinate/succinate receptor has the following structure or a succinate salt thereof: 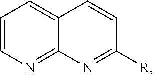
wherein R is selected from the group consisting of a hydrogen atom, an alkyl group, COOR′ group, and 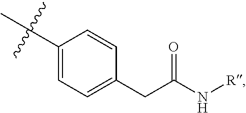
wherein R′ is an alkyl group and R″ is selected from the group consisting of a hydrogen atom, an alkyl group, and 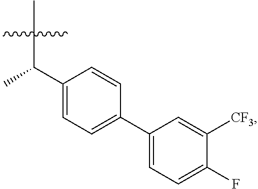 [SMILES: *[C@@H](C)c1ccc(-c2ccc(F)c(C(F)(F)F)c2)cc1]
with the proviso the composition is not a thin film having a thickness of from 0.05 mm to 0.4 mm.

15. The composition of claim 14, wherein the succinate/succinate receptor 1 inhibitor is selected from the group consisting of 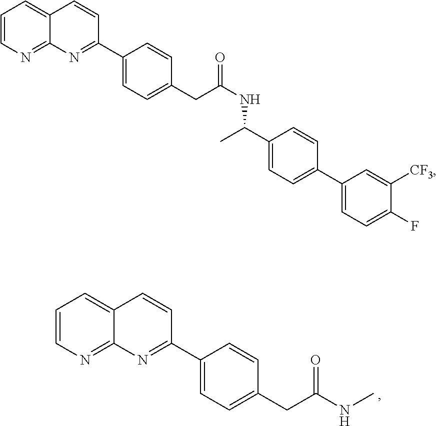 [SMILES: C[C@H](NC(=O)Cc1ccc(-c2ccc3cccnc3n2)cc1)c1ccc(-c2ccc(F)c(C(F)(F)F)c2)cc1 ; CNC(=O)Cc1ccc(-c2ccc3cccnc3n2)cc1]
and succinate salts thereof.

16. The composition of claim 15, wherein the succinate/succinate receptor 1 inhibitor is

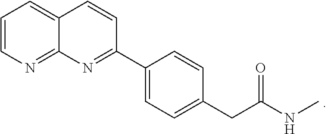 [SMILES: CNC(=O)Cc1ccc(-c2ccc3cccnc3n2)cc1]

17. The composition of claim 14, wherein the one or more polymers are chosen from poly lactic glycolic acid (PLGA), polycaprolactone (PCL), poly-ε-caprolactone ester terminated, poly(D,L-lactide-co-glycolide, poly(D,L-lactide-co-glycolide ester terminated, poly(D,L-lactide, poly(D,L-lactide) ester terminated, chitosan, starch, polylactic acid, alginate, and combinations thereof.

18. The composition of claim 14, further comprising a solvent, wherein the succinate/succinate receptor 1 inhibitor is 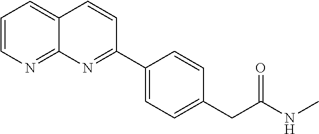 [SMILES: CNC(=O)Cc1ccc(-c2ccc3cccnc3n2)cc1]
the polymer is PLGA, and the composition further comprises a solvent, wherein the solvent is 30% solution (by volume) of benzyl benzoate in N-methyl-2-pyrrolidinone.

19. The composition of claim 18, wherein the succinate/succinate receptor 1 inhibitor is 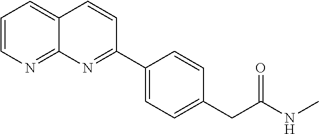 [SMILES: CNC(=O)Cc1ccc(-c2ccc3cccnc3n2)cc1]
having a concentration of 5.0 mg/mL,
the polymer is PLGA having a concentration of 10% w/v, and the solvent is a 30% solution (by volume) of benzyl benzoate in N-methyl-2-pyrrolidinone.
